(19) United States
(12) Patent Application Publication
(10) Pub. No.: US 2026/0218128 A1
(43) Pub. Date: Jul. 30, 2026

(54) NKG2D ENGINEERED CELL AND COMPOSITION THEREOF

(71) Applicant: CARSGEN LIFE SCIENCES CO., LTD., Shanghai (CN)

(72) Inventors: Zonghai LI, Shanghai (CN); Hua JIANG, Shanghai (CN); Yi WANG, Shanghai (CN); Yu ZHANG, Shanghai (CN); Liang ZHOU, Shanghai (CN)

(73) Assignee: CARSGEN LIFE SCIENCES CO., LTD., Shanghai (CN)

(21) Appl. No.: 19/144,198

(22) PCT Filed: Dec. 27, 2023
(86) PCT No.: PCT/CN2023/142272

§ 371 Date: Jun. 27, 2025

(30) Foreign Application Priority Data
Dec. 27, 2022 (CN) 202211692424.X
Jan. 29, 2023 (CN) 202310089423.4
Mar. 9, 2023 (CN) 202310225670.2

(51) Int. Cl. C12N 5/0783; A61K 40/11; A61K 40/31; A61K 40/42; A61P 35/00; C07K 14/705; C07K 16/28; C07K 16/30

(52) U.S. Cl. CPC C12N 5/0636; A61K 40/11; A61K 40/31; A61K 40/4202; A61P 35/00; C07K 14/705; C07K 16/28; C07K 16/2803; C07K 16/2827; C07K 16/2851; C07K 16/2866; C07K 16/303; C07K 2317/622; C07K 2319/03; C12N 2510/00

(57) ABSTRACT

An engineered immune cell for use in immunotherapy and a preparation method therefor. The engineered immune cell is more persistent in a host organism and has a high transplantation survival rate.

### TECHNICAL FIELD

The present application relates to a cell with anti-transplant rejection function, also relates to a method for resisting transplant immune rejection, and particularly to a method for resisting NK cell immune rejection.

### CROSS-REFERENCES

This application claims the priority to Chinese patent application No. CN202211692424.X filed on Dec. 27, 2022, Chinese patent application No. CN202310089423.4 filed on Jan. 29, 2023 and Chinese patent application No. CN202310225670.2 filed on Mar. 9, 2023, the contents of which are incorporated herein in their entirety.

### SEQUENCE LISTING FILE SUBMITTED SIMULTANEOUSLY

The entire contents of the following XML file are incorporated herein by reference in their entirety: Sequence Listing in Computer Readable Format (CRF) (Name: FG00927PCT-Sequence Listing.xml, Date: 20231227, Size: 157 KB).

### BACKGROUND ART

Due to the immunogenetic differences between the donor and the recipient, during exogenous donor transplantation, as an exogenous graft, the donor may also be recognized and attacked by the immune cells in the recipient, thereby inhibiting or eliminating the exogenous graft and generating a host-versus-graft reaction (HVGR). By knocking out the MHC molecules in the cells of the graft, rejection reactions of host T cells to the graft can be effectively resisted, but it may cause rejection reactions from other immune cells in the host. For example, in allogeneic cell transplantation, the lack of MHC-I molecules in allogeneic cells will lead to NK cell rejection in the host, thereby enhancing the clearance of allogeneic cells. Therefore, how to effectively prevent the immune rejection reaction of host NK cells is crucial to the development of allogeneic cell transplantation therapy.

### SUMMARY OF THE INVENTION

After extensive and in-depth research, the inventors unexpectedly discovered that expressing a chimeric receptor targeting the NKG2D ligand on immune cells can resist host NK cell attacks, prolong the survival time of the immune cells in and outside the host body, and enhance the anti-tumor effect. This application was completed on this basis.

The first aspect of the present application provides a chimeric polypeptide, comprising:
a) a binding domain capable of specifically binding to a target molecule;
b) a receptor regulatory domain comprising a cleavage site,
the receptor regulatory domain comprises an extracellular domain and a transmembrane domain, wherein the extracellular domain comprises one or more target molecule binding-induced cleavage sites B, and the transmembrane domain comprises one or more target molecule binding-induced cleavage sites A; and
c) an intracellular domain,
wherein, the binding of the binding domain to the target molecule can induce the cleavage of the cleavage site A and/or B of the receptor regulatory domain, thereby releasing the intracellular domain; the extracellular domain of the receptor regulatory domain does not derived from the Notch protein, and a non-target molecule binding-induced cleavage of the chimeric polypeptide is reduced by mutating the extracellular domain of the receptor regulatory domain or changing steric hindrance of the extracellular domain.

In one embodiment, by mutating the extracellular domain of the receptor regulatory domain or changing the steric hindrance of the extracellular domain, the non-target molecule binding-induced cleavage of the chimeric polypeptide is reduced, while the cleavage of the target molecule binding-induced cleavage site B is increased, thereby increasing the cleavage of the cleavage site A and releasing the intracellular domain.

In one embodiment, by mutating the extracellular domain of the receptor regulatory domain or changing the steric hindrance of the extracellular domain, the non-target molecule binding-induced cleavage of the chimeric polypeptide is reduced, while the cleavage of the target molecule binding-induced cleavage site A is increased to release the intracellular domain.

In one embodiment, the mutation is located at a non-target molecule binding-induced cleavage site in the extracellular domain.

In one embodiment, the mutation comprises a deletion, an insertion and/or a point mutation.

In one embodiment, the steric hindrance of the extracellular domain is altered by adding EGF repeat sequences to the extracellular domain to reduce non-target molecule binding-induced cleavage.

In one embodiment, the extracellular domain comprises an extracellular domain derived from Jagged2, EphrinB2, APLP1, APLP2, APP, CD44, CSF1R, CXCL16, CX3CL1, Delta1, E-cadherin, EphB2, EphrinB1, Growth hormone receptor, HLA-A2, IFNaR2, IL1R2, L1, LRP, LRP2, LRP6, N-cadherin, Nectin1α, NRADD, p75-NTR, Pcdh α4, Pcdh γ-C3, PTPκ, PTP-LAR, SorCS1b, SorLA, Sortilin, ApoER2, PKHD1, ErbB4, IFNaR2, VEGF-R1, or VLDLR, or any combination thereof, or a fragment of the extracellular domain of any of the above proteins or any combination thereof, or a variant of the extracellular domain of any of the above proteins or any combination thereof, or a truncated structure of the extracellular domain of any of the above proteins or any combination thereof.

In one embodiment, the extracellular domain comprises an amino acid sequence having at least 80%, 85%, 90%, 95%, 99%, 100% sequence homology with any one of SEQ ID NO: 1, 3, 4, 5, 6, 7, 8, 9, 10 or 65.

The second aspect of the present application provides a chimeric polypeptide, comprising:
a) a binding domain capable of specifically binding to a target molecule;
b) a receptor regulatory domain comprising one or more cleavage sites,
the receptor regulatory domain comprises an extracellular domain and a transmembrane domain, wherein the extracellular domain comprises an amino acid sequence having at least 80%, 85%, 90%, 95%, 99%, 100% identity with any one of SEQ ID NO: 1, 3, 4, 5, 6, 7, 8, 9, 10 or 65, and the transmembrane domain comprises one or more target molecule binding-induced cleavage sites A; and
c) an intracellular domain,
wherein the binding of the binding domain to the target molecule can induce the cleavage of the receptor regulatory domain, thereby releasing the intracellular domain.

In one embodiment, the transmembrane domain further comprises a stop transfer sequence (STS).

In one embodiment, the transmembrane domain of the receptor regulatory domain comprises a γ-secretase cleavage site.

In one embodiment, the transmembrane domain of the receptor regulatory domain comprises a Notch transmembrane domain.

In one embodiment, the STS comprises an amino acid sequence having at least 80% sequence homology with any one of SEQ ID NO: 23, 24, 25, 26, or 27.

In one embodiment, the transmembrane domain of the receptor regulatory domain comprises an amino acid sequence having at least 80% sequence homology with any one of SEQ ID NO: 11, 12, 13, 14, 15, 16, 17, 18, 19, 20, 21, or 22.

In one embodiment, the binding domain comprises an antigen binding domain capable of binding to a target molecule on the surface of a target cell, or the binding domain comprises a ligand portion capable of binding to a receptor.

In one embodiment, the target molecule is selected from the group consisting of: differentiation marker clusters, cell surface receptors, adhesion proteins, integrins, mucins, lectins, tumor antigens, and tissue-specific antigens.

In one embodiment, the target molecule is a tumor antigen or a tissue-specific antigen.

In one embodiment, the binding domain is selected from: an antibody, a receptor, a ligand of a receptor, a cell adhesion molecule, a non-antibody molecular scaffold, or any combination thereof.

In one embodiment, the antibody is a single domain antibody, a single chain antibody, a double chain antibody, a triple chain antibody, a mini antibody, a F(ab′)₂ fragment, a F (ab) v fragment, a scFv, a single domain antibody (sdAb) and a functional fragment thereof, or any combination thereof.

In one embodiment, the binding domain specifically binds to a tumor antigen selected from the group consisting of: Mesothelin, FAP, Claudin18.2, CLL1, CD19, GPC3, WT1, EGFR, BCMA, CD7, NKG2D-Ligand, CD19, B7H3, ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRVIII, ELF2M, EpCAM, EphA2, FLT3, GD2, GD3, GM3, GPRC5D, HER2 (ERBB2), IGLL1, IL 11Ra, IL13Ra2, CD 117, MUC1, NCAM, PAP, PDGFR-b, PRSS21, PSCA, PSMA, ROR1, SIRPa, SSEA-4, TAG72, TEM1/CD248, TEM7R, TSHR, VEGFR2, ALPI, cMet and Axl.

In one embodiment, the binding domain specifically binds to a tissue-specific marker selected from the group consisting of: a brain tissue marker MOG, a liver tissue marker ASGR1, and a prostate tissue marker PSA.

In one embodiment, the binding domain comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity or homology with any one of the amino acid sequences represented by SEQ ID NO: 43, 68, 69, 70, 71, 72, 73, 74, 75, 76, 77, 78, 79, 80, 81, 82 or 83.

In one embodiment, the intracellular domain comprises a transcription factor, a site-specific nuclease, a recombinase, an inhibitory immune receptor, an activating immune receptor, or any combination thereof.

In one embodiment, the transcription factor is selected from Gal4-VP16, Gal4-VP64, tetR-VP64, ZFHD1-VP64, Gal4-KRAB, HAP1-VP16, or any combination thereof.

In one embodiment, additional proteolytic cleavage sites, signal sequences, detectable tags, tumor-specific cleavage sites, disease-specific cleavage sites, and combinations thereof are also comprised.

In one embodiment, the receptor regulatory domain comprises an amino acid sequence having at least 80%, 85%, 90%, or 95% homology with any one of the amino acid sequences shown after sequentially connecting the extracellular domain represented by SEQ ID NO: 1, 2, 3, 4, 5, 6, 7, 8, 9, 10, or 65, and the transmembrane domain represented by SEQ ID NO: 11, 12, 13, 14, 15, 16, 17, 18, 19, 20, 21, or 22.

In one embodiment, the chimeric polypeptide comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or homology with any one of the amino acid sequences shown after sequentially connecting the extracellular domain represented by SEQ ID No: 1, 2, 3, 4, 5, 6, 7, 8, 9, 10 or 65, the transmembrane domain represented by SEQ ID NO: 11, 12, 13, 14, 15, 16, 17, 18, 19, 20, 21 or 22, and the intracellular domain represented by SEQ ID NO: 28.

In one embodiment, the chimeric polypeptide comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or homology with any one of the amino acid sequences represented by SEQ ID NO:44, 45, 46, 47, 48 or 49.

In one embodiment, the target cell is a pathogen.

In one embodiment, the target cell is a human cell.

In one embodiment, the human cell is a tumor cell or a normal tissue cell.

The third aspect of the present application provides a nucleic acid molecule comprising a nucleotide sequence encoding the chimeric polypeptide of the first, second, or eighteenth aspect, the antibody of the sixteenth aspect, or the immunoconjugate of the seventeenth aspect.

In one embodiment, the nucleic acid molecule is constructed in an expression cassette or an expression vector.

In one embodiment, the expression vector comprises a viral vector or a transposon vector.

In one embodiment, the viral vector is a lentiviral vector, an adenoviral vector, an adeno-associated viral vector, or a retroviral vector.

The fourth aspect of the present application provides an engineered cell comprising the chimeric polypeptides of the first, second or eighteenth aspect, the antibody of the sixteenth aspect, the immunoconjugate of the seventeenth aspect, and/or the nucleic acid molecule of the third aspect.

In one embodiment, the engineered cell is an immune cell, a neuron, an epithelial cell, an endothelial cell, or a stem cell.

In one embodiment, the engineered cells is a B cell, a monocyte, a natural killer cell, a basophil, an eosinophil, a neutrophil, a dendritic cell, a macrophage, a T cell, or any combination thereof; preferably, the engineered cell is an autologous or allogeneic cell.

In one embodiment, it further comprises an expression cassette encoding an exogenous gene operatively linked to the intracellular domain of the chimeric polypeptide, wherein the intracellular domain of the chimeric polypeptide regulates the expression of the exogenous gene.

In one embodiment, the exogenous gene is expressed under the control of a promoter regulated by GAL-4, tetR, ZFHD1, HNF1A or HAP1.

In one embodiment, the exogenous gene expression product is selected from: non-coding RNA, cytokine, cytotoxin, chemokine, immunomodulator, pro-apoptotic factor, anti-apoptotic factor, hormone, differentiation factor, dedifferentiation factor, recombinant TCR, CAR, reporter gene, or any combination thereof.

In one embodiment, the binding domain of the chimeric polypeptide specifically binds to a first target molecule, the exogenous gene expression product is CAR, and the CAR specifically binds to a second target molecule different from the first target molecule, wherein the first target molecule and the second target molecule can be respectively selected from the group consisting of: Mesothelin, FAP, Claudin18.2, CLL1, CD19, GPC3, WT1, EGFR, BCMA, CD7, NKG2D-Ligand, MOG, CD19, B7H3, ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRVIII, ELF2M, EpCAM, EphA2, Ephrin B2, FAP, FLT3, GD2, GD3, GM3, GPRC5D, HER2 (ERBB2), IGLL1, IL 11Ra, IL13Ra2, CD 117, MUC1, NCAM, PAP, PDGFR-b, PRSS21, PSCA, PSMA, ROR1, SIRPa, SSEA-4, TAG72, TEM1/CD248, TEM7R, TSHR, VEGFR2, ALPI, cMet and Axl.

In one embodiment, the first target molecule and the second target molecule are selected from any of the following combinations: ASGR1 and GPC3, EGFRVIII and IL 13Ra2, EGFRVIII and B7H3, Mesothelin and Claudin18.2, Claudin18.2 and Mesothelin, FAP and Claudin18.2, CLL1 and NKG2D, CD123 and NKG2D, CLL1 and NKG2D ligand, CD123 and NKG2D ligand, and MOG and B7H3.

In one embodiment, the chimeric polypeptide and CAR respectively comprise the sequences represented by SEQ ID NOs: 46 and 50; or the sequences represented by SEQ ID NOs: 47 and 50; or the sequences represented by SEQ ID NOs: 48 and 51; or the sequences represented by SEQ ID NOs: 48 and 52; or the sequences represented by SEQ ID NOs: 48 and 53; or the sequences represented by SEQ ID NOs: 48 and 54; or the sequences represented by SEQ ID NOs: 49 and 51; or the sequences represented by SEQ ID NOs: 49 and 52; or the sequences represented by SEQ ID NOs: 49 and 53; or the sequences represented by SEQ ID NOs: 49 and 54.

In one embodiment, the cytokine is IL-2, IL-7, IL-9, IL-12, IL-15, IL-18, CCL21, or any combination thereof.

In one embodiment, the engineered cell comprises a nucleic acid sequence having at least 80% sequence homology with any one of SEQ ID NO: 55, 56, 57, 58 or 59 or an amino acid sequence translated therefrom.

In one embodiment, the engineered cell also expresses an exogenous receptor targeting NK cells.

In one embodiment, the exogenous receptor targets a NK cell marker selected from: NKG2 receptor family, such as NKG2A, NKG2D, NKG2C, etc.; killer immunoglobulin-like receptor (KIR) family, such as KIR2DL1, KIR2DL2/3, KIR2DL4, KIR2DL5, KIR3DL1, 15KIR3DL2, KIR2DS1, KIR2DS2/S3, KIR2DS4, KIR2DS5, KIR3DS1, etc.; natural cytotoxicity receptors (NCR), such as NKP30, NKP44, NKP46, NKp80, etc.; and other NK cell-specifically expressed antigens, such as CD38, CD159a, CD159c, CD94, CD158, CD56, LIR/ILT2, CD244, CD226, CD2, CD16, CD161, TIGIT, CS1, and NKG2D ligand.

In one embodiment, the exogenous receptor comprises a CAR or a recombinant TCR.

In one embodiment, the exogenous receptor comprises NKG2A-CAR, CD94-CAR, CS1-CAR, TIGIT-CAR, NKG2D-CAR that recognizes NKG2D ligand, or any combination thereof.

The fifth aspect of the present application provides a composition comprising the engineered cell of the fourth aspect, wherein the composition further comprises another engineered cell targeting a NK cell marker.

In one embodiment, the other engineered cell targets a NK cell marker selected from: NKG2 receptor family, such as NKG2A, NKG2D, NKG2C, etc.; killer immunoglobulin-like receptor (KIR) family, such as KIR2DL1, KIR2DL2/3, KIR2DL4, KIR2DL5, KIR3DL1, 15KIR3DL2, KIR2DS1, KIR2DS2/S3, KIR2DS4, KIR2DS5, KIR3DS1, etc.; natural cytotoxicity receptors (NCR), such as NKP30, NKP44, NKP46, NKp80, etc.; and other NK cell-specifically expressed antigens, such as CD38, CD159a, CD159c, CD94, CD158, CD56, LIR/ILT2, CD244, CD226, CD2, CD16, CD161, TIGIT, CS1, and NKG2D ligand.

In one embodiment, the other cell expresses NKG2A-CAR, CD94-CAR, CS1-CAR, TIGIT-CAR, NKG2D-CAR that recognizes a NKG2D ligand, or any combination thereof.

In one embodiment, the engineered cell or another engineered cell is independently selected from: an immune cell, a neuron, an epithelial cell, an endothelial cell or a stem cell.

In one embodiment, the immune cell is selected from: a B cell, a monocyte, a natural killer cell, a basophil, an eosinophil, a neutrophil, a dendritic cell, a macrophage, a T cell, or any combination thereof.

In one embodiment, the T cell is selected from: an allogeneic T cell or an autologous T cell.

In one embodiment, the engineered cell and/or another engineered cell is an engineered cell and/or another engineered cell comprising any one, two, three or four of the following a)-d):
a) low expression or no expression of endogenous HLA-I molecules;
b) low expression or no expression of endogenous TCR molecules;
c) low expression or no expression of endogenous HLA-II molecules; and/or
d) low expression or no expression of endogenous NKG2A molecules.

In one embodiment, the engineered cell and/or another engineered cell is an engineered cell and/or another engineered cell comprising any one, two, three or four of the following a) to d):
a) low expression or no expression of endogenous HLA-I molecules, including knockout of genes encoding HLA-I proteins;
b) low expression or no expression of endogenous TCR molecules, including knockout of genes encoding TCR proteins;
c) low expression or no expression of endogenous HLA-II molecules, including knockout of genes encoding HLA-II proteins; and/or
d) low expression or no expression of endogenous NKG2A molecules, including knockout of the gene encoding NKG2A protein.

In one embodiment, the engineered cell and/or another engineered cell is an engineered cell and/or another engineered cell comprising any one of a) to f):
a) knockout of endogenous B2M using CRISPR technology;
b) knockout of endogenous B2M/TCR using CRISPR technology;
c) knockout of endogenous B2M/TCR/CIITA using CRISPR technology;
d) knockout of endogenous B2M/TCR/NKG2A using CRISPR technology;
e) knockout of endogenous B2M/TCR/FAS using CRISPR technology; or
f) knockout of endogenous B2M/TCR/CIITA/NKG2A using CRISPR technology.

The sixth aspect of the present application provides a pharmaceutical composition, comprising: a pharmaceutically acceptable carrier; and further comprising the nucleic acid molecule of the third aspect and/or the engineered cell of the fourth aspect and/or the composition of the fifth aspect and/or the biological material of the nineteenth aspect.

The seventh aspect of the present application provides a method for regulating the activity of an engineered cell, comprising:
a) providing the engineered cell according to the fourth aspect; and
b) contacting the engineered cell with a target molecule, wherein the binding of the target molecule to the binding domain of the chimeric polypeptide on the engineered cell induces cleavage of the proteolytic cleavage site of the chimeric polypeptide and releases the transcription factor in the intracellular domain of the chimeric polypeptide, thereby regulating the activity of the engineered cell.

In one embodiment, the activity of the engineered cell is any one or more selected from the group consisting of: cell proliferation, cell apoptosis, non-apoptotic cell death, cell differentiation, cell dedifferentiation, cell migration, cell adhesion and cell lytic activity.

In one embodiment, the transcription factor regulates expression of exogenous gene, the exogenous gene is selected from the group consisting of: genes of a chemokine, a chemokine receptor, a chimeric antigen receptor, a cytokine, a cytokine receptor, a differentiation factor, a growth factor, a growth factor receptor, a hormone, a metabolic enzyme, a pathogen-derived protein, a proliferation inducer, a receptor, a RNA-guided nuclease, a site-specific nuclease, a T cell receptor, a toxin, a toxin-derived protein, a transcription regulator, a transcription activator, a transcription repressor, a translation regulator, a translation activator, a translation repressor, an activating immune receptor, an antibody, an apoptosis inhibitor, an apoptosis inducer, a modified T cell receptor, an immune activator, an immunosuppressant, and an inhibitory immune receptor.

In one embodiment, the released transcription factor regulates the differentiation of the engineered cell, which is an immune cell, a stem cell, a progenitor cell, or a precursor cell.

In one embodiment, it is a method for treating a tumor.

The eighth aspect of the present application provides a method for activating an engineered cell, the method comprising:
contacting the engineered cell of the fourth aspect with a target molecule, wherein the binding domain of the chimeric polypeptide comprises an antibody that specifically binds to a first target molecule, and wherein the contact results in the release of the transcription factor activating protein in the intracellular domain of the chimeric polypeptide, which regulate the expression of CAR and/or recombinant TCR in the engineered cell, wherein the CAR and/or recombinant TCR specifically binds to a second target molecule different from the first target molecule and then activates the engineered cell.

In one embodiment, the first target molecule and the second target molecule are different tumor antigens; or the first target molecule is a molecule with tissue specificity and the second target molecule is a tumor antigen.

The ninth aspect of the present application provides a method for inhibiting the activity of a target cell in a subject, characterized in that it comprises: administering to the subject a therapeutically effective amount of the engineered cell of the fourth aspect, wherein the engineered cell inhibits the activity of the target cell in the subject.

In one embodiment, the target cell is a tumor cell.

In one embodiment, the target cell is an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T cell lymphoma cell, a renal cancer cell, a sarcoma cell, a gastric cancer cell, a hepatoma cell, a mesothelioma cell or a sarcoma cell.

In one embodiment, the target cell expresses low levels of a target molecule and the binding domain of the chimeric polypeptide specifically binds to the target molecule.

In one embodiment, the engineered cell further comprises a CAR, a recombinant TCR, an exogenous cytokine and/or a therapeutic monoclonal antibody that is triggered for transcriptional activation by the binding of the chimeric polypeptide to a target molecule.

In one embodiment, the target molecule comprises a first target molecule and a second target molecule, the chimeric polypeptide specifically binds to the first target molecule, and the CAR and/or recombinant TCR specifically binds to the second target molecule; the first target molecule and the second target molecule in the target cell express heterogeneous tumor antigens, or the first target molecule is a tissue-specific molecule and the second target molecule is a tumor antigen.

In one embodiment, the positive rate of the first target molecule in the target cell is lower than the positive rate of the second target molecule in the target cell.

In one embodiment, it can further improve the anti-tumor specificity of the engineered cell.

The tenth aspect of the present application provides a system or a medicine box or a kit for regulating cell activity, inhibiting a target cell or treating the health condition of a subject in need thereof, comprising: one or more of the following:
a) the chimeric polypeptide of any one of the first, second and eighteenth aspects;
b) the nucleic acid molecule of the third aspect;
c) the cell of the fourth aspect; and
d) the composition of the fifth aspect;
e) the pharmaceutical composition of the sixth aspect;
f) the antibody of the sixteenth aspect;
g) the immunoconjugate of the seventeenth aspect;
h) the biological material of the nineteenth aspect.

The eleventh aspect of the present application provides one or more of the following uses for treating a tumor:
a) the chimeric polypeptide of the first or second aspect;
b) the nucleic acid molecule of the third aspect;
c) the cell of the fourth aspect; and
d) the composition of the fifth aspect;
e) the pharmaceutical composition of the sixth aspect;
f) the antibody of the sixteenth aspect;
g) the immunoconjugate of the seventeenth aspect;
h) the biological material of the nineteenth aspect.

In one embodiment, the tumor is a solid tumor, a hematological tumor, a soft tissue tumor, or a metastatic lesion.

The twelfth aspect of the present application provides use of the chimeric polypeptide of the first or second aspect; the nucleic acid molecule of the third aspect; the cell of the fourth or thirteenth aspect; the composition of the fifth aspect; or the pharmaceutical composition of the sixth aspect, in the manufacture of a medicament for treating a health condition.

The thirteenth aspect of the present application provides an engineered cell that recognizes allogeneic immune cells, wherein the engineered cell expresses a molecule that recognizes an NKG2D ligand, and the expression of the molecule that recognizes an NKG2D ligand is regulatable.

In one embodiment, the molecule that recognizes a NKG2D ligand is a chimeric receptor.

In one embodiment, the chimeric receptor comprises the full-length NKG2D polypeptide or a fragment thereof, or the chimeric receptor comprises an antibody that recognizes a NKG2D ligand or a fragment thereof, preferably, the NKG2D fragment comprises the extracellular domain of NKG2D.

In one embodiment, the engineered cell is an immune cell, a neuron, an epithelial cell, an endothelial cell, or a stem cell.

In one embodiment, the engineered cell expresses a second chimeric receptor that does not recognize the NKG2D ligand, and after the second chimeric receptor recognizes the target molecule, it induces the expression of the molecule that recognizes the NKG2D ligand.

In one embodiment, the binding domain of the second chimeric receptor binds to the target molecule, inducing the cleavage of the second chimeric receptor, releasing transcription factors to regulate the expression of the molecule that recognizes the NKG2D ligand;
preferably, the second chimeric receptor comprises the chimeric polypeptide of the first aspect or the second aspect;
preferably, the second chimeric receptor comprises a synNotch polypeptide;
preferably, the second chimeric receptor comprises the sequence represented by SEQ ID NO: 44, 45, 46, 47, 48, 49, 60, 61, 62, 63, 64, 65, 66 or 67;
preferably, the transcription factor is selected from Gal4-VP16, Gal4-VP64, tetR-VP64, ZFHD1-VP64, Gal4-KRAB, HAP1-VP16, or any combination thereof.

In one embodiment, the target molecule comprises a tumor antigen and/or a pathogen antigen.

In one embodiment, the engineered cells are selected from the group consisting of autologous or allogeneic T cells, NK cells, NKT cells, macrophages, CIK cells, and stem cell-derived immune cells; preferably, the engineered cells are autologous or allogeneic T cells.

In one embodiment, the engineered cells further express a third chimeric receptor that recognizes a tumor antigen or a pathogen antigen.

In one embodiment, the allogeneic immune cells comprise NK cells and/or T cells.

In one embodiment, the engineered cell does not express HLA, or the HLA gene endogenously expressed in the cell is silenced; preferably, the HLA is an HLA-I class gene.

In one embodiment, the HLA-I gene is one or more selected from the group consisting of: HLA-A, HLA-B, HLA-C, and B2M; preferably, the HLA-I gene is B2M.

In one embodiment, the engineered cells are T cells with HLA-I gene and endogenous TCR gene silenced; preferably, the cells are B2M/TCR gene-silenced or B2M/TCR/FAS gene-silenced T cells; preferably, B2M and/or TRAC are knocked out using CRISPR/Cas9 technology, or B2M/TCR/FAS are knocked out using CRISPR/Cas9 technology.

In one embodiment, the chimeric receptor, the third chimeric receptor comprises a chimeric antigen receptor (CAR) and/or a recombinant T cell receptor (a recombinant TCR).

In one embodiment, the chimeric receptor, the third chimeric receptor comprises an extracellular domain, a transmembrane domain, and an intracellular signaling domain;
preferably, the engineered cells transmit signals through the intracellular signaling domain to mediate the killing of host immune cells, or to mediate resistance to the killing of host immune cells; preferably, the extracellular domain is bound to the transmembrane domain through a hinge.

In one embodiment, the chimeric receptor comprises:
(i) NKG2D polypeptide or a fragment thereof, the transmembrane domain of CD28 or CD8, the co-stimulatory signaling domain of CD28 and the intracellular domain of CD3ζ; and/or
(ii) NKG2D polypeptide or a fragment thereof, the transmembrane domain of CD28 or CD8, the co-stimulatory signaling domain of CD137 and the intracellular domain of CD3ζ; and/or
(iii) NKG2D polypeptide or a fragment thereof, the transmembrane domain of CD28 or CD8, the co-stimulatory signaling domain of CD28, the co-stimulatory signaling domain of CD137 and the intracellular domain of CD3ζ; and/or
(iv) NKG2D polypeptide or a fragment thereof, the transmembrane domain of CD28 or CD8 and the intracellular domain of CD3ζ.

In one embodiment, the engineered cell can enhance the survival and proliferation of a second T cell and/or a second CAR-T cell targeting tumor antigen introduced into a subject previously, simultaneously, or subsequently; as well as the killing of a tumor cell by the second T cell and/or the second CAR-T cell.

The fourteenth aspect of the present application provides an engineered cell expressing a molecule that recognizes a NKG2D ligand, which is used for preparing a medicament for eliminating allogeneic immune cells.

In one embodiment, the molecule that recognizes a NKG2D ligand is a chimeric receptor.

In one embodiment, the chimeric receptor comprises a full-length NKG2D polypeptide or a fragment thereof, or the chimeric receptor comprises an antibody that recognizes a NKG2D ligand or a fragment thereof.

In one embodiment, the engineered cell is an immune cell, a neuron, an epithelial cell, an endothelial cell, or a stem cell.

In one embodiment, the expression of the molecule that recognizes the NKG2D ligand is regulatable.

In one embodiment, the allogeneic immune cells comprise NK cells and/or T cells.

The fifteenth aspect of the present application provides a method for preventing or regulating transplant immune rejection, comprising administering the engineered cell of the thirteenth aspect, or administering the engineered cell having the use of the fourteenth aspect.

In one embodiment, the method is used to kill allogeneic immune cells; preferably, the allogeneic immune cells comprise NK cells and/or T cells.

The sixteenth aspect of the present application provides a fully human antibody that recognizes myelin oligodendrocyte glycoprotein (MOG), wherein the antibody is selected from the group consisting of:
(1) an antibody comprising a light chain variable domain, wherein the light chain variable domain comprises LCDR1 represented by SEQ ID NO: 84, and/or LCDR2 represented by SEQ ID NO: 85, and/or LCDR3 represented by any one of SEQ ID NO: 86, 87, 88, 89, 90, 91, 92, 93, 94, 95, 96, 97 or 98;
(2) an antibody comprising a heavy chain variable domain, wherein the heavy chain variable domain comprises the HCDR1 represented by SEQ ID NO: 99 or 100, and/or the HCDR2 represented by SEQ ID NO: 101 or 102, and/or the HCDR3 represented by any one of SEQ ID NO: 103, 104, 105, 106, 107, 108, 109, 110, 111, 112, 113, 114 or 115;
(3) an antibody comprising a light chain variable domain of the antibody described in (1) and a heavy chain variable domain of the antibody described in (2); and
(4) an antibody, which is a variant of the antibody of any one of (1) to (3), and has the same or similar activity as the antibody of any one of (1) to (3).

In one embodiment, the antibody is selected from:
(1) an antibody comprising LCDR1, LCDR2, LCDR3, HCDR1, HCDR2, and HCDR3 represented by SEQ ID NOs: 84, 85, 86, 99, 101, and 103, respectively; or
(2) an antibody comprising LCDR1, LCDR2, LCDR3, HCDR1, HCDR2, and HCDR3 represented by SEQ ID NOs: 84, 85, 87, 99, 101, and 104, respectively; or
(3) an antibody comprising LCDR1, LCDR2, LCDR3, HCDR1, HCDR2, and HCDR3 represented by SEQ ID NOs: 84, 85, 88, 99, 101, and 105, respectively; or
(4) an antibody comprising LCDR1, LCDR2, LCDR3, HCDR1, HCDR2, and HCDR3 represented by SEQ ID NOs: 84, 85, 89, 99, 101, and 106, respectively; or
(5) an antibody comprising LCDR1, LCDR2, LCDR3, HCDR1, HCDR2, and HCDR3 represented by SEQ ID NOs: 84, 85, 90, 99, 101, and 107, respectively; or
(6) an antibody comprising LCDR1, LCDR2, LCDR3, HCDR1, HCDR2, and HCDR3 represented by SEQ ID NOs: 84, 85, 91, 100, 102, and 108, respectively; or
(7) an antibody comprising LCDR1, LCDR2, LCDR3, HCDR1, HCDR2, HCDR3 represented by SEQ ID NO: 84, 85, 92, 100, 102, 109, respectively; or
(8) an antibody comprising LCDR1, LCDR2, LCDR3, HCDR1, HCDR2, and HCDR3 represented by SEQ ID NOs: 84, 85, 93, 100, 102, and 110, respectively; or
(9) an antibody comprising LCDR1, LCDR2, LCDR3, HCDR1, HCDR2, and HCDR3 represented by SEQ ID NOs: 84, 85, 94, 100, 102, and 111, respectively; or
(10) an antibody comprising LCDR1, LCDR2, LCDR3, HCDR1, HCDR2, and HCDR3 represented by SEQ ID NOs: 84, 85, 95, 100, 102, and 112, respectively; or
(11) an antibody comprising LCDR1, LCDR2, LCDR3, HCDR1, HCDR2, and HCDR3 represented by SEQ ID NOs: 84, 85, 96, 100, 102, and 113, respectively; or
(12) an antibody comprising LCDR1, LCDR2, LCDR3, HCDR1, HCDR2, and HCDR3 represented by SEQ ID NOs: 84, 85, 97, 100, 102, and 114, respectively; or
(13) an antibody comprising LCDR1, LCDR2, LCDR3, HCDR1, HCDR2, and HCDR3 represented by SEQ ID NOs: 84, 85, 98, 100, 102, and 115, respectively;
(14) an antibody, which is a variant of the antibody of any one of (1) to (13), and has the same or similar activity as the antibody of any one of (1) to (13).

In one embodiment, the antibody is selected from the group consisting of:
(1) an antibody comprising a light chain variable domain, wherein the light chain variable domain comprises an amino acid sequence represented by SEQ ID NO: 116, 117, 118, 119, 120, 121, 122, 123, 124, 125, 126, 127 or 128, or a nucleic acid sequence encoding the amino acid sequence, or a variant of any of the above sequences or an amino acid sequence having at least 80% identity with any of the above sequences or a nucleic acid sequence encoding the amino acid sequence;
(2) an antibody comprising a heavy chain variable domain comprising an amino acid sequence represented by SEQ ID NO: 129, 130, 131, 132, 133, 134, 135, 136, 137, 138, 139, 140 or 141, or a nucleic acid sequence encoding the amino acid sequence, or a variant of any of the above sequences, or an amino acid sequence having at least 80% identity with any of the above sequences or a nucleic acid sequence encoding the amino acid sequence;
(3) an antibody comprising the light chain variable domain of the antibody described in (1) and the heavy chain variable domain of the antibody described in (2).

In one embodiment, the antibody is selected from:
(1) an antibody comprising a heavy chain variable domain represented by SEQ ID NO: 129, and LCDR1, LCDR2, and LCDR3 represented by SEQ ID NOs: 84, 85, and 86, respectively; or an antibody comprising a light chain variable domain represented by SEQ ID NO: 116, and HCDR1, HCDR2, and HCDR3 represented by SEQ ID NOs: 99, 101, and 103, respectively; or
(2) an antibody comprising a heavy chain variable domain represented by SEQ ID NO: 130, and LCDR1, LCDR2, and LCDR3 represented by SEQ ID NOs: 84, 85, and 87, respectively; or an antibody comprising a light chain variable domain represented by SEQ ID NO: 117, and HCDR1, HCDR2, and HCDR3 represented by SEQ ID NOs: 99, 101, and 104, respectively; or
(3) an antibody comprising a heavy chain variable domain represented by SEQ ID NO: 131, and LCDR1, LCDR2, and LCDR3 represented by SEQ ID NOs: 84, 85, and 88, respectively; or an antibody comprising a light chain variable domain represented by SEQ ID NO: 118, and HCDR1, HCDR2, and HCDR3 represented by SEQ ID NOs: 99, 101, and 105, respectively; or
(4) an antibody comprising a heavy chain variable domain represented by SEQ ID NO: 132, and LCDR1, LCDR2, and LCDR3 represented by SEQ ID NOs: 84, 85, and 89, respectively; or an antibody comprising a light chain variable domain represented by SEQ ID NO: 119, and HCDR1, HCDR2, and HCDR3 represented by SEQ ID NOs: 99, 101, and 106, respectively; or
(5) an antibody comprising a heavy chain variable domain represented by SEQ ID NO: 133, and LCDR1, LCDR2, and LCDR3 represented by SEQ ID NOs: 84, 85, and 90, respectively; or an antibody comprising a light chain variable domain represented by SEQ ID NO: 120, and HCDR1, HCDR2, and HCDR3 represented by SEQ ID NOs: 99, 101, and 107, respectively; or
(6) an antibody comprising a heavy chain variable domain represented by SEQ ID NO: 134, and LCDR1, LCDR2, and LCDR3 represented by SEQ ID NOs: 84, 85, and 91, respectively; or an antibody comprising a light chain variable domain represented by SEQ ID NO: 121, and HCDR1, HCDR2, and HCDR3 represented by SEQ ID NOs: 100, 102, and 108, respectively; or
(7) an antibody comprising a heavy chain variable domain represented by SEQ ID NO: 135, and LCDR1, LCDR2, and LCDR3 represented by SEQ ID NOs: 84, 85, and 92, respectively; or an antibody comprising a light chain variable domain represented by SEQ ID NO: 122, and HCDR1, HCDR2, and HCDR3 represented by SEQ ID NOs: 100, 102, and 109, respectively; or
(8) an antibody comprising a heavy chain variable domain represented by SEQ ID NO: 136, and LCDR1, LCDR2, and LCDR3 represented by SEQ ID NOs: 84, 85, and 93, respectively; or an antibody comprising a light chain variable domain represented by SEQ ID NO: 123, and HCDR1, HCDR2, and HCDR3 represented by SEQ ID NOs: 100, 102, and 110, respectively; or
(9) an antibody comprising a heavy chain variable domain represented by SEQ ID NO: 137, and LCDR1, LCDR2, and LCDR3 represented by SEQ ID NOs: 84, 85, and 94, respectively; or an antibody comprising a light chain variable domain represented by SEQ ID NO: 124, and HCDR1, HCDR2, and HCDR3 represented by SEQ ID NOs: 100, 102, and 111, respectively; or
(10) an antibody comprising a heavy chain variable domain represented by SEQ ID NO: 138, and LCDR1, LCDR2, and LCDR3 represented by SEQ ID NOs: 84, 85, and 95, respectively; or an antibody comprising a light chain variable domain represented by SEQ ID NO: 125, and HCDR1, HCDR2, and HCDR3 represented by SEQ ID NOs: 100, 102, and 112, respectively; or
(11) an antibody comprising a heavy chain variable domain represented by SEQ ID NO: 139, and LCDR1, LCDR2, and LCDR3 represented by SEQ ID NOs: 84, 85, and 96, respectively; or an antibody comprising a light chain variable domain represented by SEQ ID NO: 126, and HCDR1, HCDR2, and HCDR3 represented by SEQ ID NOs: 100, 102, and 113, respectively; or
(12) an antibody comprising a heavy chain variable domain represented by SEQ ID NO: 140, and LCDR1, LCDR2, and LCDR3 represented by SEQ ID NOs: 84, 85, and 97, respectively; or an antibody comprising a light chain variable domain represented by SEQ ID NO: 127, and HCDR1, HCDR2, and HCDR3 represented by SEQ ID NOs: 100, 102, and 114, respectively; or
(13) an antibody comprising a heavy chain variable domain represented by SEQ ID NO: 141, and LCDR1, LCDR2, and LCDR3 represented by SEQ ID NOs: 84, 85, and 98, respectively; or an antibody comprising a light chain variable domain represented by SEQ ID NO: 128, and HCDR1, HCDR2, and HCDR3 represented by SEQ ID NOs: 100, 102, and 115, respectively.

In one embodiment, the antibody is selected from:
(1) an antibody, wherein the light chain variable domain and the heavy chain variable domain of the antibody have the amino acid sequences represented by SEQ ID NOs: 116 and 129, respectively, or amino acid sequences having 90%, 91%, 92%, 93%, 94%, 95%, 96%, 97%, 98%, or 99% identity with the above sequences, or nucleic acid sequences encoding the amino acid sequences;
(2) an antibody, wherein the light chain variable domain and the heavy chain variable domain of the antibody have the amino acid sequences represented by SEQ ID NOs: 117 and 130, respectively, or amino acid sequences having 90%, 91%, 92%, 93%, 94%, 95%, 96%, 97%, 98%, or 99% identity with the above sequences, or nucleic acid sequences encoding the amino acid sequences;
(3) an antibody, wherein the light chain variable domain and the heavy chain variable domain of the antibody have the amino acid sequences represented by SEQ ID NOs: 118 and 131, respectively, or amino acid sequences having 90%, 91%, 92%, 93%, 94%, 95%, 96%, 97%, 98%, or 99% identity with the above sequences, or nucleic acid sequences encoding the amino acid sequences;
(4) an antibody, wherein the light chain variable domain and the heavy chain variable domain of the antibody have the amino acid sequences represented by SEQ ID NOs: 119 and 132, respectively, or amino acid sequences having 90%, 91%, 92%, 93%, 94%, 95%, 96%, 97%, 98%, or 99% identity with the above sequences, or nucleic acid sequences encoding the amino acid sequences;
(5) an antibody, wherein the light chain variable domain and the heavy chain variable domain of the antibody have the amino acid sequences represented by SEQ ID NOs: 120 and 133, respectively, or amino acid sequences having 90%, 91%, 92%, 93%, 94%, 95%, 96%, 97%, 98%, or 99% identity with the above sequences, or nucleic acid sequences encoding the amino acid sequences;
(6) an antibody, wherein the light chain variable domain and the heavy chain variable domain of the antibody have the amino acid sequences represented by SEQ ID NOs: 121 and 134, respectively, or amino acid sequences having 90%, 91%, 92%, 93%, 94%, 95%, 96%, 97%, 98%, or 99% identity with the above sequences, or nucleic acid sequences encoding the above amino acid sequences;
(7) an antibody, wherein the light chain variable domain and the heavy chain variable domain of the antibody have the amino acid sequences represented by SEQ ID NOs: 122 and 135, respectively, or amino acid sequences having 90%, 91%, 92%, 93%, 94%, 95%, 96%, 97%, 98%, or 99% identity with the above sequences, or nucleic acid sequences encoding the above amino acid sequences;
(8) an antibody, wherein the light chain variable domain and the heavy chain variable domain of the antibody have the amino acid sequences represented by SEQ ID NOs: 123 and 136, respectively, or amino acid sequences having 90%, 91%, 92%, 93%, 94%, 95%, 96%, 97%, 98%, or 99% identity with the above sequences, or nucleic acid sequences encoding the above amino acid sequences;
(9) an antibody, wherein the light chain variable domain and the heavy chain variable domain of the antibody have the amino acid sequences represented by SEQ ID NOs: 124 and 137, respectively, or amino acid sequences having 90%, 91%, 92%, 93%, 94%, 95%, 96%, 97%, 98%, or 99% identity with the above sequences, or nucleic acid sequences encoding the amino acid sequences;
(10) an antibody, wherein the light chain variable domain and the heavy chain variable domain of the antibody have the amino acid sequences represented by SEQ ID NOs: 125 and 138, respectively, or amino acid sequences having 90%, 91%, 92%, 93%, 94%, 95%, 96%, 97%, 98%, or 99% identity with the above sequences, or nucleic acid sequences encoding the above amino acid sequences;
(11) an antibody, wherein the light chain variable domain and the heavy chain variable domain of the antibody have the amino acid sequences represented by SEQ ID NOs: 126 and 139, respectively, or amino acid sequences having 90%, 91%, 92%, 93%, 94%, 95%, 96%, 97%, 98%, or 99% identity with the above sequences, or nucleic acid sequences encoding the amino acid sequences;
(12) an antibody, wherein the light chain variable domain and the heavy chain variable domain of the antibody have the amino acid sequences represented by SEQ ID NOs: 127 and 140, respectively, or amino acid sequences having 90%, 91%, 92%, 93%, 94%, 95%, 96%, 97%, 98%, or 99% identity with the above sequences, or nucleic acid sequences encoding the amino acid sequences;
(13) an antibody, wherein the light chain variable domain and the heavy chain variable domain of the antibody have the amino acid sequences represented by SEQ ID NOs: 128 and 141, respectively, or amino acid sequences having 90%, 91%, 92%, 93%, 94%, 95%, 96%, 97%, 98%, or 99% identity with the above sequences, or nucleic acid sequences encoding the above amino acid sequences; or
(14) an antibody, which is a variant of the antibody of any one of (1) to (13), and has the same or similar activity as the antibody of any one of (1) to (13).

In one embodiment, the scFv of the antibody has an amino acid sequence represented by SEQ ID NO: 43, 68, 77, 81, 82 or 83, or an amino acid sequence having 90%, 91%, 92%, 93%, 94%, 95%, 96%, 97%, 98%, 99% identity with the above sequence, or a nucleic acid sequence encoding the amino acid sequence.

In one embodiment, the antibody is a full-length antibody, scFv, a single domain antibody, a Fab fragment, a Fab′ fragment, a Fv fragment, a F(ab′)2 fragment, a Fd fragment, a dAb fragment, a multifunctional antibody, a scFv-Fc antibody or an IgG4 antibody.

In one embodiment, the antibody binds to human or mouse MOG; and/or, the antibody binds to cells expressing human or mouse MOG.

The seventeenth aspect of the present application provides an immunoconjugate, which comprises: the antibody of the sixteenth aspect, and a functional molecule connected thereto.

The eighteenth aspect of the present application provides a chimeric polypeptide,
a) the binding domain of the chimeric polypeptide comprises the antibody of the sixteenth aspect; b) a receptor regulatory domain comprising one or more cleavage sites, the receptor regulatory domain comprising an extracellular domain and a transmembrane domain; and
c) an intracellular domain,
wherein the binding of the binding domain to MOG can induce the cleavage of the receptor regulatory domain, thereby releasing the intracellular domain.

The nineteenth aspect of the present application provides a biological material, which is any of the following:
1) a nucleic acid molecule encoding the chimeric polypeptide of any one of the first, second or eighteenth aspects, the molecule that recognizes the NKG2D ligand of the thirteenth aspect, the antibody of the sixteenth aspect, or the immunoconjugate of the seventeenth aspect;
2) a vector or an expression vector comprising the nucleic acid molecule of 1); or
3) a virus comprising the nucleic acid molecule of 1) or the vector or expression vector of 2).

It should be understood, that within the scope of the present application, the above-mentioned technical features and the technical features specifically described below (such as embodiments) of the present application can be combined with each other to form new or preferred technical solutions. Due to limited space, it will not be elaborated on them one by one here.

### INCORPORATION BY REFERENCE

All publications, patents, and patent applications mentioned herein are incorporated by reference to the same extent as if each individual publication, patent, or patent application was specifically and individually indicated to be incorporated by reference. If there is a conflict between a terminology herein and that in an incorporated reference, the terminology herein controls.

### BRIEF DESCRIPTION OF THE DRAWINGS

FIG. 1 shows that a chimeric polypeptide targeting GPC3, comprising the full-length EphrinB2 extracellular domain, or a truncation thereof, or a modified body thereof, regulates gene expression.

FIG. 2 shows that a chimeric polypeptide targeting MOG, comprising the full-length EphrinB2 extracellular domain or a truncation thereof regulates gene expression.

FIG. 3 shows that after co-incubation with MOG-positive cells, B7H3-CAR-T cells regulated by MOG-synE induced the expression of B7H3-CAR (FIG. 3A), effectively killed glioma cells (FIG. 3B), and released cytokine IL2 (FIG. 3C).

FIG. 4A shows the expression level of CD123 in AML cells; FIG. 4B shows the transcriptional activity triggered by the binding of the chimeric polypeptide targeting CD123.

FIG. 5 shows the expression levels of NKG2D ligands in tumor cells.

FIG. 6 shows that NKG2D-CAR-T cells regulated by the chimeric polypeptide CD123-synE or CD123-synE-del3 kill NKG2D ligand-positive tumor cells.

FIG. 7A shows that the expression of NKG2D ligands on tumor cells was detected; FIG. 7B shows that constitutive NKG2D-CAR-T cells can effectively kill NKG2D ligand-positive tumor cells, while regulatory NKG2D-CAR-T cells have almost no killing effect.

FIG. 8 shows that placing CD3Z-NKG2D-CAR under the regulation of CD123-synE or CD123-synE-del3 can improve the in vivo survival ability and anti-tumor effect of CAR-T cells.

FIG. 9 shows the expression of NKG2D-Ligand in resting NK cells from different donors detected by flow cytometry.

FIG. 10 shows that the expression of NKG2D-Ligand of NK cells was upregulated after NK cells were co-cultured with tumor cells or with UCAR-T for 24 hours.

FIG. 11 shows the killing effect of regulatory or constitutive NKG2D-UCAR T cells on NK cells.

FIG. 12 shows that when NK cells are present, regulatory NKG2D-UCAR-T cells can also kill tumor cells, and the killing effect becomes stronger as the co-incubation time prolongs.

FIG. 13 shows that when NK cells are present, regulatory NKG2D-UCAR-T cells significantly kill tumor cells. After co-incubation with tumor cells, the regulatory NKG2D-UCAR-T cells have a stronger killing effect on NK cells, and the longer the co-incubation time, the stronger the killing effect; and the regulatory NKG2D-UCAR-T cells can partially resist the killing effect of NK cells.

FIG. 14 shows that when NK cells are present, regulatory NKG2D-UCAR-T cells can effectively inhibit the growth of in situ tumors in mice.

FIG. 15 shows the binding of A1, A2, A3, A4, A5, A6, A7, A8, A9, A10, A11, A12, and A13 (in Fab form) antibodies to 293T-hMOG cells and 293T-mMOG cells.

FIG. 16 shows SDS-PAGE images of eukaryotic expressed antibodies A2, A6, A8, A9, A10, and A13 (in scFv-Fc form).

FIG. 17 shows the EC50 of antibodies A2, A6, A8, A9, A10, and A13 (scFv-Fc form) binding to MOG detected by ELISA.

FIG. 18 shows the EC50 of the binding of antibodies A2, A6, A8, A9, A10, and A13 (in scFv-Fc form) to 293T-hMOG and 293T-mMOG detected by FACs.

### DETAILED DESCRIPTION

The following description and examples provide a detailed explanation of embodiments of the disclosure. It is to be understood that this disclosure is not limited to the specific embodiments described herein, as such may vary. Those skilled in the art will recognize that there are many variations and modifications to the present application, which are within the scope thereof. Unless stated otherwise, any embodiment may be combined with any other embodiment.

As used herein, unless otherwise stated, some of the inventive embodiments herein contemplate numerical ranges. Various aspects of this application may be presented in a range format. It should be understood, that the description in range format is merely for convenience and brevity and should not be construed as an inflexible limitation on the scope of the application. Accordingly, the description of a range should be considered to have specifically disclosed all the possible subranges as well as individual numerical values within that range as if expressly written out. For example, description of a range such as 1 to 6 should be considered to have specifically disclosed sub-ranges such as 1 to 3, 1 to 4, 1 to 5, 2 to 4, 2 to 6, 3 to 6, etc., as well as individual numbers within that range, e.g., 1, 2, 3, 4, 5, and 6. This applies regardless of the breadth of the scope. When a range is presented, the range is inclusive of the range endpoints.

### Terms

Unless otherwise defined, all technical and scientific terms used herein have the same meanings as commonly understood by those skilled in the fields of gene therapy, biochemistry, genetics and molecular biology. All methods and materials similar or equivalent to those described herein can be used in the practice or testing of the present application, wherein appropriate methods and materials are described herein. All publications, applications, and other references mentioned herein are incorporated herein by reference in their entirety. In case of conflict, the present specification, comprising definitions, will control. In addition, unless otherwise specified, the materials, methods, and examples are illustrative only and not intended to be limiting.

Unless otherwise indicated, the practice of the present application will employ conventional techniques of cell biology, cell culture, molecular biology, transgenic biology, microbiology, recombinant DNA, and immunology, which are within the skill of the art. Such techniques are explained fully in references.

The term “NKG2D ligand (NKG2DL, NKG2D-L)” or “NKG2D-ligand”, i.e., a ligand that binds to NKG2D, comprises 8 polypeptides: MICA (Gene ID: 100507436), MICB (Gene ID: 4277), ULBP-1 (RAET1I, Gene ID: 80329), ULBP-2 (RAET1H, Gene ID: 80328), ULBP-3 (RAET1N, Gene ID: 79465), ULBP-4 (RAET1E, Gene ID: 135250), ULBP-5 (RAET1G, Gene ID: 353091) and ULBP-6 (RAET1L, Gene ID: 154064). The terms “anti-NKG2D ligand antibody”, “protein binding to NKG2D ligand”, “antibody to NKG2D ligand”, “antibody recognizing NKG2D ligand” refer to a polypeptide that binds to NKG2D ligand with sufficient affinity.

The term “NKG2D”, also known as KLRK1, Gene ID: 22914, is a C-type lectin family receptor on the surface of NK cells. It is also expressed in NKT cells, activated CD8+ T cells, CD4+ T cells and γδ+ T cells, etc. It is an important activating receptor for NK cells. In one embodiment, the NKG2D polypeptide comprises the sequence represented by SEQ ID NO:42.

The term “EphrinB2” refers to erythropoietin-producing hepatoma interactor B2, Gene ID: 1948, a cell surface ligand for the Eph receptor. Eph receptors are a class of tyrosine kinases that regulate cell migration, repulsion, and adhesion in neural, vascular, and epithelial development. In one embodiment, the EphrinB2 polypeptide comprises the sequence represented by SEQ ID NO:1. In one embodiment, the EphrinB2 polypeptide comprises the sequence represented by SEQ ID NO: 3, 4, 5 or 6.

The term “Jagged2” is also known as “JAG2, HJ2; SER2; LGMDR27”, Gene ID: 3714, and the protein encoded by this gene is one of several ligands that activate Notch and related receptors. Two transcript variants of this gene have been found encoding different isoforms.

The term “APLP2” is also known as “APPH; APPL2; CDEBP; APLP-2”, amyloid precursor like protein 2 (APLP2), is an APP (amyloid precursor protein) family member, including APLP1 (Gene ID: 333) and APLP2 (Gene ID: 334). This protein is ubiquitously expressed. This protein works synergistically with APP to mediate neuromuscular transmission, spatial learning, and synaptic plasticity. This protein has been implicated in the pathogenesis of Alzheimer's disease. Multiple alternatively spliced transcript variants encoding different isoforms have been identified.

The term “antibody” is used in the broadest sense herein and comprises various antibody structures, including but not limited to monoclonal antibodies, polyclonal antibodies, multi-specific antibodies (e.g., bispecific antibodies), domain antibodies, and antibody fragments thereof that can specifically bind to an antigen or antigenic determinant, provided they exhibit the required antigen-binding activity. The term “antibody fragments” refer to molecules other than intact antibodies that comprise a portion of an intact antibody that binds to the antigen to which the intact antibody binds. Examples of antibody fragments include, but are not limited to (i) Fab fragments consisting of the VL, VH, CL and CH1 domains, including Fab′ and Fab′-SH, (ii) Fd fragments consisting of the VH and CH1 domains, (iii) Fv fragments consisting of the VL and VH domains of a single antibody; (iv) dAb fragments consisting of a single variable region; (v) F(ab′)2 fragments, bivalent fragments comprising two linked Fab fragments; (vi) antigen-binding sites of single-chain Fv molecule; (vii) bispecific single-chain Fv dimers; (viii) “diabodies” or “triabodies”, multivalent or multi-specific fragments constructed by genetic fusion; and (ix) scFv genetically fused to the same or different antibodies.

The term “scFv” refers to a fusion protein comprising at least one antibody fragment comprising a light chain variable region and at least one antibody fragment comprising a heavy chain variable region, wherein the light and heavy chain variable regions are adjacent (e.g., connected by a synthetic linker, such as a short flexible polypeptide linker) and can be expressed as a single-chain polypeptide, and wherein the scFv retains the specificity of the intact antibody from which it is derived. Unless specified otherwise, as used herein, scFv can have the VL and VH variable regions in any order (e.g., relative to the N-terminus and C-terminus of the polypeptide), and scFv can comprise VL-linker-VH or can comprise VH-linker-VL. The antigen-binding function of an antibody may be performed by fragments of a naturally occurring antibody. These fragments are collectively referred to as “antigen binding units”. The term “antigen binding unit” also comprises any molecular structure comprising a polypeptide chain with a specific shape that is suitable for recognizing an epitope, wherein one or more non-covalent binding interactions stabilize the complex between the molecular structure and the epitope.

The term “variable region or variable domain” refers to the domain of an antibody heavy chain or light chain that is involved in antibody-antigen binding. The heavy chain variable domain (VH) and light chain variable domain (VL) of a natural antibody generally have similar structures, wherein each domain comprises four conserved FRs and three CDRs. A single VH or VL domain may confer antigen-binding specificity. Furthermore, antibodies that bind a specific antigen can be isolated by screening a library of complementary VL or VH domains, respectively, using a VH or VL domain from an antibody that binds to the antigen.

The terms “hypervariable region” or “complementarity determining region” or “CDR” refer to the regions of an antibody variable domain that exhibits sequence hypervariation, and/or forms structurally determined loops (“hypervariable loops”), and/or comprises residues that contact with the antigen (“antigenic contact site”). Typically, an antibody comprises six CDRs: three in VH (HCDR1, HCDR2, HCDR3) and three in VL (LCDR1, LCDR2, LCDR3).

The terms “Fc region” or “Fc” are used to define the C-terminal region of an immunoglobulin heavy chain that contains at least a portion of the constant region. The term comprises both native sequence Fc regions and variant Fc regions.

“Framework (FR)” refers to the variable domain residues other than the hypervariable region (CDR) residues. The FR of a variable domain typically consists of four FR domains: FR1, FR2, FR3 and FR4. In VH (or VL), CDR and FR sequences usually appear in the following order:
FR1-HCDR1 (LCDR1)-FR2-HCDR2 (LCDR2)-FR3-HCDR3 (LCDR3)-FR4.

Unless otherwise indicated, CDR residues and other residues in the variable domain (e.g., FR residues) are numbered herein according to Kabat numbering system et al., supra.

The term “natural antibody” refers to naturally occurring immunoglobulin molecules having a variety of structures. For example, natural IgG antibodies are heterotetrameric glycoproteins of approximately 150,000 daltons, consisting of two identical light chains and two identical heavy chains linked by disulfide bonds. From N-terminus to C-terminus, each heavy chain has a variable region (VH), also known as a variable heavy chain domain or a heavy chain variable domain, followed by three constant domains (CH1, CH2 and CH3). Similarly, from N-terminus to C-terminus, each light chain has a variable region (VL), also known as a variable light chain domain or a light chain variable domain, followed by a light chain constant (CL) domain. The light chains of antibodies can be classed into one of two types, known as kappa (κ) and lambda (λ), based on the amino acid sequence of their constant domains.

The terms “whole antibody”, “complete antibody”, “full-length antibody” and “intact antibody” are used interchangeably and refer to a complete full-length antibody having a structure substantially similar to a natural antibody structure or having a heavy chain of a Fc domain as defined herein or comprising an antigen-binding domain.

The term “single domain antibody (sdAb)” refers to a type of antibody that lacks the antibody light chain and only comprises the heavy chain variable domain. Because of its small molecular weight, it is also known as a “nanobody”.

The term “single domain antibody” or “mono-antibody” refers to an antibody comprising the entire or part of the heavy chain variable domain, or the entire or part of the light chain variable domain. The single domain antibody may be a human single domain antibody.

The term “monoclonal antibody” or “mAb” refers to an antibody obtained from a population of substantially homogeneous antibodies, i.e., the antibody molecules comprising the population are identical and/or bind to the same epitope, except for possible variant antibodies, e.g., those comprising naturally occurring mutations or arising during the preparation of the monoclonal antibody preparation, such variants generally exist in minor amounts. In contrast to polyclonal antibody preparations, which typically comprise different antibodies targeting different determinants (epitopes), each monoclonal antibody in a monoclonal antibody preparation targets a single determinant on the antigen. Thus, the modifier “monoclonal” indicates that the antibody is obtained from a population of essentially homologous antibodies, and is not considered to require the preparation of the antibody by any specific method. For example, it may be prepared using various techniques, including but not limited to: hybridoma method, recombinant DNA method, phage display method, and a method using a transgenic animal comprising all or part of human immunoglobulin gene loci.

The term “fully human antibody” or “human antibody” is an antibody that has an amino acid sequence corresponding to the amino acid sequence of an antibody produced by a human or human cell, or corresponding to the amino acid sequence obtained from an antibody of non-human origin that uses a human antibody library or other coding sequences of human antibody. The definition of fully human antibody explicitly excludes humanized antibodies comprising non-human antigen binding residues. A fully human antibody may be generated by phage display technology. A fully human antibody may be produced by an engineered strain and/or an engineered cell.

The term “antigen” or “Ag” refers to a substance that is recognized and specifically bound by an antibody or an antigen binding unit. Antigens may comprise peptides, proteins, glycoproteins, polysaccharides, lipids, portions thereof, or any combinations thereof. Antigens may comprise tumor antigens or pathogen antigens. “Antigen” may also refer to a molecule that elicits an immune response. This immune response may involve the production of antibodies or the activation of specific immunocompetent cells, or both. Those skilled in the art will appreciate that any macromolecule, comprising virtually all proteins or peptides, can serve as an antigen. The term “antigenic determinant site” is also known as “antigenic epitope” or “epitope” or “antigenic determinant”, and comprises any determinant or region capable of being bound by an antibody. An antigenic epitope is the region in an antigen that binds to an antibody targeting the antigen, comprising specific amino acids that are in direct contact with the antibody.

The terms “polypeptide,” “peptide,” and “protein” are used interchangeably herein to refer to polymers of amino acids of any length. The polymer may be linear, cyclic or branched, it may comprise modified amino acids, particularly conservatively modified amino acids, and it may be interrupted by non-amino acids. The term also comprises modified amino acid polymers, such as those modified by sulfation, glycosylation, lipidation, acetylation, phosphorylation, iodination, methylation, oxidation, proteolytic processing, prenylation, racemization, selenoylation, transfer-RNA-mediated amino acid addition such as arginylation, ubiquitination, or any other manipulation such as conjugation to a labeling component. The term “amino acid” refers to natural and/or unnatural or synthetic amino acids, comprising amino acids having optical isomers, as well as amino acid analogs and peptidomimetics.

The term “peptide-drug conjugate (PDC)” mainly consists of three parts: peptide, linker and cytotoxicity payload, which is used for targeted therapy. For example, payloads comprise adriamycin, paclitaxel, MTX, and camptothecin.

The term “conservative modification” or “conservative sequence modification” refers to amino acid modifications that do not significantly affect or change the required activity or properties of a peptide containing the amino acid sequence, comprising amino acid substitutions, insertions and deletions. Modifications can be introduced into the antibodies of the present application by standard techniques known in the art, such as site-directed mutagenesis, or PCR-mediated mutagenesis. For example, families of amino acid residues with similar side chains have been defined in the art, as shown in Table 1.

TABLE 1
Families of amino acid residues with similar side chains

| | |
|---|---|
| Amino acids with | Lys(K), Arg(R), His(H) |
| alkaline side chains | |
| Amino acids with acidic | Asp(D), Glu(E) |
| side chains | |
| Amino acids with polar | Asn(N), Ser(S), Thr(T), Tyr(Y), Cys(C), |
| neutral side chains | Trp(W), Met(M), Gln(Q) |
| Amino acids with non- | Gly(G), Ala(A), Val(V), Leu(L), Ile(I), |
| polar side chains | Pro(P), Phe(F) |
| Amino acids with β- | Thr(T), Val(V), Ile(I) |
| branched side chains | |
| Amino acids with | Tyr(Y), Phe(F), Trp(W) |
| aromatic side chains | |

Thus, one or more amino acid residues in the CDR domain or in the framework region of the antibody of the present application can be replaced with other amino acid residues of the same side chain family, and the altered antibody (variant antibody) can be tested for retained function. Non-conservative substitutions entail exchanging a member of one of these groups for a member of another group. One substitutional variant involves substituting one or more hypervariable region residues of a parent antibody (e.g., a humanized or human antibody). Typically, the resulting variants selected for further study will have changes (e.g., improvements) in certain biological properties (e.g., increased affinity, or reduced immunogenicity) relative to the parent antibody and/or will substantially retain certain biological properties of the parent antibody. An exemplary substitutional variant is an affinity-matured antibody, for example, routinely prepared using phage display-based affinity maturation techniques such as those described herein. Briefly, one or more CDR residues are mutated and the variant antibodies are displayed on phage and screened for a particular biological activity (e.g., binding affinity).

Changes (e.g., substitutions) can be introduced in the CDR regions, for example, to improve antibody affinity. Such changes can be made in CDR “hot spots”, i.e., residues encoded by codons that are mutated at high frequency during the somatic hypermaturation process, and/or residues involved in antigen unbinding, and the VH or VL of the resulting variant is tested for binding affinity. In some embodiments of affinity maturation, diversity is introduced into the variable genes selected for maturation by any of a variety of methods (e.g., error-prone PCR, chain shuffling, or oligonucleotide-directed mutagenesis). A secondary library is then generated. The library is then screened to identify any antibody variants having the desired affinity. Another method to introduce diversity includes the CDR-directed approach, wherein several CDR residues (e.g., 4-6 residues simultaneously) are randomized.

Substitutions, insertions, or deletions may occur within one or more CDRs, provided that such changes do not significantly reduce the ability of the antibody to bind antigen. For example, conservative changes (e.g., conservative modifications described herein) that do not significantly reduce binding affinity can be made in the CDRs. Such changes may be outside of the residues that contact the antigen in the CDR. In the VH and VL sequences of the variant provided above, each CDR may be unchanged, or comprise no more than one, two or three amino acid substitutions.

The term “transplant immune rejection” refers to the immune response of a host to a foreign transplant, recognized as an “alien component.” This response results in the transplant being attacked, destroyed, and cleared by the host's immune system after an allogeneic tissue, organ, or cell transplant is introduced. The present application provides a cell for resisting transplant immune rejection and a method for resisting transplant rejection.

The term “graft” refers to a biological material or preparation derived from an individual other than the host and intended for implantation into the host. The graft may be of any animal origin, such as mammalian origin, preferably human. The graft can be from the host, such as cells from the host that are cultured in vitro or transformed and then implanted into the host again. The graft can be from another allogeneic individual, such as cells from another person that are cultured in vitro or transformed and implanted into a host. The graft may be from a xenogeneic individual, such as an organ from another species (such as mouse, pig, monkey) transplanted into a human. Xenografts include, but are not limited to, vascularized xenografts, partially vascularized xenografts, non-vascularized xenografts, xenodressings, xenobandages, and xenostructures.

The term “autologous” refers to originating from the same organism. For example, a sample can be removed from a subject (e.g., cells), processed, and returned to the subject (e.g., patient) at a later time. Autologous procedures are distinguished from allogeneic procedures in which the donor and recipient are different subjects. The term “autologous transplantation” comprises any procedure involving the transplantation, implantation or infusion of cells, tissues or organs into a recipient wherein the subject and the donor are the same individual. Transplantation of cells, organs and/or tissues described herein may be used for autologous transplantation into humans. Autologous transplantation includes, but is not limited to, vascularized autologous transplantation, partially vascularized autologous transplantation, non-vascularized autologous transplantation, autologous dressings, autologous bandages, and autologous structures.

The term “allogeneic transplantation” comprises any procedure involving the transplantation, implantation or infusion of cells, tissues or organs into a subject, wherein the subject and the donor are different individuals of the same species. Transplantation of the cells, organs and/or tissues described herein may be used for allogeneic transplantation into humans. Allogeneic transplantation includes, but is not limited to, vascularized allogeneic transplantation, partially vascularized allogeneic transplantation, non-vascularized allogeneic transplantation, allodressings, allobandages, and allogenic structures.

The term “cell” refers to a cell of human or non-human origin or of animal origin.

The term “host” refers to the subject into which the graft is transplanted. For example, it may be an individual, such as a human, into whom exogenous cells are implanted.

The term “individual” refers to any animal, such as a mammal or marsupial. The individuals of the present application include, but are not limited to, humans, non-human primates (e.g., rhesus monkeys or other types of macaques), mice, pigs, horses, donkeys, cows, sheep, rats, and any kind of poultry.

The term “immune cell” refers to cells that participate in immune response and produce immune effectors, such as T cells, B cells, natural killer (NK) cells, natural killer T (NKT) cells, dendritic cells, CIK cells, macrophages, mast cells, etc. For example, the immune cells are T cells, NK cells, and NKT cells. For example, the T cells can be autologous T cells, xenogeneic T cells, or allogeneic T cells. For example, the NK cells can be autologous NK cells or allogeneic NK cells. The term “CIK cells”, i.e. cytokine-induced killer (CIK) cells, is a new type of immune active cells. CIK cells have strong proliferation ability, strong cytotoxic effect and certain immune characteristics. Since the cells express two membrane protein molecules, CD3 and CD56, they are also called NK cell-like T lymphocytes (NKT cells). They have both the powerful anti-tumor activity of T lymphocytes and the non-MHC-restricted tumor-killing advantages of NK cells. For example, immune cells are obtained by sorting donor peripheral blood mononuclear cells (PBMCs).

The term “engineered cells having immune effector cell function” refers to cells or cell lines that do not have immune effects and that have acquired immune cell function after being artificially modified or stimulated by stimulants. For example, 293T cells are engineered to have the function of immune effector cells; stem cells are induced in vitro to differentiate into immune cells.

The “T cells” described herein can be PBMC, bone marrow, lymph node tissue, umbilical cord blood, thymus tissue, and natural T cells obtained from infection sites, ascites, pleural effusion, spleen tissue, and tumor tissue. T cells may be a cell population with specific phenotypic characteristics obtained through sorting, etc., or a mixed cell population with different phenotypic characteristics. T cells can be cells comprising at least one of the following subpopulations of T cells: stem cell-like memory T cells (Tscm cells), central memory T cells (Tcm), effector T cells (Tef, Teff), regulatory T cells (tregs) and/or effector memory T cells (Tem). For example, T cells can be a certain subtype of T cells, such as αβ T cells or γδ T cells. For example, T cells can be obtained from blood collected from an individual using any technique known to those of skill in the art, such as Ficoll™ separation and/or apheresis. For example, PBMCs are collected by apheresis and then T cells are obtained by screening. T cells can be any type of T cells and can be at any developmental stage, including but not limited to CD4+/CD8+ double positive T cells, CD4+ helper T cells, such as Th1 and Th2 cells, CD8+ T cells (such as cytotoxic T cells), tumor infiltrating cells, memory T cells, naive T cells, etc. The T cells may be CD8+ T cells or CD4+ T cells. For example, cells are obtained from an individual's circulating blood by apheresis. Apheresis products typically comprise lymphocytes, comprising T cells, monocytes, granulocytes, B cells, other nucleated white blood cells, red blood cells, and platelets. For example, cells collected by apheresis can be washed to remove plasma molecules and the cells can be placed in an appropriate buffer or culture medium for subsequent processing steps. For example, the T cells can be obtained from a healthy donor, or from an individual diagnosed with cancer. The conventional method for preparing CAR-T in this field is to use PBMC cells collected by apheresis, and then continue to culture the obtained T cells after activation with magnetic beads of anti-CD3 and CD28 antibodies, and obtain CAR-T cells after lentivirus infection.

The “NK cells” described in this application include primary NK cells or NK cell lines. For example, NK cells can be isolated from PBMC of healthy donors by sorting CD56-positive cells; or NK cells can be obtained by removing other types of cells from PBMCs by negative screening. Most of the NK cells isolated from PBMC of healthy donors are resting NK cells, which do not undergo autonomous proliferation. They become activated after being stimulated by target cells such as tumor cells or by IL2, IL15 and/or IL18. Activated NK cells proliferate rapidly and have enhanced cell killing function. Resting NK cells can hardly detect of NKG2D ligand expression. After co-incubation with tumor cells, NKG2D ligand expression was detected on the NK cell membrane. After co-incubation with UCAR-T cells (B2M knockout, TCR/B2M knockout, TCR/B2M/FAS knockout), NKG2D ligand expression was detected on the NK cell membrane.

The terms “activation” and “stimulation” are used interchangeably to refer to the process by which cells transition from a quiescent state to an active state. The process may comprise responses to antigen, phenotypic or genetic changes in migration and/or functional activity state. For example, the term “activation” may refer to the step-wise process of T cell activation. The activation process is jointly regulated by the first stimulation signal and the co-stimulation signal. T cell activation is a dynamic process, and its duration and degree of activation are affected by external stimulation. “T cell stimulation” or “T cell activation” refers to the state of a T cell that is stimulated to induce detectable cell proliferation, cytokine production, and/or detectable effector function. Using CD3/CD28 magnetic beads, in vitro antigen stimulation or in vivo antigen stimulation will affect the degree and duration of T cell activation. For example, the engineered T cells are activated after co-culture with tumor cells containing specific target antigens or virus infection.

The term “peripheral blood mononuclear cell (PBMC)” refers to cells with a single nucleus in peripheral blood, comprising lymphocytes, monocytes, and the like. PBMCs are obtained by density-based cell separation methods, such as by lysing red blood cells or without lysing red blood cells and by gradient centrifugation of peripheral blood or aliquots or leukapheresis samples using Percoll Ficoll.

The term “engineered” or “engineering” refers to the application of the principles and methods of cell biology and molecular biology to change the genetic material within the cell or obtain cell products at the cellular level or organelle level through some engineering means. For example, “engineered” refers to one or more changes in a nucleic acid (e.g., a nucleic acid within the genome of an organism). “Engineering” may refer to the alteration, addition and/or deletion of genes. Engineered cells may also refer to cells that have added, deleted and/or altered genes.

The “low expression” mentioned in the present application means that the protein and/or RNA level expressed by the target gene in the engineered cells is lower than the expression level before the cell engineering treatment. For example, low expression of B2M, TCR, FAS, NKG2A or NKG2D ligand means that the expression of B2M, TCR, FAS, NKG2A or NKG2D ligand in the cell is reduced by at least 1%, at least 5%, at least 10%, at least 20%, at least 30%, at least 40%, at least 50%, at least 60%, at least 70%, at least 80%, at least 90%, at least 95%, at least 99% or 100%. The expression or amount of a specific protein in a cell can be determined by any suitable method known in the art, such as ELISA, immunohistochemistry, immunoblotting or flow cytometry using specific antibodies.

The term “MHC” is the histocompatibility complex, referred to in human cells as HLA antigens, which mediate rejection by T cells that respond to histocompatibility antigens on the surface of the implanted tissue. The term “B2M” is beta-2 microglobulin, also known as B2M, which is the light chain of the MHC class I molecule.

The term “recombinant T cell receptor (recombinant TCR)” comprises chimeric receptors derived from one or more TCR subunits. For example, a recombinant TCR comprises at least a portion of the extracellular domain of a TCR subunit, the transmembrane domain and the TCR intracellular domain, wherein the TCR subunit portion is operatively linked to an antigen binding domain. For example, the TCR subunits in the recombinant TCR are derived from CD3ζ, CD3ε, CD3γ, CD3δ, TCRα, TCRβ, TCRγ and/or TCRδ subunits. For example, a recombinant TCR can be integrated into a TCR/CD3 complex expressed on a T cell. For example, a recombinant TCR comprises the constant domains and intracellular domains of TCRα and TCRβ subunits, and the subunit constant domains are effectively linked to the antigen binding domains. For example, a recombinant TCR comprises the constant domains and intracellular domains of TCRγ and TCRδ subunits, and the subunit constant domains are effectively linked to the antigen binding domains. For example, the recombinant TCR comprises a CD3ζ, CD3ε, CD3γ or CD3δ subunit, and the extracellular domain of the subunit is effectively linked to an antigen binding domain.

The term “gene editing” refers to a genetic engineering technique that uses site-specific nucleases to insert, knock out, modify or replace DNA at a specific location in the genome to change the DNA sequence. Gene knockout technology using nucleases comprises CRISPR/Cas9 technology, ZFN technology, TALE technology and TALE-CRISPR/Cas9 technology, Base Editor technology, guide editing technology and/or homing nuclease technology. A guide sequence (gRNA) is a polynucleotide sequence that has sufficient complementarity to a target polynucleotide sequence so as to hybridize to the target sequence, and the gRNA is capable of directing sequence-specific binding of the CRISPR complex to the target sequence. Whenever the sequence of gRNA is involved in this application, it can be a targeted DNA sequence, or it can be a complete Cas9 guide sequence formed by the ribonucleotides corresponding to the DNA and crRNA and TracrRNA. gRNA is used to guide, bind or recognize Cas enzymes. For example, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. For example, CRISPR technology is used to construct endogenous TCR/B2M/FAS knockout or endogenous TCR/B2M knockout engineered cells. The gRNA sequences targeting TCR, B2M, and FAS are represented by SEQ ID NOs: 142, 143, and 144, respectively.

The term “transfection”, “transduction”, or “transformation” refers to the introduction of exogenous nucleic acid into eukaryotic cells, which can be achieved by various means known in the art. For example, calcium phosphate-DNA coprecipitation, DEAE-dextran-mediated transfection, polybrene-mediated transfection, electroporation, microinjection, liposome fusion, lipofection, protoplast fusion, retroviral infection, and biolistics.

The terms “nucleic acid molecule encoding” “encoding DNA sequence,” and “encoding DNA” refer to the sequence or order of a deoxyribonucleotide along a deoxyribonucleic acid chain. For example, a nucleic acid sequence encodes an amino acid sequence. When referring to a nucleotide sequence, a “sequence” may comprise DNA or RNA and may be single-stranded or double-stranded.

The term “homology” or “identity” refers to the subunit sequence identity between two polymer molecules (e.g., between two nucleic acid molecules such as two DNA molecules or two RNA molecules, or between two polypeptide molecules). The term “substantial identity” or “substantial homology” refers to polypeptides or nucleic acid molecules that exhibit at least about 50% homology or identity with a reference amino acid sequence or nucleic acid sequence. For example, such a sequence has at least about 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology or identity with the amino acid or nucleic acid sequence used for comparison. Sequence identity can be measured by using sequence analysis software (e.g., BLAST, BESTFIT, GAP or PILEUP/PRETTYBOX programs).

The term “expression vector” refers to a vector comprising a recombinant polynucleotide which comprises an expression regulatory sequence operatively linked to a nucleotide sequence to be expressed. The expression vector comprises sufficient cis-acting elements for expression; other elements for expression can be provided by the host cell or in vitro expression system. Expression vectors comprise plasmids, viruses (e.g., lentiviruses, retroviruses, adenoviruses, and adeno-associated viruses).

The term “vector” is a composition that comprises an isolated nucleic acid and can be used to deliver the isolated nucleic acid to the interior of a cell, for example, including but not limited to: linear polynucleotides, polynucleotides associated with ions or amphiphilic compounds, plasmids, and viruses. For example, autonomously replicating plasmids or viruses are included. It also comprises non-plasmid and non-viral compounds that facilitate transfer of nucleic acids into cells, such as polylysine compounds, liposomes, and the like.

The term “isolated” means separated from cellular or other components with which the polynucleotide, peptide, polypeptide, protein, antibody or fragment thereof is normally associated in nature. As will be appreciated by those skilled in the art, a non-naturally occurring polynucleotide, peptide, polypeptide, protein, antibody or fragment thereof need not be “isolated” to distinguish it from its naturally occurring counterpart. In addition, a “concentrated,” “isolated,” or “diluted” polynucleotide, peptide, polypeptide, protein, antibody or fragment thereof can be distinguished from its naturally occurring counterpart because the concentration or number of molecules per volume is greater (“concentrated”) or less (“diluted”) than that of its naturally occurring counterpart.

The term “exogenous” refers to a nucleic acid molecule or polypeptide, cell, tissue, etc. that is not endogenously expressed in the organism itself, or whose expression level is insufficient to achieve its function when overexpressed.

The term “endogenous” refers to a nucleic acid molecule, polypeptide, etc. that originates from the organism itself.

The term “chimeric receptor” refers to a fusion molecule formed by connecting DNA fragments or corresponding cDNAs of proteins from different sources using genetic recombination technology, comprising an extracellular domain, a transmembrane domain and an intracellular domain. Chimeric receptors include, but are not limited to, chimeric antigen receptors (CARs) and recombinant TCR receptors.

The term “chimeric antigen receptor” (CAR) comprises an extracellular antigen binding domain, a transmembrane domain, and an intracellular signaling domain. The intracellular signaling domain comprises a functional signaling domain of a stimulatory molecule and/or a co-stimulatory molecule. In one aspect, the stimulatory molecule is a zeta(ζ) chain that binds to a T cell receptor complex; in one aspect, the cytoplasmic signaling domain further comprises a functional signaling domain of one or more co-stimulatory molecules, such as 4-1BB (i.e., CD137), CD27, and/or CD28. For example, groups of polypeptides are linked to each other.

The term “primary signaling domain” or “primary signaling region” regulates the initial activation of the TCR complex in a stimulatory manner. In one aspect, the primary signaling is triggered by, for example, binding of a TCR/CD3 complex to a peptide-loaded MHC molecule, thereby mediating a T cell response (including but not limited to, proliferation, activation, differentiation, etc.). The primary signaling domain, which acts in a stimulatory manner, may comprise an immunoreceptor tyrosine-based activation motif or an ITAM signaling motif. For example, the segments of the primary signaling domain comprising ITAMs include, but are not limited to, sequences derived from TCRε, FcRγ, FcRβ, CD3γ, CD3δ, CD3ε, CD3ζ, CD5, CD22, CD79a, CD79b, CD278 (also known as “ICOS”), and CD66d. For example, the intracellular signaling domain in the CAR of the present application comprises an intracellular signaling sequence, such as the primary signaling domain of CD3ζ.

The term “signaling domain” or “signaling transduction domain” refers to a functional portion of a protein that acts by transmitting information within a cell to regulate the activity of the cell via a certain signaling pathway by generating second messengers or by acting as an effector in response to such messengers. The intracellular signaling domain may comprise the entire intracellular portion of the molecule, or the entire native intracellular signaling domain, or a functional fragment or derivative thereof.

The term “co-stimulatory molecule” or “costimulatory molecule” refers to a molecule that binds to a cell-stimulatory signaling molecule, such as TCR/CD3, and in combination results in T cell proliferation and/or up-regulation or down-regulation of key molecules. It is a cognate binding partner on T cells that specifically binds to co-stimulatory ligands and mediates the co-stimulatory response of T cells, including but not limited to cell proliferation. Co-stimulatory molecules are non-antigen receptor cell surface molecules or their ligands required for an effective immune response. Co-stimulatory molecules include, but are not limited to, MHC class I molecules, BTLA and Toll ligand receptors, as well as OX40, CD2, CD27, CD28, CDS, ICAM-1, LFA-1 (CD11a/CD18), and 4-1BB (CD137).

The intracellular signaling domain (or region) can be selected from any one or more of the intracellular co-stimulatory domains of the following polypeptides: CD27, CD28, TNFRSF9, TNFRSF4, TNFRSF8, TNFRSF14, TNFRSF18, CD40LG, ICOS, ITGB2, CD2, CD7, KLRC2, HAVCR1, LGALS9, and CD83.

The term “CD3ζ (also known as CD3Zeta)” comprises the protein provided by GenBank Accession No. BAG36664.1, or equivalent residues from non-human species such as mouse, rodent, monkey, ape, etc. In this application, “CD3ζ” is used interchangeably with “CD3z” and “CD3Z”.

The term “effective amount” or “therapeutically effective amount” refers to a dose sufficient to prevent or treat a disease (tumor or cancer) in an individual. The effective dosage for therapeutic or prophylactic use will depend on the stage and severity of the disease being treated, the age, weight and general health of the subject and the judgment of the prescribing physician. The size of the dose will also depend on the active substance chosen, the method of administration, the timing and frequency of administration, the existence, nature and extent of adverse side effects that may accompany the administration of the specific active substance, and the desired physiological effect. According to the judgment of the prescribing physician or a person skilled in the art, one or more rounds, or multiple administrations of the engineered cells of the present application may be required. By way of example and not limitation of the present application, an exemplary dose of engineered cells may be at least one million cells (1×10⁶ cells/dose).

The engineered cells (e.g., T, NK and/or NKT cells) provided by the present application can be used to treat, prevent or improve autoimmune diseases or inflammatory diseases, in particular inflammatory diseases associated with autoimmune diseases, such as arthritis (e.g., rheumatoid arthritis, chronic progressive arthritis and deforming arthritis) and rheumatic diseases, comprising inflammatory conditions and rheumatic diseases involving bone loss, inflammatory pain, spondyloarthropathies (including ankylosing spondylitis), Reiter's syndrome, reactive arthritis, psoriatic arthritis, juvenile idiopathic arthritis and enteropathic arthritis, enthesitis, hypersensitivity (including airway hypersensitivity and skin hypersensitivity) and allergies. The engineered T cells provided in the present application are used to treat and prevent autoimmune hematological disorders (comprising, for example, hemolytic anemia, aplastic anemia, pure red cell anemia and idiopathic thrombocytopenia), systemic lupus erythematosus (SLE), lupus nephritis, inflammatory muscle disease (dermatomyositis), periodontitis, polychondritis, scleroderma, Wegener's granulomatosis, dermatomyositis, chronic active hepatitis, myasthenia gravis, psoriasis, Stevens Johnson syndrome, spontaneous sprue, autoimmune inflammatory bowel disease (comprising, for example, ulcerative colitis, Crohn's disease and irritable bowel syndrome), endocrine eye diseases, Graves' disease, sarcoidosis, multiple sclerosis, systemic sclerosis, fibrotic diseases, primary biliary cirrhosis, juvenile diabetes (type I diabetes), uveitis, keratoconjunctivitis sicca and vernal keratoconjunctivitis, interstitial pulmonary fibrosis, periprosthetic osteolysis, Glomerulonephritis (with and without nephrotic syndrome, comprising, for example, idiopathic nephrotic syndrome or minimal change nephropathy), multiple myeloma, other types of tumors, inflammatory diseases of the skin and cornea, myositis, loosening of bone implants, metabolic disorders (such as obesity, atherosclerosis and other cardiovascular diseases, comprising dilated cardiomyopathy, myocarditis, type II diabetes and dyslipidemia) and autoimmune thyroid disease (including Hashimoto's thyroiditis), primary vasculitis of small and medium vessels, large vessel vasculitis comprising giant cell arteritis, hidradenitis suppurativa, neuromyelitis optica, Sjogren's syndrome, Behcet's disease, atopic and contact dermatitis, bronchiolitis, inflammatory muscle disease, autoimmune peripheral neuropathies, immune kidney, liver and thyroid diseases, inflammation and atherosclerosis, autoinflammatory febrile syndrome, immune hematological disorders, and bullous diseases of the skin and mucous membranes.

The engineered cells (e.g., T, NK and/or NKT cells) provided in the present application can be used to treat, prevent or improve asthma, bronchitis, bronchiolitis, idiopathic interstitial pneumonia, pneumoconiosis, emphysema and other obstructive or inflammatory diseases of the airways.

Provided herein is an engineered cell (e.g., T, NK, and/or NKT cells), wherein the engineered cell expresses a chimeric receptor that recognizes a NKG2D ligand, and optionally, the engineered cell further comprises a DAP10 polypeptide or a fragment thereof. The engineered cell can constitutively express a chimeric receptor that recognizes a NKG2D ligand (e.g., comprising a constitutive promoter). The engineered cell can inducibly express a chimeric receptor that recognizes a NKG2D ligand (e.g., comprising an inducible promoter). The engineered cells can resist the killing of the host immune cells (e.g., T, NK and/or NKT cells). In the presence of host immune cells (e.g., T, NK and/or NKT cells), the engineered cells have a longer survival time and/or a higher transplant survival rate compared to reference cells (e.g., cells that do not express NKG2D-CAR). The engineered cells can be autologous cells or allogeneic cells. The engineered cells can increase the persistence and/or transplantation survival rate of another immune cell in the presence of host immune cells (e.g., T, NK and/or NKT cells). The engineered cell and/or another immune cell may be cells from the same individual or may be allogeneic cells.

Provided herein is an engineered cell (e.g., T, NK, and/or NKT cells), which expresses: a chimeric polypeptide and a chimeric receptor that recognizes a NKG2D ligand, wherein the chimeric polypeptide can regulate the transcriptional activity of the chimeric receptor; optionally, the engineered cell further comprises a DAP10 polypeptide or a fragment thereof. The engineered cells can resist the killing of the host immune cells (e.g., T, NK and/or NKT cells). In the presence of host immune cells (e.g., T, NK and/or NKT cells), the engineered cells have a longer survival time and/or a higher transplantation survival rate compared to reference cells (e.g., cells that do not express NKG2D-CAR). The engineered cells can be autologous cells or allogeneic cells. The engineered cells can increase the persistence and/or transplantation survival rate of another immune cell in the presence of host immune cells (e.g., T, NK, NKT cells). The engineered cell and/or another immune cell may be cells from the same individual or may be allogeneic cells.

The engineered cells of the present application (e.g., T, NK and/or NKT cells) can be administered as the sole active ingredient or combined with other agents such as immunosuppressants or immunomodulators or other anti-inflammatory agents or other cytotoxic agents or anti-cancer agents (e.g., as adjuvants or in combination with them), for example, to treat or prevent diseases related to immune disorders. For example, the engineered cells of the present application can be used in combination with the following agents: DMARDs, such as gold salts, sulfasalazine, antimalarial drugs, methotrexate, D-penicillamine, azathioprine, mycophenolic acid, tacrolimus, sirolimus, minocycline, leflunomide, glucocorticoids; calcineurin inhibitors, such as cyclosporine A or FK 506; regulators of lymphocyte recirculation, such as FTY720 and FTY720 analogs; mTOR inhibitors, such as rapamycin, 40-O-(2-hydroxyethyl)-rapamycin, CCI779, ABT578, AP23573 or TAFA-93; ascomycins with immunosuppressive properties, such as ABT-281, ASM981, etc.; corticosteroids; cyclophosphamide; azathioprine; leflunomide; mizoribine; mycophenolate mofetil; 15-deoxyspergualin or its immunosuppressive homologs, analogs or derivatives; immunosuppressive monoclonal antibodies, for example, monoclonal antibodies targeting leukocyte receptors, such as MHC, CD2, CD3, CD4, CD7, CD8, CD25, CD28, CD40, CD45, CD58, CD80, CD86 or their ligands; antibodies that bind to immune checkpoint inhibitors, for example, the checkpoint inhibitors comprise: (a) one or more antagonists of checkpoint molecules, which comprise PD-1, PDL-1, TIM-3, TIGIT, LAG-3, CTLA-4, 2B4, 4-1BB, 4-1BBL, A2aR, BATE, BTLA, CD39, CD47, CD73, CD94, CD96, CD160, CD200, CD200R, CD274, CEACAM1, CSF-1R, Foxp1, GARP, HVEM, IDO, EDO, TDO, LAIR-1, MICA/B, NR4A2, MAFB, OCT-2, RARα (retinoic acid receptor alpha), TLR3, VISTA, NKG2A/HLA-E or inhibitory KIR, (b) one or more of atezolizumab, avelumab, durvalumab, ipilimumab, IPH4102, IPH43, IPH33, lirimumab, monalizumab, nivolumab, pembrolizumab and derivatives or functional equivalents thereof, or (c) at least one of atezolizumab, nivolumab and pembrolizumab; TOLL-like receptor agonist poly(I:C); sorafenib, regorafenib; chemotherapeutic agents: cyclophosphamide, fludarabine, albumin paclitaxel or any combination thereof, 5-fluorouracil or a prodrug or an active metabolite thereof, oxaliplatin or a prodrug or an active metabolite thereof, a taxanes, or any combination thereof; olaparib; gemcitabine; other immunomodulatory compounds. The engineered cells may also be used in combination with whole body or localized radiation.

The engineered cells (e.g., T, NK and/or NKT cells) of the present application may also express one or any combination of the following polypeptides: 1) another chimeric receptor that recognizes tumor antigens and/or pathogen antigens, for example, another chimeric receptor is CAR and/or recombinant TCR; 2) cytokines, such as IL7 and/or CCL21, IL7+CCL19, sPD-1, IFNβ, IL4R extracellular domain+IL21R intracellular domain, IL-12, TGF-β receptor extracellular domain+IL-2R intracellular domain, RUNX3, RUNX3+IL15 (or IL18 or IL21), IL21, IL21+CCL19, IL15, IL18, CD11a fusion protein, HLA-E-B2M chimeric protein, CXCR4, or any combination thereof; 3) antibodies that bind to immune checkpoint inhibitors, for example, the checkpoint inhibitors comprise: (a) one or more antagonists of checkpoint molecules, which comprise PD-1, PDL-1, TIM-3, TIGIT, LAG-3, CTLA-4, 2B4, 4-1BB, 4-1BBL, A2aR, BATE, BTLA, CD39, CD47, CD73, CD94, CD96, CD160, CD200, CD200R, CD274, CEACAM1, CSF-1R, Foxp1, GARP, HVEM, IDO, EDO, TDO, LAIR-1, MICA/B, NR4A2, MAFB, OCT-2, RARα (retinoic acid receptor alpha), TLR3, VISTA, NKG2A/HLA-E or inhibitory KIR; (b) one or more of atezolizumab, avelumab, durvalumab, ipilimumab, IPH4102, IPH43, IPH33, lirimumab, monalizumab, nivolumab, pembrolizumab and derivatives or functional equivalents thereof, or (c) at least one of atezolizumab, nivolumab and pembrolizumab.

Methods for obtaining suitable engineered cells may include selection based on specific markers. For example, markers may comprise GFP, resistance genes, cell surface markers, or endogenous tags. Any endogenous marker may be used to select cells. Suitable cell selection techniques include flow cytometry and/or magnetic columns. The selected engineered cells are then infused into the subject. The selected engineered cells can also be expanded to large numbers. The selected engineered cells can be expanded prior to infusion. The effective amount of engineered cells used to treat a subject can vary depending on cell viability and the efficiency with which the cells were genetically modified (e.g., the efficiency with which the transgene is integrated into one or more cells, or the expression level of a protein encoded by the transgene). The viability (e.g., proliferation capacity) of genetically modified cells and the efficiency of transgene integration can be used to determine the effective amount of engineered cells to be administered to a subject. The increase in cell viability after genetic modification can reduce the administration dose of the engineered cells. The increase in the efficiency of transgene integration into one or more cells can reduce the administration dose of the engineered cells. The therapeutically effective amount of the engineered cells can be determined by determining the cell viability as a function of time. A therapeutically effective amount of engineered cells can be determined by determining a function of changes in the efficiency of integration of the transgene into one or more cells relative to time-related variables (e.g., cell culture time, electroporation time, cell stimulation time). Therapeutically effective cells can be a population of cells comprising about 30% to about 100% expressing the NKG2D-CAR or chimeric polypeptide on the cell surface. In one embodiment, therapeutically effective cells may express about 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9% to greater than about 99.9% of the NKG2D-CAR or chimeric polypeptide on the cell surface as measured by flow cytometry.

The engineered cells (e.g., T, NK and/or NKT cells) provided by the present application can be used to treat, prevent or improve any tumor disease, comprising acute lymphocytic carcinoma, acute myeloid leukemia, alveolar rhabdomyosarcoma, bladder cancer, bone cancer, brain cancer (e.g., medulloblastoma), breast cancer, anal cancer, anal canal cancer or anorectal cancer, eye cancer, intrahepatic bile duct cancer, joint cancer, neck cancer, gallbladder cancer, pleural cancer, nasal cancer, nasal cavity cancer or middle ear cancer, oral cancer, vulvar cancer, chronic lymphocytic leukemia (CLL), chronic myeloid cancer, colon cancer, esophageal cancer, cervical cancer, fibrosarcoma, gastrointestinal carcinoid, head and neck cancer (e.g., head and neck squamous cell carcinoma), Hodgkin's Lymphoma, hypopharyngeal cancer, kidney cancer, laryngeal cancer, leukemia, liver cancer, lung cancer (such as non-small cell lung cancer), lymphoma, malignant mesothelioma, mast cell tumor, melanoma, multiple myeloma, nasopharyngeal cancer, non-Hodgkin's lymphoma, B-chronic lymphocytic leukemia, B cell-precursor acute lymphoblastic leukemia (B-ALL), B cell-precursor acute lymphoblastic leukemia (BCP-ALL), B cell lymphoma, acute lymphoblastic leukemia (ALL), Burkitt's lymphoma, ovarian cancer, pancreatic cancer, pharyngeal cancer, prostate cancer, rectal cancer, kidney cancer, skin cancer, small intestine cancer, soft tissue cancer, stomach cancer, testicular cancer, thyroid cancer, and ureter cancer. Preferably, the tumor is characterized by high expression of NKG2D-ligands, such as, solid tumors with high expression of NKG2D-ligands, CD123-positive hematological tumors, CLL1-positive hematological tumors, B7H3 positive brain tissue tumors, and B7H3 positive gliomas.

The term “tumor antigen” refers to an antigen that emerges or is overexpressed during the development and progression of a hyperproliferative disease. Hyperproliferative disorders are called cancers or tumors. Tumor antigens comprise solid tumor antigens and hematological tumor antigens (or liquid tumor antigens).

The tumor antigens of the present application include, but are not limited to: thyroid stimulating hormone receptor (TSHR); CD171; CS-1 (CS1); C-type lectin-like molecule-1; ganglioside GD3; Tn antigen; CD19; CD20; CD22; CD30; CD70; CD123; CD138; CD33; CD44; CD44v7/8; CD38; CD44v6; B7H3 (CD276), B7H6; KIT (CD117); IL-13Ra; IL-11Ra; PSCA; PSMA; CEA; NY-ESO-1; HIV-1 Gag; MART-1; gp100; tyrosinase; mesothelin; EpCAM; proteinase serine 21 (PRSS21); VEGFR, VEGFR2; Lewis (Y) antigen; CD24; PDGFR-β; SSEA-4; MUC1; MUC6; EGFR; EGFR2; ERBB3; ERBB4; EGFRvIII; neural cell adhesion molecule (NCAM); carbonic anhydrase IX (CAIX); LMP2; EphA2; fucosyl GM1; sialyl Lewis adhesion molecule (sLe); ganglioside GM3; TGS5; high molecular weight melanoma associated antigen (HMWMAA); OAcGD2; folate receptor; TEM1/CD248; TEM7R; Claudin 6; Claudin18.2; Claudin18.1; ASGPR1; CDH16; 5T4; 8H9; αvβ6; BCMA; CA9; kappa light chain; CSPG4; EGP2, EGP40; FAP; FAR; FBP; embryonic AchR; HLA-A1; HLA-A2; MAGEA1, MAGE3; KDR; MCSP; NKG2D ligand; PSC1; ROR1; Sp17; SURVIVIN; TAG72; TEM1; fibronectin; tenascin; carcinoembryonic variant of tumor necrosis; GPRC5D; CXORF61; CD97; CD179a; ALK; polysialic acid; PLAC1; Hexasaccharide Moiety of Globo H (GloboH); breast differentiation antigen (NY-BR-1); uroplakin 2 (UPK2); hepatitis A virus cellular receptor 1 (HAVCR1); adrenergic receptor β3 (ADRB3); pannexin 3 (PANX3); G protein-coupled receptor 20 (GPR20); lymphocyte antigen 6 complex locus K9 (LY6K); olfactory receptor 51E2 (OR51E2); TCRγ alternate reading frame protein (TARP); WT1; ETV6-AML; SPA17; XAGE1; Tie2; MAD-CT-1; MAD-CT-2; Fos-related antigen 1; p53 mutant; human telomerase reverse transcriptase (hTERT); sarcoma translocation breakpoints; melanoma inhibitor of apoptosis (ML-IAP); ERG (transmembrane protease serine 2 (TMPRSS2) ETS fusion gene); N-acetylglucosaminyltransferase V (NA17); paired box protein Pax-3 (PAX3); androgen receptor; cyclin B1; MYCN; RhoC; CYP1B1; CCCTC-binding factor (zinc finger protein)-like (BORIS); SART3; PAX5; OYTES1; LCK; AKAP-4; SSX2; CD79a; CD79b; CD72; LAIR1; FCAR; LILRA2; CD300LF; CLEC12A; BST2; EMR2; lymphocyte antigen 75 (LY75); glypican-3 (GPC3); FCRL5; FcRH5, or immunoglobulin lambda-like polypeptide 1 (IGLL1). Preferably, the tumor antigen is CD123, CLL1, CD19, CD20, CD22, CD38, BCMA, GPRC5D, B7H3, GPC3, Claudin 6, Claudin18.2, FAP, Mesothelin, NKG2D-ligand, NKG2A, or CD94. The term “pathogen antigen” is selected from: antigens of viruses, bacteria, fungi, protozoa, or parasites; viral antigens are selected from: cytomegalovirus antigens, Epstein-Barr virus antigens, human immunodeficiency virus antigens, or influenza virus antigens.

The terms “recognize,” “bind,” and “target” are used interchangeably and refer to selective binding to a target antigen. For example, recognizing a target cell means binding to a target antigen (e.g., a target molecule) on the target cell. In one embodiment, the engineered cell recognizes the target cell, that is, the engineered cell targets the target cell. For example, engineered cells can inhibit or kill target cells by binding to target antigens on the target cells. For example, engineered cells expressing chimeric receptors that recognize NKG2D ligands can inhibit or kill target cells (e.g., T, NK and/or NKT cells) by recognizing or targeting NKG2D ligands on target cells (e.g., T, NK and/or NKT cells). In one embodiment, NKG2D-CAR-T cells inhibit or kill host NK cells by recognizing or targeting NKG2D ligands on host NK cells (e.g., allogeneic NK cells).

The present application provides isolated nucleic acids encoding chimeric receptors that recognize NKG2D ligands, chimeric polypeptides that have transcriptional regulation activity, chimeric receptors that recognize tumor antigens, antibodies that recognize MOG, or fragments thereof, vectors and expression vectors comprising the nucleic acids, and engineered cells (also referred to as host cells) comprising the nucleic acids and vectors. The nucleic acid can be in intact cells, in a cell lysate, or in a partially purified or substantially purified form. For example, this application comprises: the nucleic acids of GPC3-synE, GPC3-synE-del1, GPC3-synE-del2, GPC3-synE-del3, GPC3-synE-del23, GPC3-synE-EGFn (n=1, 2, 3, 4), MOG-synE, MOG-synE-del3, CD123-synE, CD123-synE-del3, CLDN18.2-synE, GPRC5D-synE, CLL1-synE, B7H3-CAR-MOG-synE, B7H3-CAR-MOG-synE-del3, ZNKG2D-CAR-CD123-synE, ZBB-NKG2D-CAR-CD123-synE, ZNKG2D-CAR-DAP10-CD123-synE, ZNKG2D-CAR-CD123-synE-del3, ZBB-NKG2D-CAR-CD123-synE-del3 and ZNKG2D-CAR-DAP10-CD123-synE-del3, and engineered cells comprising the nucleic acids.

The expression of the polynucleic acid of the chimeric receptor recognizing a NKG2D ligand and/or the chimeric polypeptide having transcriptional regulation activity can be controlled by one or more promoters. The promoter can be a ubiquitous promoter, a constitutive promoter (an unregulated promoter that allows continuous transcription of the associated gene), a tissue-specific promoter, or an inducible promoter (a promoter with regulated transcriptional activity that can dramatically and rapidly regulate transcription of the chimeric receptor gene when stimulated by inducing conditions).

The nucleic acid of the present application can be obtained using standard molecular biology techniques, for example, by standard PCR amplification or cDNA cloning technology, to obtain cDNA encoding the light and heavy chains of the antibody or encoding the VH and VL segments. For antibodies obtained from an immunoglobulin gene library (e.g., using phage display technology), one or more nucleic acids encoding the antibody can be recovered from the library. Methods for introducing exogenous nucleic acids into host cells are generally known in the art and may vary depending on the host cell used.

A gene encoding a chimeric receptor recognizing a NKG2D ligand and/or a gene encoding a chimeric polypeptide having transcriptional regulation activity may be introduced into the cell, and optionally, a gene encoding a DAP10 polypeptide or a fragment thereof may also be introduced. For example, the gene is introduced into engineered cells (e.g., T, NK and/or NKT cells). When introduced into a cell, the gene can be a complementary DNA (cDNA) segment, a copy of the messenger RNA (mRNA), or the gene itself residing in its original genomic DNA domain (with or without introns). The gene encoding the chimeric receptor recognizing the NKG2D ligand and the gene encoding the chimeric polypeptide having transcription regulating activity can be introduced into the cell simultaneously; optionally, the gene encoding the DAP10 polypeptide or a fragment thereof is also introduced. The gene encoding the chimeric receptor recognizing the NKG2D ligand and the gene encoding the chimeric polypeptide having transcription regulating activity may be located in the same vector. Optionally, the vector further comprises a gene encoding the DAP10 polypeptide or a fragment thereof. In one embodiment, the chimeric polypeptide can modulate the expression of a chimeric receptor that recognizes a NKG2D ligand and a DAP10 polypeptide.

Compared with reference cells (e.g., cells that do not express NKG2D-CAR), engineered cells comprising constitutively expressed NKG2D-CAR or engineered cells comprising regulatedly expressed NKG2D-CAR have enhanced survival and expansion capabilities during in vivo and in vitro culture.

The DNA encoding the transgene may also be designed to include a reporter gene, so that the presence of the transgene or its expression product can be detected by activation of the reporter gene. Any reporter gene can be used. Cells in which the reporter gene is activated, i.e., cells containing the transgene, can be selected in cell culture.

Expression of the exogenously introduced gene can be verified by expression assays (e.g., qPCR) or by measuring RNA levels. Expression level may also be indicative of copy number. For example, if the expression level is very high, this indicates that the exogenously introduced gene may have been integrated into the genome in multiple copies. Alternatively, the exogenously introduced gene is integrated in a highly transcribed domain, for example, near a highly expressed promoter. Expression can also be verified by measuring protein levels, such as by immunoblotting.

The chimeric receptor (e.g., NKG2D-CAR) that recognizes NKG2D ligands provided in the present application refers to a fusion molecule formed by connecting DNA fragments or cDNAs or peptide segments corresponding to proteins from different sources, comprising an extracellular domain, a transmembrane domain, and an intracellular domain; optionally, the extracellular domain is directly connected to the transmembrane domain or connected through a hinge. For example, the chimeric receptor includes but not limited to: a chimeric antigen receptor (CAR), a recombinant TCR receptor. NKG2D-CAR can be expressed under the regulation of the chimeric polypeptide provided in the present application. Notch polypeptides or polypeptides derived therefrom well known to those skilled in the art can be used to regulate the chimeric receptors of the present application that recognize NKG2D ligands.

The chimeric receptor that recognizes NKG2D ligands comprises: 1) an extracellular domain, which can be selected from: the extracellular domain of NKG2D polypeptide, or an antibody that recognizes NKG2D ligands or a fragment thereof; 2) a transmembrane domain, which can be selected from: the transmembrane domains of TCRα chain, TCRβ chain, TCRγ chain, TCRδ chain, CD3ζ subunit, CD3ε subunit, CD3γ subunit, CD3δ subunit, BAFFR, CEACAM1, CD100 (SEMA4D), CD103, CD11a, CD11b, CD11c, CD11d, CD137 (4-1BB), CD16, CD160, CD18 (CD11a, LFA-1), CD160 (BY55), CD162 (SELPLG), CD19, CD2, CD22, CD226(DNAM1), CD229 (Ly9), CD244(SLAMF4, 2B4), CD27, CD278(ICOS), CD28, CD29, CD33, CD37, CD4, CD40, CD45, CD49a, CD49D, CD49f, CD5, CD64, CD8, CD80, CD84, CD86, CD9, CD96(Tactile), CD134, CD154, CRTAM, GITR, HLA-E, HLA-F, HLA-G, HVEM (LIGHTR), IA4, IL2Rβ, IL2Rγ, IL7Rα, ITGA1, ITGA4, ITGA6, ITGAD, ITGAE, ITGAL, ITGAM, ITGAX, ITGB1, ITGB2, ITGB7, KIRDS2, LTBR, NKp80(KLRF1), NKp44, NKp30, NKp46, NKG2D, NKG2C, OCIL, OX40, PAG/Cbp, PSGL1, SLAM (SLAMF1, CD150, IPO-3), SLAMF6 (NTB-A, Ly108), SLAMF7, BLAME(SLAMF8), TNFR2, VLA1, VLA-6, cadherin and/or collagen; 3) an intracellular domain, which can be selected from: the intracellular signaling domain of TCRε, FcRγ, FcRβ, CD3γ, CD3δ, CD3ε, CD3ζ, CD5, CD22, CD79a, CD79b, CD278, CD66d, NKG2D, CD28 or CD137, or any combination thereof.

The chimeric receptor recognizing the NKG2D ligand can be selected from: 1) comprising the full-length NKG2D polypeptide or a fragment thereof; 2) comprising the full-length NKG2D polypeptide and an immunoreceptor tyrosine-based activation motif; 3) comprising the full-length NKG2D polypeptide and an ITAM signaling motif; 4) comprising the full-length NKG2D polypeptide, and the intracellular signaling domain of TCRε, FcRγ, FcRβ, CD3γ, CD3δ, CD3ε, CD3ζ, CD5, CD22, CD79a, CD79b, CD278, CD66d, CD28 or CD137, or any combination thereof; 5) comprising the full-length NKG2D polypeptide and the intracellular signaling domain of CD3ζ; 6) comprising the full-length NKG2D polypeptide, the intracellular signaling domain of CD28 and the intracellular signaling domain of CD3ζ; 7) comprising the full-length NKG2D polypeptide, the intracellular signaling domain of CD137 and the intracellular signaling domain of CD3 ζ; 8) comprising the full-length NKG2D polypeptide, the intracellular signaling domain of CD28, the intracellular signaling domain of CD137 and the intracellular signaling domain of CD3 ζ; 9) comprising the extracellular domain of the NKG2D polypeptide, the transmembrane domain of the NKG2D polypeptide, and an immunoreceptor tyrosine-based activation motif; 10) comprising the extracellular domain of the NKG2D polypeptide, the transmembrane domain of the NKG2D polypeptide, and a ITAM signaling motif; 11) comprising the extracellular domain of the NKG2D polypeptide, the transmembrane domain of the NKG2D polypeptide, and the intracellular signaling domain of TCRε, FcRγ, FcRβ, CD3γ, CD3δ, CD3ε, CD3ζ, CD5, CD22, CD79a, CD79b, CD278, CD66d, CD28 or CD137, or any combination thereof, 12) comprising the extracellular domain of the NKG2D polypeptide, the transmembrane domain of the NKG2D polypeptide, and the intracellular signaling domain of CD3ζ; 13) comprising the extracellular domain of the NKG2D polypeptide, the transmembrane domain of the NKG2D polypeptide, the intracellular signaling domain of CD28, and the intracellular signaling domain of CD3ζ; 14) comprising the extracellular domain of the NKG2D polypeptide, the transmembrane domain of the NKG2D polypeptide, the intracellular signaling domain of CD137, and the intracellular signaling domain of CD3ζ; 15) comprising the extracellular domain of the NKG2D polypeptide, the transmembrane domain of the NKG2D polypeptide, the intracellular signaling domain of CD28, the intracellular signaling domain of CD137, and the intracellular signaling domain of CD3ζ; 16) comprising the extracellular domain of the NKG2D polypeptide, the transmembrane domain of CD28 or CD8, and an immunoreceptor tyrosine-based activation motif; 17) comprising the extracellular domain of a NKG2D polypeptide, the transmembrane domain of CD28 or CD8, and a signaling motif of ITAM; 18) comprising the extracellular domain of the NKG2D polypeptide, the transmembrane domain of CD28 or CD8, and the intracellular signaling domain of TCRε, FcRγ, FcRβ, CD3γ, CD3δ, CD3ε, CD3ζ, CD5, CD22, CD79a, CD79b, CD278, CD66d, CD28 or CD137, or any combination thereof; 19) comprising the extracellular domain of the NKG2D polypeptide, the transmembrane domain of CD28 or CD8, and the intracellular signaling domain of CD3ζ; 20) comprising the extracellular domain of the NKG2D polypeptide, the transmembrane domain of CD28 or CD8, the intracellular signaling domain of CD28, and the intracellular signaling domain of CD3ζ; 21) comprising the extracellular domain of the NKG2D polypeptide, the transmembrane domain of CD28 or CD8, the intracellular signaling domain of CD137, and the intracellular signaling domain of CD3ζ; 22) comprising the extracellular domain of the NKG2D polypeptide, the transmembrane domain of CD28 or CD8, the intracellular signaling domain of CD28, the intracellular signaling domain of CD137 and the intracellular signaling domain of CD3ζ.

In one embodiment, the chimeric receptor recognizing the NKG2D ligand comprises a fragment having at least 60%, 65%, 70%, 75%, 80%, 85%, 90%, 95%, 96%, 97%, 98%, 99% or 100% homology or identity with the amino acid sequence represented by SEQ ID NO:51, 52, 53 and/or 54, and/or may optionally comprise at most 1, 2, 3, 4, 5 or more amino acid residues being replaced by different amino acid residues.

In some examples, the chimeric receptor that recognizes the NKG2D ligand may have one or any combination of the following properties: 1) binding to recombinant human NKG2D ligands (MICA, MICB, ULBP-1, ULBP-2, ULBP-3, ULBP-4, ULBP-5 and/or ULBP-6); 2) binding to endogenous NKG2D ligands (MICA, MICB, ULBP-1, ULBP-2, ULBP-3, ULBP-4, ULBP-5 and/or ULBP-6) on the surface of tumor cells; 3) binding to endogenous NKG2D ligands (MICA, MICB, ULBP-1, ULBP-2, ULBP-3, ULBP-4, ULBP-5 and/or ULBP-6) on the surface of tumor cells; 4) binding to endogenous NKG2D ligands (MICA, MICB, ULBP-1, ULBP-2, ULBP-3, ULBP-4, ULBP-5 and/or ULBP-6) on the surface of human AML cells; 5) binding to endogenous NKG2D ligands (MICA, MICB, ULBP-1, ULBP-2, ULBP-3, ULBP-4, ULBP-5 and/or ULBP-6) selected from the surface of THP1, KG-1, or Molm 13 cells; 6) binding to endogenous NKG2D ligands (MICA, MICB, ULBP-1, ULBP-2, ULBP-3, ULBP-4, ULBP-5 and/or ULBP-6) on the surface of human NK cells; 7) binding to endogenous NKG2D ligands (MICA, MICB, ULBP-1, ULBP-2, ULBP-3, ULBP-4, ULBP-5 and/or ULBP-6) on the surface of activated human NK cells; 8) binding to endogenous NKG2D ligands (MICA, MICB, ULBP-1, ULBP-2, ULBP-3, ULBP-4, ULBP-5 and/or ULBP-6) on the surface of human NK cells after co-incubation with tumor cells; 9) binding to endogenous NKG2D ligands (MICA, MICB, ULBP-1, ULBP-2, ULBP-3, ULBP-4, ULBP-5 and/or ULBP-6) on the surface of human NK cells after co-incubation with T cells; 10) binding to endogenous NKG2D ligands (MICA, MICB, ULBP-1, ULBP-2, ULBP-3, ULBP-4, ULBP-5 and/or ULBP-6) on the surface of human NK cells after co-incubation with T cells (endogenous B2M knockout T cells).

The engineered cells that recognize NKG2D ligands may comprise one or more exogenous genes. The one or more exogenous genes can express a chimeric receptor that binds at least one NKG2D ligand (e.g., MICA, MICB, ULBP-1, ULBP-2, ULBP-3, ULBP-4, ULBP-5, and/or ULBP-6). An engineered cell that recognizes a NKG2D ligand may also comprise one or more chimeric receptors, or it may comprise a single chimeric receptor and a secondary engineered receptor. Engineered cells that recognize NKG2D ligands can encode a suicide gene transgene. Suicide genes can induce the elimination of engineered cells that recognize NKG2D ligands. A suicide gene is any gene that induces apoptosis in the CAR immune responding cell. The suicide gene can be encoded within the viral vector along with a chimeric receptor that recognizes the NKG2D ligand.

The engineered cells of the present application that constitutively express chimeric receptors that recognize NKG2D ligands (e.g., NKG2D-CAR) or regulatory express chimeric receptors that recognize NKG2D ligands (e.g., NKG2D-CAR) may also have one or any combination of the following characteristics: 1) low expression or no expression of endogenous NKG2D ligands; 2) low expression or no expression of endogenous B2M/TCR; 3) low expression or no expression of endogenous B2M/TCR/NKG2D ligands; 4) low expression or no expression of endogenous B2M/TCR/FAS; 5) low expression or no expression of endogenous B2M/TCR/FAS/NKG2D ligands; 6) knocking out endogenous immune checkpoints, for example, a polynucleotide fragment encoding NKG2D-CAR is inserted into an endogenous immune checkpoint gene so that the gene is interrupted, for example, the immune checkpoints are PD-1, PD-L1, or CTLA-4. For example, gene editing techniques are used to knock out endogenous FAS, B2M, TCR and/or NKG2D ligands. For example, gene editing techniques are used to knock out endogenous FAS, B2M, TCR and/or NKG2D ligands. For example, CRISPR technology is used to knock out endogenous FAS, B2M, TCR and/or NKG2D ligands.

In one embodiment, the engineered cells that recognize NKG2D ligands comprise: ZNKG2D-CAR, ZBB-NKG2D-CAR, ZNKG2D-CAR-DAP10 and/or NKG2D-28Z-CAR polypeptides; optionally, the engineered cells also comprise a gRNA fragment targeting B2M/TCR or B2M/TCR/FAS. Immune cells that recognize NKG2D ligands may comprise: fragments having at least 60%, 65%, 70%, 75%, 80%, 85%, 90%, 95%, 96%, 97%, 98%, 99% or 100% homology or identity with the amino acid sequences represented by SEQ ID NO: 51, 52, 53 and/or 54, and/or may optionally comprise at most 1, 2, 3, 4, 5 or more amino acid residues being replaced by different amino acid residues; optionally, the immune cells also comprise a gRNA sequence (e.g., SEQ ID NO: 142, 143 and/or 144).

The engineered cells of the present application constitutively or regulatory expressing NKG2D-CAR also express one or any combination of the following polypeptides: 1) another chimeric receptor that recognizes tumor antigens and/or pathogen antigens, for example, the other chimeric receptor is CAR and/or recombinant TCR; 2) cytokines; 3) antibodies that bind to immune checkpoint inhibitors.

The engineered cells of the present application constitutively or regulatory expressing NKG2D-CAR are used in combination with one or more of the following anticancer agents: TOLL-like receptor agonist poly(I:C); sorafenib; regorafenib; chemotherapeutic agents: cyclophosphamide, fludarabine, albumin paclitaxel or any combination thereof; 5-fluorouracil or prodrug or active metabolite thereof, oxaliplatin or prodrug or active metabolite thereof, taxanes, or any combination thereof; olaparib; gemcitabine; mTOR inhibitor: rapamycin.

The engineered cells of the present application regulatory expressing NKG2D-CAR also express one any combination of the following polypeptides: 1) polypeptides for regulating NKG2D-CAR, for example, synNOTCH polypeptides and polypeptides derived therefrom; 2) polypeptides having binding-triggered transcriptional regulatory activity, for example, chimeric polypeptides provided herein; 3) chimeric polypeptides whose receptor regulatory domains comprise the EphrinB2 extracellular domain, and its modified bodies (for example, EphrinB2-(EGF-like domain) n, n=1-4), or its truncations (for example, EphrinB2EC-del1, EphrinB2EC-del2, EphrinB2EC-del3 or EphrinB2EC-del23); 4) polypeptides that bind to tumor antigens to trigger transcriptional regulatory activity, for example, tumor antigens are ALPPL2, ALPI, Axl, B7H3, BCMA, CD4117, CD123, CD171, CD179a, CD19, CD213A2, CD20, CD22, CD24, CD246, CD272, CD30, CD33, CD38, CD44v6, CD46, CD7, CD71, CD94, CD97, CEA, Claudin18.2, CLDN6, CLECL1, CLL1, cMet, CS-1, EGFR, EGFRVIII, ELF2M, EpCAM, EphA2, FAP, FCRH5, FLT3, GD2, GD3, GM3, GPC3, GPRC5D, HER2 (ERBB2), IGLL1, IL 11Ra, IL13Ra2, Mesothelin, MUC1, NCAM, NKG2D-Ligand, PAP, PDGFR-b, PRSS21, PSCA, PSMA, ROR1, SIRPa, SSEA-4, TAG72, TEM1/CD248, TEM7R, TSHR, VEGFR2 or WT1; 5) polypeptides that bind to pathogen antigens to trigger transcriptional regulatory activity; 6) polypeptides that bind to tissue-specific antigens to trigger transcriptional regulatory activity, for example, brain tissue-specific marker MOG, liver tissue-specific marker ASGR1, prostate tissue marker PSA, etc. or any combination thereof; 7) polypeptides that bind to hematological tumor antigens to trigger transcriptional regulatory activity, such as CD123, CLL1, GPRC5D, FcRH5, CD38, BCMA, CD19, or CD20, etc.; 8) polypeptides that bind to solid tumor antigens to trigger transcriptional regulatory activity, such as, B7H3, GPC3, Claudin 6. Claudin18.2, FAP, Mesothelin, NKG2D ligand, NKG2A, or CD94; 9) another chimeric receptor that recognizes tumor antigens and/or pathogen antigens, for example, the other chimeric receptor is CAR and/or recombinant TCR.

In one embodiment, the engineered cells of the present application regulatory expressing NKG2D-CAR comprise: ZNKG2D-CAR, ZBB-NKG2D-CAR, ZNKG2D-CAR-DAP10 and/or NKG2D-28Z-CAR polypeptide fragments; and also comprise chimeric polypeptides that specifically bind to antigens such as Mesothelin, FAP, Claudin18.2, CLL1, CD19, GPC3, WT1, HER2, EGFR, EGFRVIII, BCMA, CD123, MOG, B7H3, NKG2A or FcRH5, and regulate the expression of NKG2D-CAR. Preferably, the engineered cells further comprise a gRNA fragment targeting B2M/TCR or B2M/TCR/FAS.

The engineered cells of the present application regulatory expressing NKG2D-CAR may comprise: ZNKG2D-CAR-CD123-synE (e.g., SEQ ID NO: 55), ZBB-NKG2D-CAR-CD123-synE (e.g., SEQ ID NO: 56), ZNKG2D-CAR-DAP10-CD123-synE (e.g., SEQ ID NO: 57), NKG2D-28Z-CAR-CD123-synE (e.g., comprising SEQ ID NOs: 48 and 54), ZNKG2D-CAR-CD123-synE-del3 (e.g., comprising SEQ ID NOs: 49 and 52), ZBB-NKG2D-CAR-CD123-synE-del3 (e.g., comprising SEQ ID NOs: 49 and 51), ZNKG2D-CAR-DAP10-CD123-synE-del3 (e.g., comprising SEQ ID NOs: 49 and 53), and NKG2D-28Z-CAR-CD123-synE-del3 (e.g., comprising SEQ ID NOs: 49 and 54) fragments. Preferably, the engineered cells further comprise a gRNA fragment targeting B2M/TCR or B2M/TCR/FAS. More preferably, the engineered cell further comprises a gRNA sequence (e.g., SEQ ID NO: 142, 143 and/or 144).

The engineered cells of the present application regulatory expressing NKG2D-CAR may comprise: a fragment having at least 60%, 65%, 70%, 75%, 80%, 85%, 90%, 95%, 96%, 97%, 98%, 99% or 100% homology or identity with the amino acid sequence represented by SEQ ID NO: 51, 52, 53 and/or 54, and/or may optionally comprise at most 1, 2, 3, 4, 5 or more amino acid residues being replaced by different amino acid residues; also comprise a fragment having at least 60%, 65%, 70%, 75%, 80%, 85%, 90%, 95%, 96%, 97%, 98%, 99% or 100% homology or identity with the amino acid sequence represented by SEQ ID NO: 44, 45, 46, 47, 48 and/or 49, and/or may optionally comprise at most 1, 2, 3, 4, 5 or more amino acid residues being replaced by different amino acid residues. Preferably, the nucleic acid fragments encoding the chimeric polypeptide having transcriptional regulatory activity and the NKG2D-CAR regulated by the chimeric polypeptide are located in the same expression vector. Preferably, the engineered cells further comprise a gRNA fragment targeting B2M/TCR or B2M/TCR/FAS. More preferably, the engineered cell further comprises a gRNA sequence (e.g., SEQ ID NO: 142, 143 and/or 144).

The engineered cells of the present application regulatory expressing NKG2D-CAR may comprise: a fragment having at least 60%, 65%, 70%, 75%, 80%, 85%, 90%, 95%, 96%, 97%, 98%, 99% or 100% homology or identity with a polynucleotide sequence represented by SEQ ID NO: 55, 56 and/or 57 or an amino acid sequence encoded thereby, and/or may optionally comprise at most 1, 2, 3, 4, 5 or more amino acid residues being replaced by different amino acid residues. Preferably, the engineered cells further comprise a gRNA fragment targeting B2M/TCR or B2M/TCR/FAS. More preferably, the engineered cell further comprises a gRNA sequence (e.g., SEQ ID NO: 142, 143 and/or 144). The constitutively expressed NKG2D-CAR-T cells or regulatory expressed NKG2D-CAR-T cells of the present application may also have one or any combination of the following characteristics: 1) low expression or no expression of endogenous NKG2D ligands; 2) low expression or no expression of endogenous B2M/TCR; 3) low expression or no expression of endogenous B2M/TCR/NKG2D ligands; 4) low expression or no expression of endogenous B2M/TCR/FAS; 5) low expression or no expression of endogenous B2M/TCR/FAS/NKG2D ligands; 6) knockout of endogenous immune checkpoints, for example, a polynucleotide fragment encoding NKG2D-CAR is inserted into an endogenous immune checkpoint gene so that the gene is interrupted, for example, the immune checkpoints are PD-1, PD-L1, or CTLA-4. For example, gene editing techniques are used to knock out endogenous FAS, B2M, TCR and/or NKG2D ligands. For example, gene editing techniques are used to knock out endogenous FAS, B2M, TCR and/or NKG2D ligands. For example, CRISPR technology is used to knock out endogenous FAS, B2M, TCR and/or NKG2D ligands. For example, a polynucleotide fragment encoding NKG2D-CAR is inserted into the endogenous TCR or B2M or FAS gene so that the gene is interrupted.

The engineered cells of the present application constitutively or regulatory expressing a chimeric receptor (e.g., NKG2D-CAR) that recognizes a NKG2D ligand have one or any combination of the following uses: 1) for preparing universal CAR-T cells; 2) for resisting autologous or allogeneic immune cell killing and having anti-tumor effects; 3) for resisting autologous or allogeneic NK cell killing; 3) for resisting host immune rejection and anti-solid tumor effects; 4) for enhancing the survival and proliferation of another immune cell that is administered previously, simultaneously, or subsequently during in vitro and in vivo culture; 5) for enhancing anti-tumor effect in vitro and in vivo of another immune cell that is administered previously, simultaneously, or subsequently; 6) in the absence of NK cells, regulatory NKG2D-CAR-T cells can significantly kill NKG2DL-positive tumor cells; 7) in the presence of NK cells, regulatory NKG2D-CAR-T cells can also significantly kill tumor cells: 8) after co-incubation with tumor cells, regulatory NKG2D-CAR-T cells have a strong killing effect on NK cells, and the longer the co-incubation time, the stronger the killing effect; and can partially resist the killing effect of NK cells. Regardless of the presence or absence of NK cells, UCAR-T cells expressing ZNKG2D-CD123-synE, ZNKG2D-DAP10-CD123-synE, or ZBB-NKG2D-CD123-synE (e.g., knockout or low expression of endogenous TCR/B2M or TCR/B2M/FAS) can effectively inhibit the growth of leukemia xenografts.

The present application provides a combination of NKG2D-CAR-T cells and T cells expressing chimeric receptors (such as CARs, or recombinant TCRs) that recognize tumor antigens. Optionally, the endogenous TCR/B2M or TCR/B2M/FAS of the two combined cells is knocked out. The NKG2D-CAR-T cells provided in the present application can promote the survival and/or expansion of T cells expressing chimeric receptors (such as CARs, or recombinant TCRs) that recognize tumor antigens in the presence of autologous or allogeneic immune cells (T, NK and/or NKT cells).

The chimeric polypeptide provided herein is a receptor that regulates transcriptional activity in a target molecule-dependent manner. The chimeric polypeptide of the present application is a recombinant, non-naturally occurring receptor, comprising a binding domain, a receptor regulatory domain and an intracellular domain.

After the chimeric polypeptide binds to the target molecule, the chimeric polypeptide is triggered to be hydrolyzed, releasing the intracellular domain. The chimeric polypeptide can bind to a target molecule (e.g., a tumor antigen, a tissue-specific marker) displayed on the surface of a target cell, triggering the hydrolysis of the chimeric polypeptide to release the intracellular domain. Exemplarily, after the chimeric polypeptide binds to a tumor antigen on the surface of a tumor cell, it regulates a transcription factor that customizes the transcriptional program in the cell expressing the chimeric polypeptide. For example, after the chimeric polypeptide binds to a brain tissue marker, it regulates a transcription factor that customizes the transcriptional program in the cell expressing the chimeric polypeptide.

The chimeric polypeptide of the present application comprises, from N-terminus to C-terminus: (a) a binding domain capable of specifically binding to a target molecule, (b) a receptor regulatory domain comprising one or more cleavage sites, wherein the extracellular domain and the transmembrane domain of the receptor regulatory domain are not derived from Notch at the same time, and c) an intracellular domain; wherein the binding of the binding domain to the target molecule can induce the cleavage of the receptor regulatory domain and release the intracellular domain. The binding domain capable of specifically binding to the target molecule and the intracellular domain are heterologous to the Notch receptor polypeptide.

The receptor regulatory domain of the chimeric polypeptide comprises an extracellular domain and a transmembrane domain (also called a transmembrane region). The receptor regulatory domain comprises one or more ligand-induced proteolytic cleavage sites, wherein the cleavage sites are selected from I-CLiPs (intramembranously cleaving proteases) enzyme cleavage sites or sheddase protease cleavage sites. I-CLiPs are transmembrane cleaving proteases that can catalyze the hydrolysis of specific sites on the transmembrane domain of transmembrane proteins. The I-Clips may comprise a γ-secretase cleavage site. The γ-secretase cleavage site may comprise a γ-secretase cleavage site of a Gly-Val dipeptide sequence. The sheddase protease may be selected from: BACE1, ADAM8, ADAM9, ADAM10, ADAM12, ADAM17, MT1-MMP, or any combination thereof.

For example, the receptor regulatory domain comprises one or more ligand-induced proteolytic cleavage sites located in the transmembrane domain, and the cleavage sites are selected from the I-CLiPs enzyme cleavage sites.

The receptor regulatory domain comprises a transmembrane domain which may be a transmembrane domain of the single-pass transmembrane receptor, comprising at least one γ-secretase cleavage site. In one embodiment, the transmembrane domain includes, but is not limited to, the transmembrane domains of CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A and IFNAR2, wherein the transmembrane domain comprises at least one γ-secretase cleavage site. In one embodiment, the transmembrane domain includes, but is not limited to, the transmembrane domains of IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGR, ROBO1, SORCS3, SORCS1, SORL1, SDC1, SDC2, SPN, TYR, TYRP1, DCT, VASN, FLT1, CDH5, PKHD1, NECTIN1, PCDHGC3, NRG1, LRP1B, CDH2, NRG2, PTPRK, SCN2B, Nradd, and PTPRM. For example, the transmembrane domain comprises Notch1 transmembrane domain, Notch2 transmembrane domain, Notch3 transmembrane domain or Notch4 transmembrane domain from human or non-human animals (e.g., mouse, zebrafish, fruit fly, Xenopus or Gallus). For example, the transmembrane domain comprises an APLP1 transmembrane domain or an APLP2 transmembrane domain from human or non-human animals. For example, the carboxyl terminus of the transmembrane domain comprises a stop transfer sequence (STS). The STS connects the intracellular domain of the chimeric polypeptide and prevents it from entering the endoplasmic reticulum lumen.

The extracellular domain of the chimeric polypeptide provided herein comprises the extracellular full-length of Jagged2, EphrinB2, APLP1, APLP2, APP, CD44, CSF1R, CXCL16, CX3CL1, Delta1, E-cadherin, EphB2, EphrinB1, Growth hormone receptor, HLA-A2, IFNaR2, IL1R2, L1, LRP, LRP2, LRP6, N-cadherin, Nectin1α, NRADD, p75-NTR, Pcdh α4, Pcdh γ-C3, PTPκ, PTP-LAR, SorCS1b, SorLA, Sortilin, ApoER2, PKHD1, ErbB4, IFNaR2, VEGF-R1, or VLDLR, or a fragment of the extracellular domain of any of the above proteins or a truncation or truncated structure thereof, or a variant of the extracellular domain of any of the above proteins.

The chimeric polypeptides synE, synE-del1, synE-del2, synE-del3, and synE-del23, which comprise the extracellular domain of the receptor regulatory domain comprise the above-mentioned EphrinB2EC, EphrinB2EC-del1, EphrinB2EC-del2, EphrinB2EC-del3 and EphrinB2EC-del23, respectively, can trigger the cleavage of the chimeric polypeptide and release the intracellular domain after specifically binding to the target molecule. The leakage of induced expression of synE-del23 or synE-del23 is significantly reduced.

The present application constructs modified bodies of the EphrinB2 extracellular domain, EphrinB2-(EGF-like domain) n, wherein n=1 (SEQ ID No: 7), n=2 (SEQ ID No: 8), n=3 (SEQ ID No: 9), and n=4 (SEQ ID No: 10). The above-mentioned modified bodies of EphrinB2 respectively form corresponding receptor regulatory domains synE-EGF1, synE-EGF2, synE-EGF3, and synE-EGF4 with the Notch1 transmembrane domain (SEQ ID No: 15). The chimeric polypeptides comprising synE-EGF1, synE-EGF2, synE-EGF3 and synE-EGF4 can all trigger the cleavage of the chimeric polypeptides and release the intracellular domain after specifically binding to the target molecule; compared with before the modification, the background activation level of the chimeric polypeptides comprising the modified bodies is reduced in the absence of antigen stimulation. The EphrinB2 truncation comprises any of the above modifications. The truncation of the EphrinB2EC-del1, EphrinB2EC-del2, EphrinB2EC-del3 or EphrinB2EC-del23 comprises any of the above modifications.

Fragments, variants or truncated structures (also referred to as truncated forms or truncations) of the extracellular domain of EphrinB2 may sometimes be used interchangeably, and is a sequence having at least about 60%, 70%, 80%, 85%, 90%, 95%, 96%, 97%, 98%, 99% identity with the sequence of the extracellular domain of EphrinB2. The amino acid sequence of the truncated structure may be represented by SEQ ID NO: 3, 4, 5 or 6.

In one embodiment, the fragment, variant or truncated structure of the extracellular domain of EphrinB2 is a structure in which the potential site of the extracellular domain of EphrinB2 to be cleaved by ADAM10 is removed or mutated.

In one embodiment, the extracellular domain comprises the full-length EphrinB2 extracellular domain of human or non-human animals (e.g., white-cheeked gibbon, bonobo, Sumatran orangutan, chimpanzee, gorilla, rabbit, Peruvian night monkey, marmoset, Propithecus coquereli, small-eared macaque, mouse, rat, cow, African clawed frog), or a truncated or mutant or modified body thereof. For example, the extracellular domain comprises a fragment having at least 60%, 65%, 70%, 75%, 80%, 85%, 90%, 95%, 96%, 97%, 98%, 99% or 100% homology or identity with the amino acid sequence represented by SEQ ID NO: 1, 3, 4, 5, 6, 7, 8, 9, 10 or 65, and/or may optionally comprise at most 1, 2, 3, 4, 5 or more amino acid residues being replaced by different amino acid residues.

The receptor regulatory domain comprised in the chimeric polypeptide provided herein has an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity or homology with the amino acid sequences represented by SEQ ID NO: 60, 61, 62, 63, 64, 66 or 67.

The receptor regulatory domain of the chimeric polypeptide is short, which is conducive to viral packaging and expression and reduces the difficulty of industrial production and preparation. The full length of the coding sequence of the chimeric polypeptide synE or truncation or modified body is shorter than the full length of the synNotch coding sequence. The vector finally prepared comprising the intact chimeric polypeptide has high cell transduction efficiency and reduces the difficulty of industrial production and preparation.

The chimeric polypeptide synE or mutant or truncation or modified body thereof, and the gene that triggers regulation by binding thereto are not placed in the same vector, that is, a dual vector system. The chimeric polypeptide synE or mutant or truncation or modified body thereof is placed in the same vector with the gene that triggers regulation by binding thereto, that is, a single vector system. The single-vector system infection efficiency of synE or mutants or truncations or modified bodies thereof is higher than that of synNotch, thus reducing the difficulty of industrial production and preparation.

The leakage expression level under transcriptional regulation induced by chimeric polypeptide synE or mutant or truncation or modified body thereof is significantly reduced. The chimeric polypeptide synE or mutant or truncation or modified body thereof has both rigorous induction expression and strong induction ability. For example, the chimeric polypeptide synE or mutant or truncation or modified body thereof can better distinguish different expression levels of the same target molecule, and is suitable for identifying some target molecules that are lowly expressed in normal tissues and highly expressed in tumor tissues, and is not likely to cause off-target effects in normal tissues. For example, the chimeric polypeptide synE or mutant or truncation or modified body thereof regulates gene expression more strictly and have weaker sensitivity to low-expressed target molecules. When targeting some target molecules that are highly expressed in tumors and lowly expressed in normal tissues, synE or mutant or truncation or modified body thereof will have stronger selectivity for tumor tissues and higher safety.

The intracellular domain of the chimeric polypeptide of the present application comprises a protein fragment selected from any one of the following proteins or any combination thereof: transcription factors (comprising transcription activator proteins, transcription repressor proteins), transcription co-activators, transcription co-repressors, DNA binding polypeptides, RNA binding polypeptides, translation regulatory polypeptides, hormones, cytokines, toxins, antibodies, chromatin regulators, suicide proteins, organelle-specific polypeptides (such as nuclear pore regulators, mitochondrial regulators, endoplasmic reticulum regulators, etc.), pro-apoptotic polypeptides, anti-apoptotic polypeptides, other polypeptides that promote cell death by other mechanisms, pro-proliferation polypeptides, anti-proliferation polypeptides, immune co-stimulatory polypeptides, site-specific nucleases, recombinases, inhibitory immune receptors, activating immune receptors, variants of Cas9 and RNA targeting nucleases, DNA recognition polypeptides, signaling polypeptides, receptor tyrosine kinases, non-receptor tyrosine kinases, and polypeptides that promote differentiation.

The intracellular domain of the chimeric polypeptide of the present application comprises a transcriptional activator protein, which promotes or inhibits the transcription of a promoter-driven DNA sequence. For example, transcription factors directly regulate cell differentiation. For example, transcription factors indirectly regulate cell differentiation by regulating the expression of second transcription factors. For example, a transcription factor is a transcription activator proteins or a transcription repressor proteins. For example, transcription factors are transcriptional repressor proteins. For example, transcription factors are transcriptional activator proteins. For example, transcription factors also comprise nuclear localization signals. For example, the transcription factor is selected from the group consisting of: Gal4-VP16, Gal4-VP64, tetR-VP64, ZFHD1-VP64, Gal4-KRAB and HAP 1-VP16. For example, the transcription factor is Gal4. For example, the transcription factor is Gal4-VP64.

The antibodies induced by the intracellular domain of the chimeric polypeptide can be therapeutic antibodies for treating diseases (comprising immune diseases and tumors).

The intracellular domain of the chimeric polypeptide may comprise a protein fragment selected from any one of the following proteins or any combination thereof: a transcriptional activator protein, a transcriptional repressor protein, a site-specific nuclease, a recombinase, an inhibitory immune receptor, an activating immune receptor. For example, the transcriptional activator protein comprises GLA4, GLA4-VP64 or any fragment thereof. For example, the transcriptional activator protein comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity or homology with SEQ ID NO:28. For example, the intracellular domain of the chimeric polypeptide is a Cas9 polypeptide. For example, the intracellular domain of the chimeric polypeptide is a recombinase. For example, the intracellular domain of the chimeric polypeptide is an inhibitory immunoreceptor. For example, the intracellular domain of the chimeric polypeptide is an activating immune receptor.

The chimeric polypeptide may comprise an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity or homology with the amino acid sequence represented by SEQ ID NO: 44, 45, 46, 47, 48 or 49.

The chimeric polypeptide may comprise an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or homology with any one of the amino acid sequences obtained by sequentially linking an extracellular domain represented by SEQ ID NO: 1, 3, 4, 5, 6, 7, 8, 9, 10 or 65, a transmembrane domain represented by SEQ ID NO: 11, 12, 13, 14, 15, 16, 17, 18, 19, 20, 21 or 22, and an intracellular domain represented by SEQ ID NO: 28. The chimeric polypeptides provided herein comprise a binding domain that specifically binds to a target molecule. The binding domain of the chimeric polypeptide can specifically bind to one or more target molecules. For example, a chimeric polypeptide comprises a linker inserted between the binding domain and the receptor structural regulatory domain. For example, the binding domain comprises an antibody, an antigen, a ligand, a receptor, a target (e.g., a tag FLAG), an Fc receptor, an extracellular matrix component, a cell adhesion molecule, a non-antibody molecular scaffold, or any combination thereof. For example, the binding domain of the chimeric polypeptide comprises an antigen binding domain. For example, antigen binding domains comprises antibody-based recognition scaffolds. For example, an antigen binding domain comprises an antibody. For example, the antigen binding domain comprises an antibody that specifically binds a tumor antigen, a disease-associated antigen, or an extracellular matrix component. For example, the antigen binding domain comprises an antibody that specifically binds a cell surface antigen, a soluble antigen, or an antigen immobilized on an insoluble substrate. For example, the antigen binding domain comprises a single-chain antibody Fv (scFv). For example, an antigen binding domain comprises an antibody that can specifically bind to multiple antigens. For example, the antigen binding domain comprises a nanobody, a single domain antibody, a diabody, a triabody, a minibody, or any combination thereof. For example, the antigen binding domain is a non-antibody based recognition scaffold, such as an avimer, DARPin, ad nectin, avimer, affibody, anticalin, or affilin. For example, the antibody is a single domain antibody, a single chain antibody, a diabody, a triabody, a minibody, a F(ab′)2 fragment, a F (ab) v fragment, a scFv, a single domain antibody (sdAb), and functional fragments thereof, or any combination thereof.

The binding domain of the chimeric polypeptide can comprise an antigen, such as an endogenous antigen or an exogenous antigen. For example, a binding domain comprises a ligand for a receptor. For example, a binding domain comprises a receptor. For example, a binding domain comprises a cell adhesion molecule (e.g., the entire or a portion of an extracellular domain of a cell adhesion molecule). For example, a binding domain comprises a partial domain of a polymerisation domain.

The binding domain of the chimeric polypeptide specifically binds to the antigen referred to in the term “tumor antigen” or “pathogen antigen” in the present application. For example, the binding domain comprises an antibody that specifically binds to CD38, GPRC5D, Mesothelin, FAP, Claudin18.2, CLL1, CD19, GPC3, WT1, HER2, EGFR, EGFRVIII, BCMA, CD123, MOG, B7H3, NKG2A, FcRH5, or any combination thereof. For example, the binding domain comprises an VH or VL or scFV of an antibody, which specifically binds to Mesothelin, FAP, Claudin18.2, CLL1, CD19, GPC3, WT1, HER2, EGFR, EGFRVIII, BCMA, CD123, MOG, B7H3, NKG2A, FcRH5 or any combination thereof. For example, the binding domain comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity or homology with the amino acid sequence represented by SEQ ID NO:43, 68, 69, 70, 71, 72, 73, 74, 75, 76, 77, 78, 79, 80, 81, 82, or 83. For example, the binding domain comprises: an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity or homology with the amino acid sequence represented by SEQ ID NOs: 116 and 129, SEQ ID NOs: 117 and 130, SEQ ID NOs: 118 and 131, SEQ ID NOs: 119 and 132, SEQ ID NOs: 120 and 133, SEQ ID NOs: 121 and 134, SEQ ID NOs: 122 and 135, SEQ ID NOs: 123 and 136, SEQ ID NOs: 124 and 137, SEQ ID NOs: 125 and 138, SEQ ID NOs: 126 and 139, SEQ ID NOs: 127 and 140, and/or, SEQ ID NOs: 128 and 141.

The present application provides a chimeric polypeptide that specifically binds to Mesothelin, FAP, Claudin18.2, CLL1, CD19, GPC3, WT1, HER2, EGFR, EGFRVIII, BCMA, CD123, MOG, B7H3, NKG2A or FcRH5. For example, the chimeric polypeptide comprises: an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or homology with any one of the amino acid sequences obtained by sequentially connecting a binding domain represented by SEQ ID NO: 43, 68, 69, 70, 71, 72, 73, 74, 75, 76, 77, 78, 79, 80, 81, 82, or 83, an extracellular domain represented by SEQ ID NO: 1, 3, 4, 5, 6, 7, 8, 9, 10, or 65, a transmembrane domain represented by SEQ ID NO: 11, 12, 13, 14, 15, 16, 17, 18, 19, 20, 21, or 22, and an intracellular domain represented by SEQ ID NO:28.

Jurkat responding cells or human T cells comprising chimeric polypeptides GPC3-synE, GPC3-synE-del1, GPC3-synE-del2, GPC3-synE-del3, GPC3-synE-del23, or GPC3-synE-EGFn (n=1, 2, 3, 4) that recognize the tumor antigen GPC3 have the following characteristics: after co-incubation with GPC3-positive tumor cells SK-Hep1-GPC3, HuH7, or PLC/PRF/5, gene expression can be induced; after co-incubation with GPC3-negative tumor cells SK-Hep1, gene expression cannot be induced.

Jurkat responding cells or human T cells comprising the chimeric polypeptide MOG-synE, MOG-synE-del1, MOG-synE-del2, MOG-synE-del3 or MOG-synE-del23 that recognizes the tumor antigen MOG have the following characteristics: after co-incubation with MOG-positive cells K562-mMOG, K562-hMOG (also known as K562-huMOG), 293T-mMOG, or 293T-Hmog (also known as 293T-huMOG), gene expression can be induced; after co-incubation with MOG-negative cells K562 and 293T, gene expression cannot be induced.

Jurkat responding cells or human T cells containing chimeric polypeptides CD123-synE, CD123-synE-del1, CD123-synE-del2, CD123-synE-del3 or CD123-synE-del23 that recognize the tumor antigen CD123 have the following characteristics: gene expression can be induced after co-incubation with CD123-positive cells Molm13, MV-4-11 or THP-1; after co-incubation with CD123-negative cells, gene expression cannot be induced.

In one embodiment, the chimeric polypeptide comprises is a nucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity or homology with the nucleotide sequence represented by SEQ ID NO:55, 56, 57, 58, or 59, or the amino acid sequence encoded thereby.

The target molecule that binds to the chimeric polypeptide of the present application is also called a ligand or a target antigen.

The target molecule bound by the chimeric polypeptide may be membrane bound. The target molecule may be present on the surface of a cell. The target molecule can be immobilized on an insoluble substrate (e.g., polyethylene, polystyrene, polyvinyl pyrrolidone, polycarbonate, nitrocellulose, etc.). The target molecule may be soluble. The target molecule may be present in the extracellular environment (e.g., the extracellular matrix). The target molecule may be present in an artificial matrix. The target molecule may be present in a non-cellular environment. The target molecule is presented on an insoluble support which can take a variety of forms, such as plates, tissue culture dishes, columns, etc. The target molecule may be present in the extracellular matrix (ECM) (e.g., the antigen is a component of the ECM). The target molecule may be present in an artificial matrix. The target molecule may be present in a non-cellular environment. Target molecules comprise polypeptides, nucleic acids, glycoproteins, small molecules, carbohydrates, lipids, glycolipids, lipoproteins, and lipopolysaccharides. For example, the target molecule is selected from the group consisting of: a cluster of differentiation markers, a cell surface receptor, an adhesion protein, an integrin, a mucin, a lectin, and a tumor antigen.

The target molecule bound by the chimeric polypeptide may be a cluster of differentiation (CD) marker. For example, the CD marker is selected from the group consisting of: CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140(PDGFR4), CD152, CD154, CD158, CD178, CD181 (CXCR1), CD182 (CXCR2), CD183(CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, alkaline phosphatase, placental-like 2 (ALPPL2), B cell maturation antigen (BCMA), blue fluorescent protein (BFP), green fluorescent protein (GFP), enhanced green fluorescent protein (EGFP), signal regulatory protein alpha (SIRPa).

The target molecule bound by the chimeric polypeptide may be an antigen. For example, target molecules comprise tumor antigens and/or pathogen antigens. For example, the tumor antigen is selected from the group consisting of: GPRC5D, Mesothelin, FAP, Claudin18.2, CLL1, CD19, GPC3, WT1, EGFR, BCMA, B7H3, CD7, NKG2D-Ligand, CD19, B7H3, ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRVIII, ELF2M, EpCAM, EphA2, FLT3, GD2, GD3, GM3, HER2 (ERBB2), IGLL1, IL 11Ra, IL13Ra2, CD 117, MUC1, NCAM, PAP, PDGFR-b, PRSS21, PSCA, PSMA, ROR1, SIRPa, SSEA-4, TAG72, TEM1/CD248, TEM7R, TSHR, VEGFR2, ALPI, FcRH5, cMet and Axl.

In one embodiment, the chimeric polypeptide and the molecules expressed under its transcriptional regulation their expression recognize different tumor antigens, respectively.

In the treatment of tumors with antigenic heterogeneity, the proportion of cells positive for the first target molecule (exemplarily, EGFRvIII) (i.e., the positive rate) is low, and the positive rate of the second target molecule (exemplarily, IL13Ra2) is high; such tumor cells will escape the treatment targeted at the first target molecule, thereby reducing the efficacy; due to the widespread expression of the second target molecule, the treatment targeted at the second target molecule may produce off-target toxicity. For example, a chimeric polypeptide that binds to a first target molecule is constructed to trigger transcriptional regulation for the expression of an exogenous receptor recognizing a second target molecule, thereby achieving specific and extensive killing of tumor cells that express the second target molecule and partially express the first target molecule. For example, the killing effect of the CAR targeting the second target molecule depends on the triggered transcriptional regulatory activity of the binding of the chimeric polypeptide to the first target molecule, that is, the killing effect requires the presence of both the first target molecule and the second target molecule in the environment. The chimeric polypeptide synE or mutant or truncation or modified body thereof that binds to the first target molecule is used to trigger and regulate the expression of an exogenous receptor (such as CAR) targeting the second target molecule. Compared with synNotch, the anti-tumor effect of CAR-T cells transcriptionally regulated by the chimeric polypeptide on tumors with heterogeneity in expression of the first target molecule can be significantly improved.

In the treatment of tumors with target molecule heterogeneity, the first target molecule (exemplary, Mesothelin) is highly expressed but also expressed in normal tissues, and the second target molecule (exemplary, Claudin18.2) is highly expressed but also expressed in other normal tissues; using a treatment targeting either the first target molecule or the second target molecule alone will lead to off-target toxicity. For example, using a chimeric polypeptide synE or mutant or truncation or modified body thereof, synJagged2EC, synE-APLP2™ that specifically binds to the first target molecule to trigger the transcriptional regulation of the expression of an exogenous receptor targeting the second target molecule, can significantly improve the anti-tumor effect of CAR-T cells whose transcriptional regulation is triggered by the binding of chimeric polypeptides on tumors with heterogeneous expression of both the first and second target molecules compared to synNotch. For example, using a chimeric polypeptide synE or mutant or truncation or modified body thereof, synJagged2EC, synE-APLP2™ that specifically binds to the second target molecule to the trigger regulation of the expression of an exogenous receptor targeting the first target molecule, can significantly improve the anti-tumor effect of CAR-T cells whose transcriptional regulation is triggered by the binding of chimeric polypeptides on tumors with heterogeneous expression of both the first and second target molecules compared to synNotch.

In one embodiment, cells expressing CAR targeting a second target molecule may kill each other during culture, thereby affecting the activity and yield of the cells. For example, during the in vitro culture process of cells expressing a chimeric polypeptide targeting a first target molecule (e.g., a tumor antigen), since the cells do not come into contact with tumor cells expressing the tumor antigen, the chimeric polypeptide does not trigger transcriptional regulation of the expression of a chimeric receptor (e.g., CAR, recombinant TCR) targeting a second target molecule contained in the cells, thereby reducing the mutual killing phenomenon of the cells during in vitro and in vivo culture, thereby increasing the survival and/or proliferation of the cells during in vitro culture. For example, compared with constitutively expressed NKG2D-CAR-T cells, immune cells containing NKG2D-CAR regulated by a chimeric polypeptide that recognizes CD123 have enhanced survival ability in vitro and in vivo, and enhanced anti-tumor effects.

The immune cells expressing the regulatory NKG2D-CAR regulated by the chimeric polypeptide can resist NK cell killing and increase the persistence and/or transplantation survival rate of the immune cells expressing the regulatory NKG2D-CAR in the presence of host immune cells. For example, it can be used to prepare universal CAR-T cells. For example, NKG2D-CAR-T cells whose expression is regulated by a chimeric polypeptide are combined with T cells expressing a chimeric receptor (such as CAR) that recognizes a tumor antigen.

A chimeric polypeptide that specifically binds to a first target molecule is used to trigger transcriptional regulation of the expression of a CAR that targets a second target molecule. After the cell specifically binds to the first target molecule on a tumor cell, transcriptional regulation is triggered to express an exogenous receptor in the cell that targets the second target molecule, thereby killing the tumor cells that express the second target molecule, and/or attacking host immune cells that express the second target molecule to increase the survival and proliferation of the cells, thereby further improving the anti-tumor activity. For example, the second target molecule comprises NK cell markers selected from: NKG2 receptor family, such as NKG2A, NKG2D, NKG2D ligand, NKG2C, NKG2D-ligand, etc.; killer immunoglobulin-like receptor (KIR) family, such as KIR2DL1, KIR2DL2/3, KIR2DL4, KIR2DL5, KIR3DL1, 15KIR3DL2, KIR2DS1, KIR2DS2/S3, KIR2DS4, KIR2DS5, KIR3DS1, etc.; natural cytotoxicity receptors (NCR), such as NKP30, NKP44, NKP46, NKp80, etc.; and other NK cell-specifically expressed antigens, such as CD159a, CD159c, CD94, CD158, CD56, LIR/ILT2, CD244, CD226, CD2, CD16, CD161, TIGIT, CS1. For example, a chimeric polypeptide that specifically binds to a first target molecule of a leukemia cell (e.g., CLL1, CD123) is used to trigger transcriptional regulation of the expression of an exogenous receptor that recognizes the NKG2D ligand, thereby killing host NK cells and resisting host immune rejection. For example, the second target molecule is a NKG2D ligand. In one embodiment, an immune cell comprising a chimeric polypeptide that recognizes a tumor antigen and regulate the expression of a CAR that recognizes an NKG2D ligand can kill host NK cells or resist immune rejection of host NK cells. For example, an immune cell comprising a chimeric polypeptide that recognizes CD123 or CLL1 and regulates the expression of a CAR that recognizes an NKG2D ligand can kill host NK cells or resist immune rejection of host NK cells, thereby improving the survival and proliferation of the immune cells in vivo and in vitro, and enhancing the anti-tumor effect. For example, an immune cell comprising a chimeric polypeptide that recognizes a tumor antigen of a solid tumor and regulates the expression of a CAR that recognizes an NKG2D ligand can kill host NK cells or resist immune rejection of host NK cells. For example, an immune cell comprising a chimeric polypeptide that recognizes a tumor antigen of a solid tumor and regulates the expression of a CAR that recognizes an NKG2D ligand can kill host NK cells or resist immune rejection of host NK cells, thereby improving the survival and proliferation of the immune cells in vivo and in vitro and enhancing the anti-tumor effect. For example, the solid tumor cells express a NKG2D-ligand.

The chimeric polypeptide binding triggered regulatory genes provided in the present application is operatively linked to a transcription control element, wherein the transcription control element is activated or inhibited by the intracellular domain of the chimeric polypeptide. For example, the expression of regulated gene is regulated by a promoter regulated by GAL-4, tetR, ZFHD1, HNF1A or HAP1. For example, the regulated gene expression product is selected from: non-coding RNA, cytokine, cytotoxin, chemokine, immunomodulator, pro-apoptotic factor, anti-apoptotic factor, hormone, differentiation factor, dedifferentiation factor, recombinant TCR receptor, CAR, reporter gene, or any combination thereof.

The chimeric polypeptide binding triggered regulatory genes including, but not limited to, chemokines, chemokine receptors, cytokines, cytokine receptors, differentiation factors, growth factors, growth factor receptors, hormones, metabolic enzymes, proliferation inducers, receptors, small molecule second messenger synthetases, CARs, recombinant TCR receptors, T cell receptors, second chimeric polypeptides, transcription activators, transcription repressors, transcription activators, transcription repressors, translation regulators, antibody molecules, translation activators, translation repressors, activating immune receptors, apoptosis inhibitors, apoptosis inducers, immune activators, immune suppressors, and inhibitory immune receptors. For example, the nucleic acid sequence encoding the regulatory gene, or any combination thereof, is operatively linked to a transcriptional control element, which is activated or repressed by the intracellular domain of the chimeric polypeptide. For example, the transcriptional control element comprises UAS (SEQ ID NO: 29). For example, the intracellular domain of the chimeric polypeptide comprises GAL4-VP64 (SEQ ID NO: 28); the nucleic acid sequence of the chimeric polypeptide binding triggered regulatory genes is operatively linked to a UAS. For example, the intracellular domain of the chimeric polypeptide comprises GAL4-VP64; the nucleic acid sequence of the chimeric polypeptide binding triggered regulatory gene is operatively linked to a UAS-CMV promoter (SEQ ID NO: 30).

For example, after the chimeric polypeptide in the cell binds to the target molecule, it triggers the receptor to hydrolyze, causing the receptor to cleave and release the intracellular domain, thereby inducing the cell to express cytokines or chemokines comprising: interferons (α-interferon, β-interferon, and γ-interferon), interleukins (IL-1, IL-1a, IL-2, IL-3, IL-4, IL-5, IL-6, IL-7, IL-8, IL-9, IL-10, IL-11, IL-12; IL-13, IL-14, IL-15, IL-16, IL-17, IL-17A, IL-18, IL-19, IL-20, and IL-24), tumor necrosis factor (TNF-α), transforming growth factor-β, TRAIL, MIP-1, MIP-1β, MCP-1, RANTES, IP10, CCL2, CCL3, CCL5, CCL17, CCL19, CCL21, CCR7, CXCL9, CXCL10, CXCL11, CXCL16, and MCSF. For example, the cytokines comprise IL-2, IL-7, IL-9, IL-12, IL-15, IL-18, CCL21, or any combination thereof.

Cells (e.g., T, NK and/or NKT cells) can be transduced with a viral vector encoding the chimeric polypeptide. The viral vector can be a lentiviral vector or a retroviral vector. The viral vector may be a pRRLSIN vector. The transduced cells can stably express the chimeric polypeptide. When the chimeric polypeptide is localized on the cell membrane, it can be hydrolyzed in the transmembrane domain after recognizing the target molecule, and release the intracellular transcription regulatory domain GAL4-VP64, which is transferred into the cell nucleus and can play a transcriptional activation role on the promoter comprising the UAS sequence. The regulated gene is connected to the downstream of the UAS promoter and transferred into the cell expressing the chimeric polypeptide, so that the target-dependent specific expression of the target gene regulated by the chimeric polypeptide can be achieved.

In one embodiment, after binding to the first target molecule, the chimeric polypeptide in the cell triggers the receptor to undergo hydrolysis, causing the receptor to cleave, releasing the intracellular domain, and inducing the cell to express a CAR or recombinant TCR that recognizes the second target molecule. The nucleic acid sequence of the CAR or recombinant TCR is operatively linked to a transcriptional control element, which is activated or inhibited by the intracellular domain of the chimeric polypeptide. For example, the CAR or recombinant TCR specifically recognizes: tumor antigens, cancer (or tumor) cell-associated antigens, hematological tumor antigens (or liquid tumor antigens), solid tumor antigens, cell surface antigens, and intracellular antigens, etc.

In one embodiment, the first target molecule and the second target molecule are different and are respectively selected from the group consisting of: 1) hematological tumor antigens (or liquid tumor antigens): CD19 (expressed on B cells), CD20 (expressed on B cells), CD22 (expressed on B cells), CD30 (expressed on B cells), CD33 (expressed on bone marrow cells), CD70 (expressed on B cells/T cells), CD123 (expressed on bone marrow cells), K (expressed on B cells), Lewis Y (expressed in bone marrow cells), NKG2D ligand (expressed in bone marrow cells), ROR1 (expressed in B cells), SLAMF7/CS1 (expressed in myeloma cells, natural killer cells, T cells and most B cells), CD138 (expressed in malignant plasma cells in multiple myeloma), CD56 (expressed in myeloma cells, neurons, natural killer cells, T cells and trabecular osteoblasts), CD38 (expressed in B cells/T cells) and CD160 (expressed in NK cells/T cells); 2) solid tumor antigens: B7H3 (expressed in malignant tumor and glioma), CAIX (expressed in kidney), CD44v6/v7 (expressed in cervix), CD171 (expressed in neuroblastoma), CEA (expressed in colon), EGFRvIII (expressed in gliomas), EGP2 (expressed in cancer), EGP40 (expressed in colon), EphA2 (expressed in glioma and lung), ErbB2 (HER2) (expressed in breast, lung, prostate and glioma), ErbB receptor family (expressed in breast, lung, prostate and glioma), ErbB3/4 (expressed in breast and ovary), HLA-A1/MAGE1 (expressed in melanoma), HLA-A2/NY-ESO-1 (expressed in malignant tumor and melanoma), FR-a (expressed in ovary), FAR (expressed in rhabdomyosarcoma), GD2 (expressed in neuroblastoma, malignant tumor and melanoma), GD3 (expressed in melanoma and lung cancer), HMW-MAA (expressed in melanoma), IL11Ra (expressed in osteosarcoma), IL13Ra2 (expressed in glioma), Lewis Y (expressed in breast/ovary/pancreas), mesothelin (expressed in mesothelioma, breast and pancreas), Muc1 (expressed in ovary, breast and prostate), NCAM (expressed in neuroblastoma and colorectal), NKG2D ligand (expressed in ovary and malignant tumors), PSCA (expressed in prostate and pancreas), PSMA (expressed in prostate), TAG72 (expressed in colon), VEGFR-2 (expressed in tumor vasculature), Axl (expressed in lung cancer), Met (expressed in lung cancer), α5β3 (expressed in tumor vasculature), α5β1 (expressed in tumor vasculature), TRAIL-R1/TRAIL-R2 (expressed in solid tumor (colon, lung and pancreas) and hematological malignancy), RANKL (expressed in prostate cancer and bone metastasis), tenascin (expressed in glioma, epithelial tumor (breast and prostate), EpCAM (expressed in epithelial tumors (breast, colon and lung)), CEA (expressed in epithelial tumor (breast, colon and lung)), gpA33 (expressed in colorectal cancer), mucin (expressed in epithelial tumor (breast, colon, lung and ovary)), TAG-72 (expressed in epithelial tumor (breast, colon and lung)), EphA3 (expressed in lung, kidney, melanoma, glioma, hematological malignancy), and IGF1R (expressed in lung, breast, head and neck, prostate, thyroid and glioma). Examples of surface and intracellular antigens comprise, for example, Her2 (ERBB2), MAGE-A1 (MAGEA1), MART-1 (MLANA), NY-ESO (CTAG1), WT1, MUC17, MOG, and MUC13. In one embodiment, the first and second target molecules are selected from the group consisting of: BCMA, B7H6, CAIX, CD123, CD138, CD171, CD19, CD20, CD22, CD30, CD33, CD38, CD44, CEA, CS1, EGFRVIII, EGP2, EGP40, Erb family members (ERBB1, ERBB2, ERBB3, and ERBB4), FAP, fetal acetylcholine receptor (AChR), folate receptor alpha (FOLR1), folate receptor beta (FOLR2), GD2, GD3, GPC3, IL-13Ra2 (IL13RA2), kappa light chain (IGK), Lewis-Y, mesothelin (MSLN), mucin-1 (MUC1), mucin-16 (MUC16), NKG2D ligand, prostate-specific membrane antigen (PSMA), prostate stem cell antigen (PSCA), receptor tyrosine kinase-like orphan receptor 1 (ROR1), anaplastic lymphoma receptor tyrosine kinase (ALK), and MOG.

In one embodiment, the binding domain of the chimeric polypeptide specifically binds to a first target molecule, and the CAR specifically binds to a second target molecule different from the first target molecule, and the two are respectively selected from the group consisting of: Mesothelin, FAP, Claudin18.2, CLL1, CD19, GPC3, WT1, EGFR, EGFRVIII, BCMA, CD7, NKG2D-Ligand, MOG, CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRVIII, ELF2M, EpCAM, EphA2, Ephrin B2, FAP, FLT3, GD2, GD3, GM3, GPRC5D, HER2 (ERBB2/neu), IGLL1, IL 11Ra, IL13Ra2, CD 117, MUC1, NCAM, PAP, PDGFR-b, PRSS21, PSCA, PSMA, ROR1, SIRPa, SSEA-4, TAG72, TEM1/CD248, TEM7R, TSHR, VEGFR2, ALPI, citrullinated vimentin, cMet, and Axl.

Regulatory B7H3-CAR-T cells: T cells comprising a chimeric polypeptide binding MOG and B7H3-CAR; and the chimeric polypeptide binding MOG, MOG-synE, or mutant or truncation or modified body thereof, synJagged2EC, synE-APLP2™), synNTOCH, syn-CD8, can trigger transcriptional regulation of the expression of B7H3-CAR. Regulatory B7H3-CAR-T cells have one or more of the following characteristics: 1) after incubation with MOG-positive cells, they can effectively kill B7H3-positive glioma cells (such as U87, U251); 2) the cytokine IL2 is released only in the presence of MOG; 3) when co-incubated with cells with low or no expression of MOG, the killing effect on B7H3-positive glioma cells is weakened or there is no killing occurs. MOG-synE-del3 has a good regulatory ability on the anti-tumor effect initiated by CAR-T cells. The binding domain of the chimeric polypeptide can be a ligand/receptor that binds MOG; or an antibody that binds MOG: scFv, Fab, single domain antibody, or fully human antibody or fragment thereof.

Regulatory NKG2D-CAR-T cells: T cells comprising a chimeric polypeptide binding CD123 and NKG2D-CAR (binding to NKG2D ligand); and the chimeric polypeptide binding CD123, CD123-synE, or mutant, truncated or modified body thereof, synJagged2EC, synE-APLP2™), synNOTCH, syn-CD8, can trigger transcriptional regulation of the expression of NKG2D-CAR. Regulatory NKG2D-CAR-T cells have one or more of the following characteristics: 1) after incubation with CD123-positive cells, they can effectively kill NKG2D ligand-positive tumor cells (such as MV-4-11 and THP-1) or immune cells (such as NK, T, and NKT cells), but have weak killing effects on cells with low expression of NKG2D ligands (such as KG-1 and Molm13); 2) when co-incubated with cells with low or no CD123 expression, the killing effect on NKG2D ligand-positive cells is weakened or there is no killing effect (such as SK-Hep1 and K562). The killing ability of NKG2D-CAR-T cells regulated by CD123-synE or CD123-synE-del3 is NKG2D ligand-dependent, while high expression of CD123 alone (such as Molm13) will not cause NKG2D ligand-independent killing. Placing NKG2D-CAR under the regulation of CD123-synE or CD123-synE-del3 can improve the in vivo survival ability and anti-tumor effect of regulatory NKG2D-CAR-T cells. Regulatory NKG2D-CAR-T cells can recognize target cells through CD123-synE or CD123-synE-del3. The killing effect of NKG2D-CAR-T cells still requires the initiation of NKG2D ligands on the surface of target cells. There is no cross-influence between the downstream signals of CD123-synE or CD123-synE-del3 and the downstream signals of NKG2D-CAR.

In one embodiment, the transcription factor triggered by the chimeric polypeptide in response to the first target molecule drives the expression of the CAR in response to the second target molecule, so that the CAR is active and activates T cells only in the presence of both the first and second target molecules. For example, the first target molecule and the second target molecule are respectively ASGR1 and GPC3, EGFRVIII and IL13Ra2, EGFRVIII and B7H3, Mesothelin and Claudin18.2, Claudin18.2 and Mesothelin, FAP and Claudin18.2, CLL1 and NKG2D ligand, CD123 and NKG2D ligand, or MOG and B7H3. For example, the chimeric polypeptide and CAR respectively comprise: the sequence represented by SEQ ID NO: 46 and 50; or the sequence represented by SEQ ID NO: 47 and 50; or the sequence represented by SEQ ID NO: 48 and 51; or the sequence represented by SEQ ID NO: 48 and 52; or the sequence represented by SEQ ID NO: 48 and 53; or the sequence represented by SEQ ID NO: 48 and 54; or the sequence represented by SEQ ID NO: 49 and 51; or the sequence represented by SEQ ID NO: 49 and 52; or the sequence represented by SEQ ID NO: 49 and 53; or the sequence represented by SEQ ID NO: 49 and 54.

U87 and U251 cells express endogenous IL13Ra2 but not EGFRVIII. The killing of cells expressing at least two or more target molecules with different positive rates by T cells containing a chimeric polypeptide that regulates CAR expression is detected. For example, EGFRvIII-positive and EGFRvIII-negative cells were mixed in different proportions to simulate gliomas that were IL13Ra2-positive and only partially EGFRvIII-positive under natural conditions. IL13Ra2-CAR-T cells regulated by the chimeric polypeptide EGFRvIII-synE can significantly kill EGFRVIII antigen-heterogeneous tumors both in vivo and in vitro, and also have a killing effect on tumor cell populations with low EGFRvIII positivity rates.

The nucleic acid sequence encoding the CAR is operatively linked to a transcriptional control element that is regulated by the intracellular domain of the chimeric polypeptide. For example, the nucleic acid sequence encoding CAR is operatively linked to a UAS-CMV promoter, and the intracellular domain of the chimeric polypeptide comprises GLA4-VP64. For example, the extracellular antigen binding domain of CAR comprises an antibody that recognizes B7H3, Mesothelin, FAP, Claudin18.2, CLL1, CD19, GPC3, WT1, HER2, EGFR, EGFRVIII, BCMA, CD7, CD123, NKG2D ligands, or any combination thereof. For example, the extracellular antigen binding domain of CAR comprises an antibody scFV that recognizes B7H3, Mesothelin, FAP, Claudin18.2, CLL1, CD19, GPC3, WT1, HER2, EGFR, EGFRVIII, BCMA, CD7, CD123, NKG2D ligands, or any combination thereof. For example, the extracellular antigen binding domain of CAR comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity or homology with the amino acid sequence represented by SEQ ID NO: 68, 69, 70, 71, 72, 73, 74, 75, 76, 77, 78, 79, or 80. For example, an IL13Ra2-CAR, B7H3-CAR, Claudin18.2-CAR, Mesothelin-CAR or NKG2D-CAR whose expression is regulated by a chimeric polypeptide is operatively linked to a transcriptional control element that is regulated by the intracellular domain of the chimeric polypeptide. For example, IL13Ra2-CAR, B7H3-CAR, Claudin18.2-CAR, Mesothelin-CAR or NKG2D-CAR whose expression is regulated by a chimeric polypeptide is inserted downstream of the UAS-CMV promoter, and the intracellular domain of the chimeric polypeptide comprises Gal4-VP64. The “Z-CAR-T expressing X-chimeric polypeptide Y” or “X-chimeric polypeptide Y—Z-CAR-T” or “Z-CAR-X-chimeric polypeptide Y” described in the examples of the present application is used to describe a T cell (also referred to as a regulatory Z-CAR-T cell) comprising a chimeric polypeptide Y targeting antigen X that regulates the expression of a CAR that recognizes antigen Z, wherein the Z-CAR is inserted downstream of the UAS-CMV promoter, and the intracellular domain of the chimeric polypeptide Y comprises Gal4-VP64. In the above, the symbols X and Z are different tumor antigens, i.e., the chimeric polypeptide Y can target antigen X, and the CAR can recognize antigen Z. When the chimeric polypeptide Y and CAR are used in combination in T cells, the expression of the CAR is regulated by the intracellular domain of the chimeric polypeptide Y.

For example, gastric cancer highly expresses both Mesothelin and Claudin18.2. In order to prevent Claudin18.2-CAR-T cells from killing normal tissues expressing Claudin18.2, the killing effect of the Claudin18.2-CAR-T cells is limited to the tumor area. For example, The Claudin18.2-CAR-T cells expressing Mesothelin-synEphrinB2EC can significantly kill Mesothelin and Claudin18.2 double-positive gastric cancer cells.

The extracellular antigen binding domain of CAR can be directly connected to the transmembrane domain or connected through a hinge. For example, the hinge comprises a CD8 hinge, e.g., a sequence having 95-100% identity with SEQ ID NO:31. For example, a nucleic acid molecule encoding a CAR comprises a polynucleotide encoding a signal peptide upstream. For example, the signal peptide comprises a CD8 signal peptide, e.g., a sequence having 95-100% identity with SEQ ID NO:37.

The transmembrane domain of the CAR molecule of the present application may comprise a CD28 or CD8 transmembrane domain. For example, the CAR comprises a sequence having 95-100% identity with SEQ ID NO:32. For example, the CAR comprises a sequence having 95-100% identity with SEQ ID NO:33.

CAR comprises an intracellular signaling domain: a primary signaling domain and/or a co-stimulatory signaling domain. For example, the primary signaling domain comprises the CD35 intracellular domain, e.g., a sequence having 95-100% identity with SEQ ID NO:36. For example, the co-stimulatory signaling domain comprises CD28 and/or 4-1BB intracellular domains. For example, the CAR comprises a sequence having 95-100% identity with SEQ ID NO:34, 35, 38, 39 or 40.

The intracellular signaling domain of the CAR may comprise a human CD32 intracellular domain. The intracellular signaling domain of CAR may comprise a human CD32 intracellular domain and a CD28 intracellular domain. The intracellular signaling domain of CAR may comprise a human CD35 intracellular domain and a 4-1BB intracellular domain. The intracellular signaling domain of CAR may comprise a CD32 intracellular domain, a CD28 intracellular domain, and a 4-1BB intracellular domain.

Exemplarily, the CAR expressed under transcriptional regulation of the chimeric polypeptide in the present application comprises a sequence obtained by sequentially linking a sequence represented by SEQ ID NO: 68, 69, 70, 71, 72, 73, 74, 75, 76, 77, 78, 79, or 80 to a sequence represented by SEQ ID NO: 38, 39 or 40.

The chimeric polypeptide binding triggered regulatory genes including but not limited to those encoding: Trastuzumab (Trastuzumab, commercially available from Herceptin, Chugai Pharmaceutical Co., Ltd.); bevacizumab (Avastin, commercially available from Genentech, Inc.); infliximab (Remicade); rituximab (Rituxan, commercially available from Biogen Idec Inc.); and adalimumab (Humira). For example, the above therapeutic antibodies are inserted into downstream of the UAS-CMV promoter regulated by Gal4-VP64.

The chimeric polypeptide and/or chimeric receptor recognizing NKG2D ligand of the present application may also comprise one or more other domains, comprising: a signal peptide, an epitope tag, an affinity domain, a nuclear localization signal (NLS), and a polypeptide that generates a detectable signal. For example, a nucleic acid molecule encoding a chimeric polypeptide comprises a polynucleotide encoding a signal peptide upstream. For example, the signal peptide comprises a CD8 signal peptide, e.g., a sequence having 95-100% identity with SEQ ID NO:37.

The nucleotide sequence encoding the chimeric polypeptide of the present application is operatively linked to a transcription control element (e.g., a promoter, and an enhancer, etc.). For example, the transcriptional control element is inducible. For example, the transcriptional control element is constitutive. For example, promoters are functional in eukaryotic cells. For example, the promoter is a cell type specific promoter. For example, the promoter is a tissue-specific promoter.

The expression vector of the chimeric polypeptide and/or the chimeric receptor recognizing the NKG2D ligand of the present application can be a viral vector, for example, an adeno-associated virus (AAV) vector, an adenovirus vector, a lentivirus vector, and a retrovirus vector, etc. For example, the retroviral vectors (gamma-retroviruses or lentiviruses) are used to introduce nucleic acid molecules into cells. The non-viral vectors can also be used. Transduction can use any suitable viral vector or non-viral delivery system. In a specific embodiment of the present application, a pRRLSIN vector is constructed to express the chimeric polypeptide. In a specific embodiment of the present application, a pRRLSIN vector is constructed to express a transcriptional regulatory gene triggered by the binding of chimeric polypeptides, such as a transcriptional regulatory CAR triggered by the binding of the chimeric polypeptides. The chimeric polypeptides or CARs can be constructed with accessory molecules (e.g., cytokines) in a single polycistronic expression cassette, multiple expression cassettes in a single vector, or in multiple vectors. Examples of elements for generating polycistronic expression cassettes include, but are not limited to, various viral and non-viral internal ribosome entry sites (IRES, e.g., FGF-1 IRES, FGF-2 IRES, VEGF IRES, IGF-II IRES, NF-κB IRES, RUNX1 IRES, p53 IRES, Hepatitis A IRES, Hepatitis C IRES, Pestivirus IRES, Baculovirus-Free IRES, Picornavirus IRES, Poliovirus IRES, and Encephalomyocarditis Virus IRES) and cleavable linkers (e.g., 2A peptides, e.g., P2A, T2A, E2A, and F2A peptides).

Other viral vectors that can be used comprise, for example, adenovirus, lentivirus, adeno-associated viral, Bullpox virus, bovine papillomavirus or herpesvirus vectors, such as Epstein-Barr virus vectors.

Non-viral methods can also be used to genetic modifications of engineered cells. For example, nucleic acid molecules can be introduced into immune cells by lipofection, asialomucoid-polylysine conjugation, or microinjection under surgical conditions. Other non-viral gene transfer methods comprise in vitro transfection using transposons, liposomes, calcium phosphate, DEAE dextran, electroporation, and protoplast fusion. The nucleic acid molecules can also be transferred into a cell type that can be cultured in vitro (e.g., autologous or allogeneic primary cells or their progeny), and the cells (or their progeny) modified with the nucleic acid molecules are then injected into the target tissue of the subject or injected systemically.

The present application provides engineered cells genetically modified with the nucleic acids of the present application: engineered cells genetically modified with nucleic acids comprising encoding the chimeric polypeptides of the present application and/or chimeric receptors that recognize NKG2D ligands; or engineered cells genetically modified with nucleic acids comprising the chimeric polypeptides of the present application and nucleic acids comprising a gene whose transcriptional regulation is triggered by the binding of chimeric polypeptides (e.g., chimeric receptors that recognize NKG2D ligands). The present application provides a method for regulating the activity of a cell expressing the chimeric polypeptide of the present application. The methods generally comprise contacting an engineered cell with a target molecule, inducing cleavage of the chimeric polypeptide, thereby releasing the intracellular domain, and the release of the intracellular domain modulates the activity of the cell.

In one embodiment, the engineered cells are genetically modified to express the chimeric polypeptide of the present application, and are further genetically modified to express CAR. For example, the engineered cell is genetically modified with a nucleic acid comprising a nucleotide sequence encoding a CAR, and the intracellular domain of the chimeric polypeptide is a transcriptional activator protein, and the nucleotide sequence encoding the CAR is operatively linked to a transcriptional control element activated by the intracellular domain of the chimeric polypeptide. Many CAR polypeptides have been described in the art, and any of them are suitable for use in the present application.

In one embodiment, the engineered cell is an immune cell, a neuron, an epithelial cell, an endothelial cell, or a stem cell. Stem cells include human pluripotent stem cells (including human induced pluripotent stem cells (iPSCs) and human embryonic stem cells). For example, the engineered cells are immune cells. For example, the engineered cell is a primary cell. For example, the engineered cells are B cells, monocytes, natural killer cells, basophils, eosinophils, neutrophils, dendritic cells, macrophages, regulatory T cells, helper T cells, cytotoxic T cells, other T cells, or any combination thereof. The engineered cells can be cells of autologous origin or cells of allogeneic origin.

The present application provides a method for regulating the activity of an engineered cell expressing the chimeric polypeptide of the present application, comprising: contacting the engineered cell containing the chimeric polypeptide of the present application with a target molecule, wherein after binding of a binding domain of the chimeric polypeptide to the target molecule, a protease cleavage site of a receptor regulatory domain of the chimeric polypeptide is induced to be cleaved, wherein release of an intracellular domain regulates activity of the engineered cell. For example, the contacting is performed in vivo, ex vivo, or in vitro. For example, the target molecules are on the surface of target cells, immobilized on insoluble substrates, present in extracellular matrixes, present in artificial matrixes, or is soluble. For example, the release of the intracellular domains regulates proliferation of engineered cells. For example, the release of the intracellular domains regulates apoptosis of the engineered cells. For example, the release of the intracellular domains induces cell death by mechanisms other than apoptosis. For example, the release of the intracellular domains regulates gene expression in the engineered cells through transcriptional regulation, chromatin regulation, translation, transportation, or post-translational processing. For example, the release of the intracellular domains regulates differentiation of the engineered cells. For example, the release of the intracellular domains regulates migration of the engineered cells. For example, the release of the intracellular domain regulates expression and secretion of molecules from the engineered cells. For example, the release of the intracellular domains regulates adhesion of the engineered cells to target cells or to the extracellular matrixes. For example, the release of the intracellular domains induces re-expression of gene product in the engineered cells. For example, the release of the intracellular domains induces cessation of expression of the gene product in the engineered cells. For example, the gene products are transcriptional activators, transcriptional repressors, modified TCRs, chimeric antigen receptors, translation regulators, cytokines, hormones, chemokines, or antibodies. For example, the released transcription factors regulate the differentiation of the engineered cells, which are immune cells, stem cells, progenitor cells or precursor cells. For example, the method is used to treat tumors.

The present application provides a method for activating a T cell, comprising: contacting a T cell as described herein with a target molecule, wherein the T cell is genetically modified with one or more nucleic acids comprising nucleotide sequences encoding the following: i) a chimeric polypeptide involved in the present application, and ii) CAR; wherein the binding domain of the chimeric polypeptide comprises an antibody specific for a first target molecule, and wherein the contact results in release of an intracellular domain of the chimeric polypeptide (e.g., a transcriptional activator protein), expression of the CAR polypeptide in the T cell, wherein the CAR polypeptide activates the T cell after binding to a second target molecule. For example, the first and second target molecules are different tumor antigens; or the first target molecules are molecules with tissue specificity and the second target molecules are tumor antigens.

The present application provides a method for regulating activity of an engineered cell, comprising: contacting the engineered cell with an antigen immobilized on a surface of a target molecule, wherein the expression of the chimeric polypeptide of the present application by the engineered cell can lead to the release of an intracellular domain and regulate the activity of the engineered cell. For example, the intracellular domains are transcription factors that regulate cell differentiation.

The present application provides a method for locally regulating activity of an engineered cell, comprising: expressing a chimeric polypeptide comprising the present application in a cell; after the engineered cell contacts a target molecule, releasing an intracellular domain to regulate the activity of the engineered cell: expression of gene products of the cell, cell proliferation, apoptosis, non-apoptotic cell death, cell differentiation, cell dedifferentiation, cell migration, cell expression and secretion of a protein, and cell adhesion.

The present application provides a method for treating a tumor in a subject having the tumor, comprising: i) genetically modifying T, NK and/or NKT cells obtained from an individual using a vector comprising nucleic acid encoding a chimeric polypeptide of the present application, or the chimeric polypeptide and a transcriptional activated CAR triggered by binding of the chimeric polypeptide, wherein the chimeric polypeptide can specifically bind to an antigen expressed by a tumor cell in the subject; ii) introducing the genetically modified the T, NK and/or NKT cells into the subject, wherein the genetically modified the T, NK and/or NKT cells recognize and kill the tumor cell, thereby treating the tumor.

The present application provides a method for treating a tumor in a subject having the tumor, comprising: i) genetically modifying T, NK and/or NKT cells obtained from an individual using a vector comprising nucleic acid encoding a chimeric polypeptide of the present application, or the chimeric polypeptide and a transcriptional activated CAR triggered by binding of the chimeric polypeptide, wherein the chimeric polypeptide can specifically bind to a certain tissue-specific marker in the subject; ii) introducing the genetically modified the T, NK and/or NKT cells into the subject, wherein the genetically modified the T, NK and/or NKT cells recognize and kill the tumor cell, thereby treating the tumor.

The present application provides a method for inhibiting activity of a target cell in a subject, comprising: administering to the subject a therapeutically effective amount of an engineered cell expressing the chimeric polypeptide of the present application, wherein the engineered cell inhibits activity of the target cell in the subject and/or kills the target cell. For example, the target cells are tumor cells. For example, the target cells include, but are not limited to, acute myeloma leukemia cells, anaplastic lymphoma cells, astrocytoma cells, B-cell cancer cells, breast cancer cells, colon cancer cells, ependymoma cells, esophageal cancer cells, glioblastoma cells, glioma cells, leiomyosarcoma cells, liposarcoma cells, hepatoma cells, lung cancer cells, mantle cell lymphoma cells, melanoma cells, neuroblastoma cells, non-small cell lung cancer cells, oligodendroglioma cells, ovarian cancer cells, pancreatic cancer cells, peripheral T cell lymphoma cells, renal cancer cells, sarcoma cells, gastric cancer cells, hepatoma cells, mesothelioma cells and sarcoma cells. For example, the target cells express low levels of the target molecules. For example, the engineered cells further comprise transcriptional activated CARs, modified TCRs, exogenous cytokines and/or therapeutic monoclonal antibodies that are triggered by the binding of the chimeric polypeptides to target molecules. For example, the chimeric polypeptides specifically bind to first target molecules, and the CARs and/or modified TCRs specifically bind to second target molecules. For example, the first and second target molecules in the target cells are both tumor antigens, but there is expression heterogeneity in the tumors. For example, the first and second target molecules in the target cells are both tumor antigens, and the positive rate of the first target molecules in tumor tissues is lower than that of the second target molecules. For example, the first and second target molecules in the target cells are both tumor antigens, and the positive rate of the second target molecules in the tumor tissues is lower than that of the first target molecules. For example, the first target molecules are molecules having tissue specificity, and the second target molecules are the tumor antigens. For example, the first target molecules are not tumor antigens but has tissue-specific expression, and the second target molecules are the tumor antigens. For example, the positive rate of the first target molecules is lower than that of the second target molecules. For example, the method can improve the anti-tumor specificity of the engineered cells.

The present application provides a method for preventing, alleviating and/or treating a tumor, which comprises administering a cell composition and a pharmaceutical composition to a subject in need thereof. The cell composition and pharmaceutical composition provided in the present application have been described above, and the method for preventing, alleviating and/or treating a tumor provided in the present application comprises all technical solutions thereof.

The present application provides a kit for inducing and/or enhancing an immune response and/or treating and/or preventing a tumor or pathogen infection in a subject. For example, the kit comprises an effective amount of a composition and a pharmaceutical composition of the chimeric polypeptide and/or a chimeric receptor recognizing a NKG2D ligand of the present application. For example, the kit comprises a sterile container; such container may be in the form of a box, ampoule, bottle, vial, tube, bag, pouch, blister pack, or other suitable container known in the art. Such containers may be made of plastic, glass, laminated paper, metal foil, or other material suitable for containing medications. For example, the kit comprises molecules encoding a chimeric receptor that recognizes an NKG2D ligand, a chimeric polypeptide, or a transcriptional activated CAR trigger by the binding of the chimeric polypeptide to a target molecule (e.g., NKG2D-CAR), a modified TCR, an exogenous cytokine and/or a therapeutic monoclonal antibody of the present application, which may optionally be comprised in one or more vectors.

In the present application, the methods described in the present application can also be interpreted as therapeutic uses, that is, the methods described in the present application can be considered as therapeutic uses of the composition, nucleic acid molecules or engineered cells of the present application or pharmaceutical uses for preparing corresponding therapeutic uses. For example, the present application relates to the use of the above-mentioned engineered cells or nucleic acid molecules for regulating the activity of engineered cells or the use of the above-mentioned engineered cells for preparing medicaments for regulating the activity of engineered cells; the present application relates to the use of the above-mentioned engineered cells or nucleic acid molecules for activating engineered cells or the use of the above-mentioned engineered cells for preparing medicaments for activating engineered cells; the present application relates to the use of the above-mentioned engineered cells or nucleic acid molecules for inhibiting the activity of target cells in a subject or the use of the above-mentioned engineered cells or nucleic acid molecules for preparing medicaments for inhibiting the activity of target cells in a subject; the present application relates to the use of the above-mentioned engineered cells or nucleic acid molecules for improving or treating the health status of a subject in need thereof or the use of the above-mentioned engineered cells or nucleic acid molecules for preparing medicaments for improving or treating the health status of a subject in need thereof. All the above contents described in the present application can also be applied to therapeutic or pharmaceutical uses.

The present application provides methods for inducing and/or increasing an immune response in a subject in need of the composition of the present application. The composition of the present application can be used to treat and/or prevent tumors in a subject. The composition of the present application can be used to prolong the survival of a subject suffering from a tumor. The composition of the present application can also be used to treat and/or prevent pathogen infection or other infectious diseases in human subjects such as those with compromised immune function. Such methods comprise administering an effective amount of a composition of the present application to achieve the desired effect, whether alleviating an existing condition or preventing a recurrence. For therapeutic purposes, the amount administered will be that amount effective to produce the desired effect. One or more administrations may be used to provide an effective amount. An effective amount may be provided in large doses or by continuous infusion.

The present application provides a method for transducing a viral vector into a cell (e.g., an immune effector cell), involving activation and transduction of the cell to be transduced, the activation and transduction of the cell can be performed simultaneously, that is, an input composition comprising the cell to be transduced, a stimulator for the cell to be transduced, and a viral vector particle carrying the recombinant nucleic acid are co-incubated, or activation can be performed first and then transduction, such as incubating the input composition comprising the cell to be transduced and a stimulator for the cell to be transduced together for activation, and then adding the viral vector particles carrying the recombinant nucleic acid for incubation, and performing transduction activation and transduction of the recombinant nucleic acid. The total time is controlled to be completed within 72 hours, preferably, within 48 hours, or within 36 hours, or within 24 hours. For example, the methods provided involve incubating and/or contacting retroviral vector particles (e.g., lentiviral vectors) with a population of cells (e.g., immune cells, such as T cells), activating the T cells using an ex vivo cell activation reagent (e.g., an anti-CD3/anti-CD28 reagent) before and/or simultaneously with and/or after contacting or incubating the cells with the viral particles. Preferably, the cells are activated prior to viral transduction. For example, when the input composition comprising the cell to be transduced, the stimulator for the cell to be transduced, and the viral vector particle carrying the recombinant nucleic acid are incubated together, the incubation time is no more than 72 hours for harvesting to obtain the output composition, wherein the output composition comprises the cell transduced with the recombinant nucleic acid; preferably, the incubation time is 1 hour to 72 hours; more preferably, the incubation time is 2 hours to 48 hours; more preferably, the incubation time is 2 hours to 36 hours; more preferably, the incubation time is 12 hours to 36 hours; more preferably, the incubation time is 12 hours to 24 hours; more preferably, the incubation time is 15 hours to 24 hours. For example, after the output composition is purified by washing, and centrifugation, etc., pharmaceutical preparation can be prepared without further in vitro amplification and culture, that is, the medicament prepared using the output composition do not require in vitro amplification before being used in a subject (or a patient). For example, a method for transducing cells with a viral vector comprises the following steps: step (1), incubating an input composition containing the cell to be transduced and a stimulator for the cell to be transduced for no more than 72 hours, step (2), adding a viral vector particle containing the recombinant nucleic acid and incubating for no more than 24 hours, and step (3) harvesting to obtain an output composition, wherein the output composition contains cells transduced with the recombinant nucleic acid; preferably, the total incubation time of (1) and (2) does not exceed 72 hours; more preferably, the total incubation time of (1) and (2) does not exceed 60 hours, or does not exceed 48 hours, or does not exceed 32 hours, or does not exceed 28 hours; more preferably, the total incubation time of (1) and (2) does not exceed 24 hours. For example, the incubation time of step (1) is 2-72 hours; preferably, the incubation time of step (1) is 2-71 hours; more preferably, the incubation time of step (1) is 2-48 hours; more preferably, the incubation time of step (1) is 2-32 hours; more preferably, the incubation time of step (1) is 2-28 hours; more preferably, the incubation time of step (1) is 3-24 hours; more preferably, the incubation time of step (1) is 5-24 hours; more preferably, the incubation time of step (1) is 7-24 hours; more preferably, the incubation time of step (1) is 7-23 hours; more preferably, the incubation time of step (1) is 10-23 hours; more preferably, the incubation time of step (1) is 15-23 hours; more preferably, the incubation time of step (1) is 15-22 hours. For example, the incubation time of step (2) is 30 minutes to 24 hours, preferably, the incubation time of step (2) is 30 minutes to 21 hours; preferably, the incubation time of step (2) is 30 minutes to 17 hours; preferably, the incubation time of step (2) is 30 minutes to 12 hours; preferably, the incubation time of step (2) is 30 minutes to 10 hours; preferably, the incubation time of step (2) is 30 minutes to 8 hours; preferably, the incubation time of step (2) is 1 hour to 8 hours; preferably, the incubation time of step (2) is 1 hour to 4 hours; more preferably, the incubation time of step (2) is 1 hour to 3 hours. For example, the recombinant nucleic acid may encode a receptor that recognizes a tumor antigen or a pathogen antigen, such as a recombinant T cell receptor. The resulting cells transduced with the recombinant nucleic acid can be used for adoptive immunotherapy. For example, the provided methods can be used to prepare immune cells, such as T cells, for adoptive therapy, wherein the total activation and transduction time is controlled within 24 hours, or 36 hours, or 48 hours, or 72 hours. For example, the provided methods shorten the time to engineer and/or prepare cells for adoptive cell therapy. For example, the input composition comprises a primary cell population that has been obtained from a sample from a subject and/or enriched for a particular cell subset (e.g., T cells). For example, a cell population (e.g., an input composition) can be a cell population that has been previously cryopreserved. For example, incubating and/or contacting begins no more than or about no more than 1 hour, 3 hours, 6 hours, 12 hours, 18 hours, 24 hours, 48 hours, or 72 hours after obtaining the sample containing primary cells (e.g., an apheresis sample) from the subject. For example, the method produces an output composition, wherein at least 25%, at least 30%, at least 40%, at least 50% or at least 75% of the total cells (or a particular target cell type, such as T cells) in the output composition are transduced with the viral vector and/or express the recombinant gene product encoded thereby. For example, at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of the cells (e.g., T cells) in a cell population (e.g., an output composition) are transduced with retroviral vector particles according to the provided methods.

Activation or stimulation can be performed ex vivo or in vivo. For example, after incubation (e.g., transduction) of cells with viral particles, the cells can be infused into a patient for in vivo activation and expansion. For example, the cell activators to be transduced may be one, two, or a combination of more. For example, T cell activators can be CD3 binding molecules (such as CD3 antibodies), CD28 binding molecules (such as CD28 antibodies), recombinant IL-2, recombinant IL-15, recombinant IL-7, recombinant IL-21, or a mixture of at least two, such as CD3 antibodies and CD28 antibodies, or CD3 antibodies, CD28 antibodies or IL2. For example, the multiplicity of infection of the viral vector particles is not higher than 20; preferably, the multiplicity of infection is 0.5-20; more preferably, the multiplicity of infection is 1.5-20; more preferably, the multiplicity of infection is 3-20; more preferably, the multiplicity of infection is 3-12. For example, during or after the incubation, provided methods can further comprise culturing the input composition, output composition, and/or transduced cells ex vivo, such as under conditions that activate the cells to induce their proliferation and/or activation. Activation is carried out in the presence of one or more activators. For example, the activator can be a CD3 binding molecule, a CD28 binding molecule, or a cytokine (such as recombinant IL-2, recombinant IL-15, recombinant IL-7, or recombinant IL-21). For example, the binding molecule is an antibody or antigen binding fragment, such as an anti-CD3 antibody and/or an anti-CD28 antibody. For example, further culturing is performed under conditions that achieve expansion of the cells to produce a therapeutically effective amount of the cells for administration to a subject via adoptive cell therapy. For example, the provided methods avoid significantly altering the differentiation state of T cells ex vivo and/or minimize changes in the differentiation state of T cells during the process of introducing, transferring and/or transducing T cells with nucleic acids encoding recombinant receptors (e.g., CARs). For example, memory T cells are generated according to the provided methods, comprising stem cell memory T cells, central memory T cells, and effector memory T cells. For example, in the output composition of the cells of the present application, the proportion of cells transduced with recombinant nucleic acids (such as CAR T cells) is lower than that in the conventional process. In a specific embodiment, the number of cells does not exceed 1×10¹⁰, 1×10⁹, 1×10⁸, 1×10⁷, 1×10⁶, 1×10⁵ or 1×10⁴. For example, the content of cells with memory cell phenotype (such as memory T cells) in the output composition of the present application comprising cells transduced with recombinant nucleic acid is higher than that in conventional processes. For example, the content is at least 1.5 times, 2 times, 3 times, 4 times or 5 times higher. For example, memory T cells are cells having a T central cell memory (TCM) phenotype, such as CD45RO+CCR7+CD62L+T cells and/or CD45RO+CCR7+CD27+CD28+CD62L+T cells. For example, one, multiple or all steps in the preparation of cells for clinical use (e.g., adoptive cell therapy) of the present application are performed under sterile conditions. For example, one or more of the processes in which cells are enriched, activated, transduced, or washed is performed in a closed system. For example, cells can be treated ex vivo for a shorter period of time to further reduce the time. For example, cells transduced with recombinant nucleic acids (e.g., CAR T cells) produced by the provided methods exhibit longer persistence and/or reduced cell consumption when administered to a subject. For example, cells transduced with recombinant nucleic acids (such as CAR T cells) produced by the provided methods exhibit improved efficacy when administered to a subject. For example, the provided methods reduce the variability of cells during the preparation of cell therapy products. For example, eliminating the time required for ex vivo activation and transduction of cells improves the process of preparing cells transduced with recombinant nucleic acid for adoptive immunotherapy.

Provided herein is a method of incubating or contacting an input composition (comprising cells to be transduced) with retroviral vector particles (e.g., lentiviral vector particles). For example, the input composition is a composition of primary cells obtained from a subject, wherein, in some cases, a subpopulation or subset of cells has been selected and/or enriched. For example, when the cells to be transduced are T cells, the input composition can be a T cell population, an enriched T cell population, or PBMCs.

In one embodiment, the method comprises processing steps performed in the following order, wherein: first, isolating (e.g., selecting or separating) primary cells from a biological sample; activating, expanding or proliferating the selected cells in vitro in the presence of an activation agent, and then transducing by incubating with viral vector particles, wherein the total activation and transduction time does not exceed or does not exceed about 24 or 36 or 48 hours, with the transduction time being at least about 1, 2, 3, 4, 5, 6, 7, 8, 9, 10, 11, 12, 13, 14, 15, 16, 17, 18, 19, 20, 21, 22, 23, or 24 hours.

The T cell activator is a solid support (e.g., beads, comprising magnetic beads and/or microbeads; polymer matrix, comprising polymer nanomatrix) coupled to anti-CD3 and/or anti-CD28 and/or anti-41-BB monoclonal antibodies. For example, a sample or composition of cells to be separated is incubated with small magnetizable or magnetically responsive materials such as magnetically responsive particles or microparticles, such as paramagnetic beads (e.g., such as Dynabeads or MACS beads). For example, the magnetic particles or beads comprise a magnetically responsive material bound to a specific binding member, such as an antibody or other binding partner. There are many well-known magnetically responsive materials used in magnetic separation methods. The incubation is typically performed under conditions whereby the antibody, or binding partner, or a molecule that specifically binds to such an antibody, or binding partner attached to magnetic particles or microbeads (such as a secondary antibody or other reagent) specifically binds to the cell surface molecule (if present on cells within the sample). In some aspects, the sample is placed in a magnetic field, and those cells that have magnetically responsive or magnetizable particles attached thereto will be attracted to the magnet and separated from unlabeled cells. For positive selection, cells attracted by the magnet are retained; for negative selection, cells not attracted (unlabeled cells) are retained. For example, the magnetically responsive particles or microbeads remain attached to the cells, which are subsequently incubated, cultured and/or engineered; in some aspects, the particles or microbeads remain attached to the cells for administration to a patient. For example, the magnetizable or magnetically responsive particles are removed from the cells. Methods for removing magnetizable particles or microbeads from cells are known and comprise, for example, the use of competing non-labeled antibodies and magnetizable particles or antibodies or microbeads conjugated to a cleavable linker. For example, the magnetizable particles are biodegradable.

Activation and/or expansion can be performed prior to or concurrently with cell transduction. For example, the methods provided herein do not comprise further culturing or incubation, for example, do not comprise an ex vivo expansion step, or comprise a significantly shorter ex vivo expansion step. For example, the entire process of engineering the cells (e.g., selection and/or enrichment, incubation combined with activation transduction, and/or further culturing or cultivating) is performed within the following time period after obtaining the sample from the subject: no more than 9 days, no more than 8 days, no more than 7 days, no more than 6 days, no more than 5 days, no more than 4 days, no more than 3 days, no more than 2 days, or no more than 1 day. It will be appreciated that the period of culturing or incubating may not comprise any period of time during which the cells are subjected to cryopreservation.

Engineered cells (e.g., an output composition or a formulated composition) can also be administered to a subject immediately or shortly after transduction without significant ex vivo expansion. For example, the engineered cells can be administered immediately after the transduction step. For example, engineered cells can be administered shortly after an activation transduction step, without significant ex vivo expansion or with significantly shorter ex vivo expansion than, for example, conventional methods (which may require significant in vitro activation, expansion and/or enrichment). For example, the engineered cells can be administered within three days, two days, or one day of transduction. For example, the engineered cells can be administered within 48 hours, 36 hours, 24 hours, 20 hours, 16 hours, 12 hours, 8 hours, 4 hours, 2 hours, 1 hour, or less of the activation transduction step. For example, the engineered cells are subjected to significantly shorter in vitro expansion times compared to conventional methods, e.g., 48 hours, 36 hours, 24 hours, 20 hours, 16 hours, 12 hours, 8 hours, 4 hours, 2 hours, 1 hour, or less. For example, expansion and/or activation of the cells can be performed in vivo following exposure to an antigen, e.g., expansion of the engineered cells can be performed in vivo in a subject following administration of the cells. For example, the extent, degree or magnitude of in vivo expansion can be expanded, enhanced or increased by a variety of methods that can modulate (e.g., increase) the expansion, proliferation, survival and/or efficacy of administered cells (e.g., cells expressing an exogenous gene).

The present application provides an antibody that specifically binds to MOG, which is a fully human antibody with low immunogenicity and few clinical side effects.

The term “MOG” is myelin oligodendrocyte glycoprotein. MOG is specifically expressed in oligodendrocytes of the mammalian central nervous system and is a component of the myelin sheath of the central nervous system. As used herein, “MOG” comprises any naturally occurring MOG from any vertebrate source, comprising mammals such as primates (e.g., humans and monkeys) and rodents (e.g., mice and rats). “MOG” refers to the MOG gene or any variant, derivative or isoform of the protein encoded by the gene. The human MOG polypeptide has an amino acid sequence or a fragment thereof having at least about 80%, at least about 85%, at least about 90%, at least about 95%, at least about 96%, at least about 97%, at least about 98%, at least about 99% or 100% homology or identity with the amino acid sequence encoded by the transcript expressed by the gene of NCBI GenBank Gene ID: 4340, and/or may optionally comprise at most one, at most two, or at most three conservative amino acid substitutions. The mouse MOG polypeptide has an amino acid sequence or a fragment thereof is at least about 80%, at least about 85%, at least about 90%, at least about 95%, at least about 96%, at least about 97%, at least about 98%, at least about 99% or 100% homology or identity with the amino acid sequence encoded by the transcript expressed by the gene of NCBI GenBank Gene ID: 17441, and/or may optionally comprise at most one, at most two, or at most three conservative amino acid substitutions. The term comprises “full-length,” unprocessed MOG as well as any form of MOG that results from processing in the cell. The term also comprises naturally occurring variants of MOG, such as splice variants or allelic variants. The anti-MOG antibodies described herein can specifically bind to human MOG. Anti-MOG antibodies can also specifically bind to human MOG and mouse MOG. Exemplarily, the full-length amino acid sequence of human MOG is represented by SEQ ID NO: 41.

Antibodies of the present application can be isolated by screening combinatorial libraries for antibodies possessing the desired activity or activities. For example, a variety of methods are known in the art for generating phage display libraries and screening such libraries for antibodies with desired binding properties.

In certain phage display methods, the VH and VL gene repertoires are cloned separately by polymerase chain reaction (PCR) and randomly recombined in a phage library, which can then be screened for phage binding to the antigen. Phage typically display antibody fragments as single-chain Fv (scFv) fragments or Fab fragments. Libraries from immunized sources provide high affinity antibodies to the immunogen without the need to construct hybridomas. Alternatively, the naive repertoire (e.g., from humans) can be cloned to provide a single source of antibodies to a variety of non-self antigens as well as self antigens without the need for any immunization. Finally, naive libraries can also be prepared synthetically by cloning unrearranged V-gene segments from stem cells and using PCR primers containing random sequences to encode the hypervariable CDR3 domains and achieve rearrangement in vitro.

Herein, antibodies or antibody fragments screened from a fully human antibody library are considered to be fully human antibodies or fully human antibody fragments.

The parent antibody may refer to the original antibody screened from an antibody library or hybridoma, may refer to the antibody used for antibody modification, or may refer to the antibody that needs to be modified.

The term “affinity-matured” antibody refers to an antibody with one or more alterations in one or more hypervariable domains (HVRs) compared to a parent antibody, which alterations improve the affinity of the antibody for antigen.

The term “variant” refers to a polypeptide having substantially the same amino acid sequence as the sequence of an antibody provided herein, or having one or more activities encoded by substantially the same nucleotide sequence. The variant has the same or similar activity as the antibody provided in the examples of the present application.

The term “variant antibody” or “antibody variant” encompasses antibodies that differ from a parent antibody by at least one amino acid modification. The amino acid sequence of variant antibody herein preferably has at least about 80%, 81%, 82%, 83%, 84%, 85%, 86%, 87%, 88%, 89%, 90%, 91%, 92%, 93%, 94%, 95%, 96%, 97%, 98%, 99% identity with the amino acid sequence of the parent antibody. An antibody variant may refer to the antibody itself, or may refer to a composition comprising the antibody variant. The amino acid sequence of the antibody variant can be prepared by introducing appropriate modifications into the nucleotide sequence encoding the antibody or by peptide synthesis.

The terms “anti-MOG antibody”, “antibody that binds to MOG”, “MOG antibody”, “antibody that recognizes MOG” refer to an antibody that can bind to MOG with sufficient affinity, which can be used to prepare diagnostic agents and/or therapeutic agents targeting MOG. An anti-MOG antibody may bind to an unrelated, non-MOG protein to an extent that is less than about 10% of the binding of the antibody to MOG, as determined by enzyme-linked immunosorbent assay (ELISA). Anti-MOG antibodies bind to an epitope of MOG that may be conserved between MOG derived from different species.

Antigen binding proteins having Fab (antigen binding fragment) based antigen binding domains are described herein, comprising antibody Fabs. The human MOG extracellular domain was used and Fab was selected from a fully human naive Fab phage library. These molecules display exquisite specificity. For example, this antibody only recognizes 293T cells overexpressing MOG but not wild-type 293T cells. Unless otherwise specified in the present application, MOG refers to human or mouse MOG.

The present application comprises antibodies having Fab or scFv sequences fused with one or more heavy chain constant domains to form antibodies having human immunoglobulin Fc domains to produce bivalent proteins, thereby increasing the overall affinity and stability of the antibodies. In addition, the Fc portion allows the direct conjugation of other molecules (including but not limited to fluorescent dyes, cytotoxins, radioisotopes, etc.) to the antibodies for use, for example, in antigen quantification studies, to immobilize antibodies for affinity measurements, for targeted delivery of therapeutics, testing of Fc-mediated cytotoxicity using immune effector cells, and many other applications.

The results presented herein highlight the specificity, sensitivity and utility of the antibodies of the present application in targeting MOG.

The antibodies or antibody fragments of the present application are based on the use of phage display to identify and select antigen-binding fragments (Fab), the amino acid sequence of which confers specificity to MOG on the antibody or antibody fragment and forms the basis of the antigen-binding protein. Therefore, the Fab can be used to design a series of different “antibodies or antibody fragments”, comprising, for example, full-length antibodies, fragments thereof such as F(ab′)2, fusion proteins, IgG4, multivalent antibodies (multifunctional antibodies), i.e., antibodies with more than one specificity for the same antigen or different antigens, for example, bispecific T cell-binding antibodies (BiTEs), triabodies, etc.

The present application also provides full-length antibodies, whose heavy and light chains can be full-length (e.g., the antibody can comprise at least one, preferably two complete heavy chains, and at least one, preferably two complete light chains) or can comprise an antigen binding portion (Fab, F(ab′)2, Fv or scFv). The antibody heavy chain constant domain can be selected from, for example, IgG1, IgG2, IgG3, IgG4, IgM, IgAQ1, IgA2, IgD or IgE. The choice of antibody type will depend on the immune effector functions that the antibody is designed to elicit. When constructing recombinant immunoglobulins, appropriate amino acid sequences for the constant domains of various immunoglobulin isotypes and methods for producing a wide variety of antibodies are known to those of skill in the art.

The present application provides an antibody that recognizes MOG, the antibody comprises a light chain variable domain, the light chain variable domain comprises LCDR1 represented by SEQ ID NO: 84, and/or LCDR2 represented by SEQ ID NO: 85, and/or LCDR3 represented by any one of SEQ ID NOs: 86, 87, 88, 89, 90, 91, 92, 93, 94, 95, 96, 97 or 98; and/or, the antibody comprises a heavy chain variable domain, the heavy chain variable domain comprises HCDR1 represented by SEQ ID NO: 99 or 100, and/or HCDR2 represented by SEQ ID NO: 101 or 102, and/or HCDR3 represented by any one of SEQ ID NOs: 103, 104, 105, 106, 107, 108, 109, 110, 111, 112, 113, 114 or 115. More preferably, the antibody that recognizes MOG comprises LCDR1, LCDR2, LCDR3, HCDR1, HCDR2, and HCDR3 respectively represented by SEQ ID NO: 84, 85, 86, 99, 101, and 103; or respectively represented by SEQ ID NO: 84, 85, 87, 99, 101, and 104; or respectively represented by SEQ ID NO: 84, 85, 88, 99, 101, and 105; or respectively represented by SEQ ID NO: 84, 85, 89, 99, 101, and 106; or respectively represented by SEQ ID NO: 84, 85, 90, 99, 101, and 107; or represented by SEQ ID NO: 84, 85, 91, 100, 102, and 108; or respectively represented by SEQ ID NO: 84, 85, 92, 100, 102, and 109; or respectively represented by SEQ ID NO: NO: 84, 85, 93, 100, 102, and 110; or respectively represented by SEQ ID NO:84, 85, 94, 100, 102, and 111; or respectively represented by SEQ ID NO: 84, 85, 95, 100, 102, and 112; or respectively represented by SEQ ID NO:84, 85, 96, 100, 102, and 113; or respectively represented by SEQ ID NO:84, 85, 97, 100, 102, and 114; or respectively represented by SEQ ID NO:84, 85, 98, 100, 102, and 115.

The present application provides an antibody that recognizes MOG, comprising a light chain variable domain that may comprise an amino acid sequence represented by SEQ ID NO: 116, 117, 118, 119, 120, 121, 122, 123, 124, 125, 126, 127 or 128, or a nucleic acid sequence encoding the amino acid sequence, or an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 100% identity with the above amino acid sequence, or a variant of the above amino acid sequence; and/or comprising a heavy chain variable domain comprising an amino acid sequence represented by NO: 129, 130, 131, 132, 133, 134, 135, 136, 137, 138, 139, 140 or 141, or a nucleic acid sequence encoding the amino acid sequence, or an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 100% identity with the above amino acid sequence, or a variant of the above amino acid sequence.

Given that each of these heavy chain variable domain sequences and light chain variable domain sequences can bind to MOG, the heavy chain and light chain variable domain sequences can be “mixed and matched” to generate the anti-MOG binding molecules of the present application. The light chain variable domain and heavy chain variable domain of the antibody that recognizes MOG respectively represented by the amino acid sequences: SEQ ID NO: 116 and 129; SEQ ID NO: 117 and 130; SEQ ID NO: 118 and 131; SEQ ID NO: 119 and 132; SEQ ID NO: 120 and 133; SEQ ID NO: 121 and 134; SEQ ID NO: 122 and 135; SEQ ID NO: 123 and 136; SEQ ID NO: 124 and 137; SEQ ID NO: 125 and 138; SEQ ID NO: 126 and 139; SEQ ID NO: 127 and 140; or SEQ ID NO:128 and 41, or nucleic acid sequences encoding the amino acid sequences, or amino acid sequences each having at least 80%, 81%, 82%, 83%, 84%, 85%, 86%, 87%, 88%, 89%, 90%, 91%, 92%, 93%, 94%, 95%, 96%, 97%, 98%, 99%, or 100% identity with the above amino acid sequences, or a variant of the above amino acid sequence.

The present application provides antibodies that recognize MOG, comprising scFv, which may comprise the amino acid sequence represented by SEQ ID NO: 43, 68, 77, 81, 82 or 83, or a nucleic acid sequence encoding the amino acid sequence, or an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 100% identity with the above amino acid sequence, or a variant of the above amino acid sequence.

The present application provides an antibody that recognizes the same antigenic determinant as the aforementioned anti-MOG antibody.

The present application provides an antibody that specifically binds to MOG, which may be a whole antibody (or “full antibody”, “complete antibody”), scFv, a single domain antibody, a Fab fragment, a Fab′ fragment, a Fv fragment, a F(ab′)2 fragment, a Fd fragment, a dAb fragment, a multifunctional antibody, a scFv-Fc antibody or an IgG4 antibody. The antibody binds to cells expressing MOG.

### Antibody Assay

The anti-MOG antibodies provided herein can be identified, screened, or characterized for their physical/chemical properties and/or biological activities by a variety of assays known in the art. These comprise, for example, ELISA, Biacore, Western blotting, and flow cytometric analysis. Suitable assays are described in detail in the Examples.

The term “affinity” refers to the sum of the strength of non-covalent interactions between a single binding site of a molecule (e.g., an antibody) and its binding partner (e.g., an antigen). Unless otherwise indicated, “binding affinity” as used herein refers to intrinsic binding affinity, which reflects a 1:1 interaction between members of a binding pair (e.g., antibody and antigen). The affinity of a molecule X for its ligand Y can generally be represented by the dissociation constant (Kd). Affinity can be measured by routine methods known in the art, including determining the affinity of an antibody using Biacore as described herein. The “affinity” of an antibody for MOG herein is represented as the KD of the antibody. The KD of an antibody refers to the equilibrium dissociation constant of the antibody-antigen interaction. The larger the KD value of an antibody binding to its antigen, the weaker its binding affinity for that specific antigen.

The term “EC50”, i.e., half maximal effect concentration, refers to the concentration that causes 50% of the maximal effect.

### Immunoconjugates

The present application also provides an immunoconjugate, wherein the immunoconjugate comprises the antibody described herein, and a functional molecule linked thereto. The antibody and the functional molecule can form a conjugate by covalent bonding, coupling, attachment, cross-linking, etc.

The terms “linked”, “connected” or “fused” are used interchangeably herein. These terms refer to the connection of two or more chemical elements or components by any means including chemical conjugation or recombinant methods. “In-frame fusion” refers to the connection of two or more open reading frames (ORFs) to form a continuous longer ORF in a manner that maintains the correct reading frame of the original ORFs. Thus, the resulting recombinant fusion protein is a single protein comprising two or more fragments corresponding to the polypeptides encoded by the original ORFs (these fragments are not normally so linked in nature). Although the reading frame is thus continuous throughout the fused fragments, the fragments may be physically or spatially separated by, for example, in-frame linker sequences (e.g., “flexons”).

Another aspect of the present application provides a nucleic acid molecule encoding at least one antibody of the present application, a functional variant thereof or an immunoconjugate thereof. Once the relevant sequence is obtained, the recombinant method can be used to obtain the relevant sequence in large quantities. This is usually done by cloning it into a vector, then transferring it into cells, and then isolating the relevant sequence from the proliferated host cells by conventional methods.

The present application also relates to a vector comprising the above-mentioned appropriate DNA sequence and an appropriate promoter or control sequence. These vectors can be used to transduce appropriate host cells to enable them to express proteins. The host cell can be a prokaryotic cell, such as a bacterial cell; or a lower eukaryotic cell, such as a yeast cell; or a higher eukaryotic cell, such as a mammalian cell.

Chimeric polypeptides MOG-synE or MOG-synEdel3 are constructed by respectively fusing each of A1-scFv, A2-scFv, A3-scFv, A4-scFv, A5-scFv, A6-scFv, A7-scFv, A8-scFv, A9-scFv, A10-scFv, A11-scFv, A12-scFv, or A13-scFv sequentially with an EphrinB2 extracellular domain or EphrinB2-del3 extracellular domain, a Notch transmembrane domain, and GAL4-VP64.

The present application provides one or more vectors (e.g., expression vectors) comprising the above nucleic acid, and host cells comprising the above nucleic acid. The host cell comprises (e.g., is transduced with): (1) a vector comprising nucleic acids encoding an amino acid sequence comprising the VL of the antibody and an amino acid sequence comprising the VH of the antibody, or (2) a first vector comprising a nucleic acid encoding an amino acid sequence comprising the VL of the antibody and a second vector comprising a nucleic acid encoding an amino acid sequence comprising the VH of the antibody. The host cell can be eukaryotic, for example, a Chinese hamster ovary (CHO) cell or a lymphocyte (e.g., YO, NSO, or Sp20 cell).

The host cell expresses the MOG-targeted chimeric polypeptide described in the present application.

The host cell may comprise a T cell, a natural killer cell, a cytotoxic T lymphocyte, a natural killer T cell, a DNT cell, a regulatory T cell, a NK92 cell, and/or a stem cell-derived immune effector cell.

The present application provides a method for preparing an anti-MOG antibody, wherein the method comprises culturing a host cell comprising a nucleic acid encoding the antibody under conditions suitable for expressing the antibody as described above, and optionally recovering the antibody from the host cell (or host cell culture medium).

In order to express the protein, the nucleic acid encoding the antibody of the present application can be integrated into an expression vector. A variety of expression vectors are available for protein expression. Expression vectors may comprise self-replicating extrachromosomal vectors, or vectors that integrate into the host genome. Expression vectors useful in the present application include, but are not limited to, those that enable protein expression in mammalian cells, bacteria, insect cells, yeast, and in vitro systems. As is known in the art, a variety of expression vectors are available commercially or otherwise. Can be used in the present application to express antibodies.

The host cells provided herein can be administered in combination with an agent that enhances their function, preferably, in combination with a chemotherapeutic agent; and/or the host cells can be administered in combination with an agent that mitigates one or more side effects associated therewith; and/or the host cells can be administered in combination with a host cell expressing a chimeric antigen receptor targeting other than MOG.

It is appreciated that certain features of the application, which are, for clarity, described in the context of separate embodiments, may also be provided in combination in a single embodiment. Conversely, various features of the application which are, for brevity, described in the context of a single embodiment, may also be provided separately or in any suitable subcombination. All combinations of embodiments of the present application are expressly included in the present application and disclosed herein, just as if each and every combination was individually and expressly disclosed. Furthermore, all subcombinations of the various embodiments and elements thereof are also expressly included in the present application and disclosed herein, just as if each and every such subcombination were individually and expressly disclosed herein.

The biological materials used in the examples of this application are shown in Table 2.

TABLE 2

| Name | Source |
|---|---|
| T cells | Isolated from primary PBMCs of healthy donors |
| NK cells | Isolated from primary PBMCs of healthy donors |
| Monocytes | Isolated from primary PBMCs of healthy donors |
| Jurkat cells | Cell Bank of Chinese Academy of Sciences (CAS) |
| SK-Hep1 | ATCC, HTB-52 |
| HuH7 | Cell Bank of CAS, TCHu182 |
| PLC/PRF/5 | ATCC, CRL-8024 |
| K562 | Cell Bank of CAS, TCHu191 |
| 293T | ATCC, CRL-11268 |
| U251 | Cell Bank of CAS, TCHu58 |
| U87 | Cell Bank of CAS, TCHu138 |
| THP-1 | Cell Bank of CAS, SCSP-567 |
| KG-1 | ATCC, CCL246 |
| Molm-13 | Nanjing Cobioer Biosciences Co., Ltd., CBP60678 |
| HL60 | Cell Bank of CAS, TCHu23 |
| MV-4-11 | Cell Bank of CAS, SCSP-5031 |
| NPG mice | Beijing Vitalstar Biotechnology Co., Ltd., genotype: |
| | NOD.Cg-Prkdc^{scid} Il2rg^{tm1Vst}/Vst |
| NOG mice | Beijing Vital River Laboratory Animal Technology Co., |
| | Ltd., genotype: NOD.Cg-Prkdc^{scid}IL2rg^{tm1Sug}/JicCrl |

### Example 1. Construction of Chimeric Polypeptide synE and its Mutants, Truncations and Modified Bodies Thereof

This example constructs the composition of various exemplary chimeric polypeptides used in this application (see, Table 3).

TABLE 3

| | | Receptor Regulatory Domain | | |
|---|---|---|---|---|
| Vector/Chimeric | | | Transmembrane | Intracellular |
| Polypeptide Name | Binding Domain | Extracellular Domain | Region | Domain |
| GPC3-synE | antiGPC3-scFv | EphrinB2 | Notch1 | GAL4-VP64 |
| GPC3-synE-del1 | antiGPC3-scFv | EphrinB2-del1 | Notch1 | GAL4-VP64 |
| GPC3-synE-del2 | antiGPC3-scFv | EphrinB2-del2 | Notch1 | GAL4-VP64 |
| GPC3-synE-del3 | antiGPC3-scFv | EphrinB2-del3 | Notch1 | GAL4-VP64 |
| GPC3-synE-del23 | antiGPC3-scFv | EphrinB2-del23 | Notch1 | GAL4-VP64 |
| GPC3-synE-EGF₁ | AntiGPC3-scFv | EphrinB2-(EGF-like domain)₁ | Notch1 | GAL4-VP64 |
| GPC3-synE-EGF₂ | AntiGPC3-scFv | EphrinB2-(EGF-like domain)₂ | Notch1 | GAL4-VP64 |
| GPC3-synE-EGF₃ | AntiGPC3-scFv | EphrinB2-(EGF-like domain)₃ | Notch1 | GAL4-VP64 |
| GPC3-synE-EGF₄ | AntiGPC3-scFv | EphrinB2-(EGF-like domain)₄ | Notch1 | GAL4-VP64 |
| MOG-synE | AntiMOG-scFv | EphrinB2 | Notch1 | GAL4-VP64 |
| MOG-synE-del3 | AntiMOG-scFv | EphrinB2-del3 | Notch1 | GAL4-VP64 |
| CD123-synE | AntiCD123-scFv | EphrinB2 | Notch1 | GAL4-VP64 |
| CD123-synE-del3 | AntiCD123-scFv | EphrinB2-del3 | Notch1 | GAL4-VP64 |
| CLDN18.2-synE | AntiCLDN18.2-scFv | EphrinB2 | Notch1 | GAL4-VP64 |
| GPRC5D-synE | AntiGPRC5D-scFv | EphrinB2 | Notch1 | GAL4-VP64 |
| CLL1-synE | AntiCLL1-scFv | EphrinB2 | Notch1 | GAL4-VP64 |

Exemplarily, the fragment sequences in Table 3 above are as follows: antiGPC3-scFv (SEQ ID No: 69), AntiCD123-scFv (SEQ ID No: 71), AntiMOG-scFv (SEQ ID No: 68), anti CLDN18.2-scFv (SEQ ID No: 75), AntiGPRC5D-scFv (SEQ ID No: 76), anti CLL1-scFv (SEQ ID No: 79), EphrinB2 extracellular domain (SEQ ID No: 1, or 2), EphrinB2-del1 (SEQ ID No: 3), EphrinB2-del2 (SEQ ID No: 4), EphrinB2-del3 (SEQ ID No: 5), EphrinB2-del23 (SEQ ID No: 6), EphrinB2-(EGF-like domain) 1 (SEQ ID No: 7), EphrinB2-(EGF-like domain) 2 (SEQ ID No: 8), EphrinB2-(EGF-like domain) 3 (SEQ ID No: 9), EphrinB2-(EGF-like domain) 4 (SEQ ID No: 10), Notch1 (SEQ ID No:15), Notch1 (SEQ ID No:15) and GAL4-VP64 (SEQ ID No:28).

The chimeric polypeptide expression vector was constructed using the pRRLSIN vector (Addgene).

After the chimeric polypeptide expression vectors were introduced into Jurkat cells, biotin-labeled antigen polypeptide (CARsgen Diagnostics, HD0832) and PE-labeled streptavidin (eBioscience, 12-4317-87) were used. The flow cytometry results showed that the chimeric polypeptides were stably expressed on the cell membrane.

BFP (genebank ID: QJR97815.1) was inserted downstream of the UAS-CMV promoter (SEQ ID NO: 30) regulated by Gal4-VP64 in Table 3 as a reporter gene for the activation of the chimeric polypeptide. Green fluorescent protein (GFP) regulated by the pGK promoter (Addgene #79120, 7721-8220 bp) was inserted downstream of BFP as a transduction positive marker. The GFP protein sequence is shown in genebank ID: UDY80669.1. UAS-BFP-PGK-GFP was connected according to the nucleic acid sequence and integrated into the pRRLSIN expression vector, and lentivirus was prepared according to conventional molecular biological techniques and transduced into Jurkat cells to construct Jurkat responding cells.

### Example 2. Regulation of Gene Expression by synE and Truncations Thereof

SK-Hep1-GPC3 cells overexpressing human GPC3 were constructed using conventional molecular biological techniques.

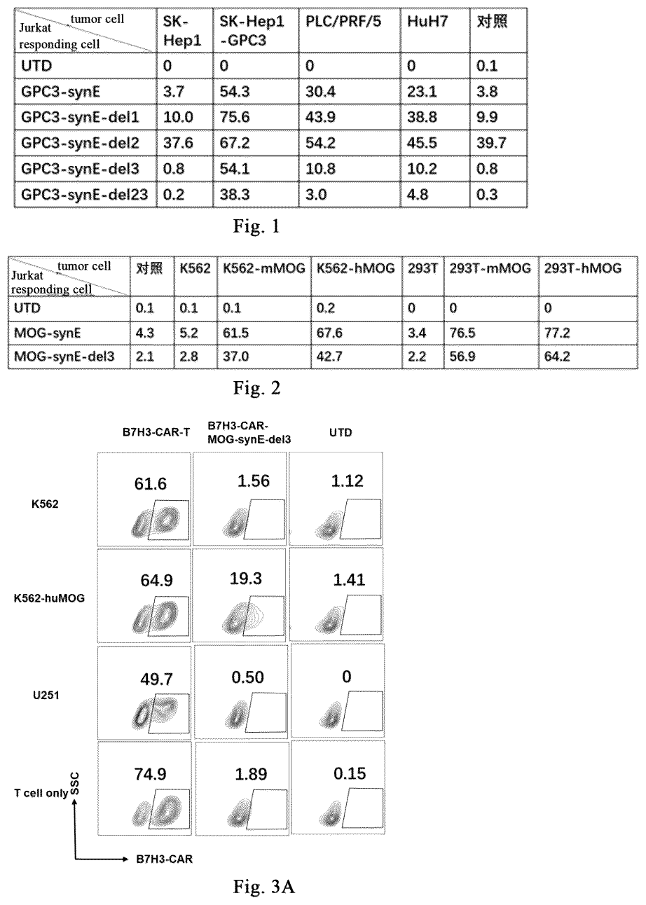

The Jurkat responding cells respectively containing GPC3-synE, GPC3-synE-del1, GPC3-synE-del2, GPC3-synE-del3, and GPC3-synE-del23 constructed in Example 1 were respectively mixed with 2×10⁴ liver cancer cells (SK-Hep1, SK-Hep1-GPC3, HuH7, or PLC/PRF/5) at a ratio of 1:1 and incubated at 37° C. for 24 h, and the BFP expression level was detected by flow cytometry. FIG. 1 shows that after co-incubation with GPC3-positive target cells (SK-Hep1-GPC3, HuH7, PLC/PRF/5), the chimeric polypeptides of GPC3-synE or truncations thereof all regulated gene expression; while in the absence of target cells (control), or after co-incubation with GPC3-negative target cells, synE and truncations thereof did not significantly induce BFP expression compared to the background level. The leakage of induced expression of synE-del3 or synE-del23 was significantly reduced.

### Example 3: MOG-synE Regulates Gene Expression

K562-mMOG, K562-huMOG, 293T-mMOG, or 293T-huMOG cells overexpressing human or mouse MOG were constructed using conventional molecular biological technique.

Jurkat responding cells containing chimeric polypeptides targeting MOG, MOG-synE and MOG-synE-del3, were respectively mixed with 2×10⁴ target cells at a ratio of 1:1 and incubated at 37° C. for 24 h. The expression level of BFP was detected by flow cytometry. FIG. 2 shows that after co-incubation with MOG-positive target cells (K562-mMOG, K562-huMOG, 293T-mMOG, or 293T-huMOG), MOG-synE or truncations thereof all regulated gene expression.

### Example 4. Regulatory B7H3-CAR-T Cells

B7H3-CAR (SEQ ID NO: 50) was inserted downstream of the UAS-CMV promoter (SEQ ID NO: 30), and then integrated into the chimeric polypeptide MOG-synE (SEQ ID NO: 46) expression vector (pRRLSIN) or the MOG-synE-del3 (SEQ ID NO: 47) expression vector (pRRLSIN) to construct B7H3-CAR-MOG-synE (SEQ ID NO: 58) vector or B7H3-CAR-MOG-synE-del3 (SEQ ID NO: 59) vector. The corresponding lentivirus of the above expression vector is prepared using conventional molecular biological techniques.

The conventional method for preparing CAR-T in the art was adopted: donor PBMC cells were collected, activated by magnetic beads (Life Technologies, 40203D) with anti-CD3 and CD28 antibodies, and then cultured to obtain T cells; after infecting T cells with a lentivirus comprising the above-mentioned vector, T cells expression of B7H3-CAR regulated by the chimeric polypeptide, i.e., regulatory B7H3-CAR-T cells, were prepared.

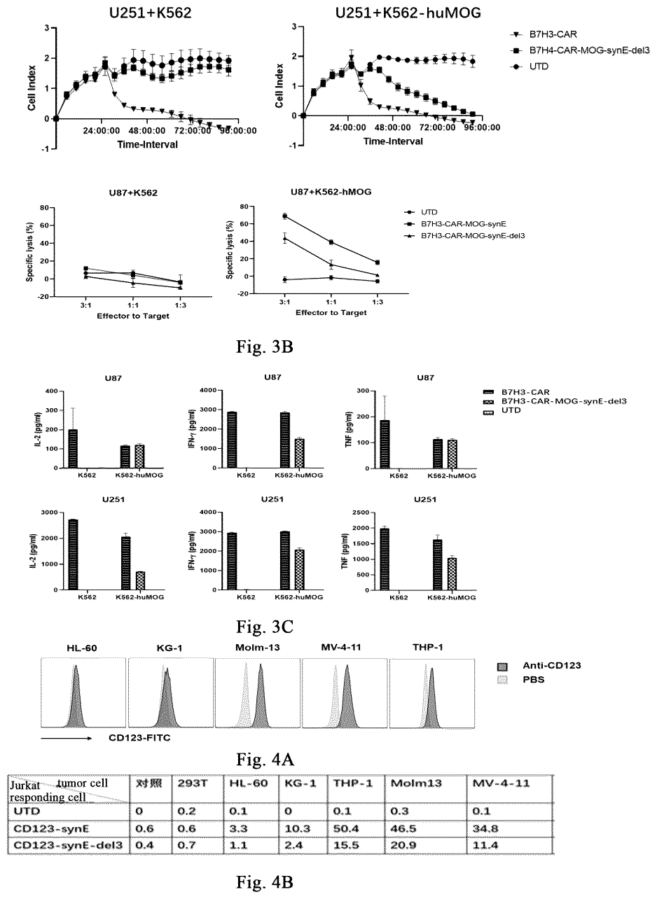

2×10⁴ of regulatory B7H3-CAR-T cells were mixed with target cells in a 1:1 ratio and incubated at 37° C. for 24 h. B7H3-CAR was labeled with antigen huB7-H3 (self-made antigen using conventional molecular biology techniques) and detected by flow cytometry. As shown in FIG. 3A, after the regulatory B7H3-CAR-T cells were co-incubated with K562-huMOG, the expression of B7H3-CAR was induced; after the regulatory B7H3-CAR-T cells were co-incubated with K562 or U251, B7H3-CAR was not expressed.

### Example 5. In Vitro Killing of Regulatory B7H3-CAR-T Cells

10,000 U251 cells were added to a 96-well E-plate (Agilent, 300600900). After 24 hours of adherence culture, 2,000 leukemia cells (K562, or K562-huMOG) and 10,000 regulatory B7H3-CAR-T cells were added, mixed, and incubated at 37° C. for 72 hours. During the incubation process, the cell growth index was recorded in real time (Agilent, xCELLigence RTCA MP) to detect the killing effect.

10,000 U87 cells: leukemia cells (K562, or K562-huMOG): regulatory B7H3-CAR-T cells were mixed in a ratio of 3:3:1 or 1:1:1 or 1:1:3 and incubated at 37° C., wherein U87 cells were target cells and T cells were effector cells, i.e., the effector-target ratio was 3:1, 1:1, and 1:3, respectively. After incubation at 37° C. for 24 hours, the killing effect was detected by the LDH method (Roche, 11644793001).

10,000 U87 or U251 cells: CAR-T cells: leukemia cells (K562, or K562-huMOG) were mixed at a ratio of 1:1:0.2, incubated at 37° C. for 24 h, and the supernatant was collected. Cytometric bead array (BD, 558264) was used to detect the levels of cytokines.

FIGS. 3B and 3C show that the regulatory B7H3-CAR-T cells can effectively kill B7H3-positive glioma cells U87 and U251 in the presence of MOG; however, in the absence of MOG, the killing effect on B7H3-positive glioma cells is weakened or there is no killing; the regulatory B7H3-CAR-T cells release the cytokine IL2 only in the presence of MOG.

### Example 6. CD123-synE Regulates Gene Expression

Jurkat responding cells comprising the chimeric polypeptide CD123-synE (SEQ ID NO: 48) or CD123-synE-del3 (SEQ ID NO: 49) targeting CD123 were respectively mixed with 2×10⁴ AML cells at a ratio of 1:1, incubated at 37° C. for 24 h, and the BFP expression level was detected by flow cytometry. FIGS. 4A and 4B show that after co-incubation with CD123-positive target cells (Molm13, MV-4-11, or THP-1), the chimeric polypeptides of CD123-synE or truncations thereof all regulated gene expression.

### Example 7. In Vitro Killing of Regulatory NKG2D-CAR-T Cells

ZBB-NKG2D-CAR (SEQ ID NO: 51), ZNKG2D-CAR-DAP10 (SEQ ID NO: 53), ZNKG2D-CAR (SEQ ID NO: 52), or NKG2D-28Z-CAR (SEQ ID NO: 54) were respectively inserted downstream of the UAS-CMV promoter (SEQ ID NO: 30), and then integrated into the chimeric polypeptide CD123-synE (SEQ ID NO: 48) expression vector or the CD123-synE-del3 (SEQ ID NO: 49) expression vector, and T cells were transduced to prepare NKG2D-CAR-T cells regulated by the chimeric polypeptide targeting CD123. Constitutive NKG2D-CAR-T cells were constructed using expression vectors (pRRLSIN) containing ZBB-NKG2D-CAR (SEQ ID NO: 51), ZNKG2D-CAR-DAP10 (SEQ ID NO: 53), ZNKG2D-CAR (SEQ ID NO: 52), or NKG2D-28Z-CAR (SEQ ID NO: 54), respectively.

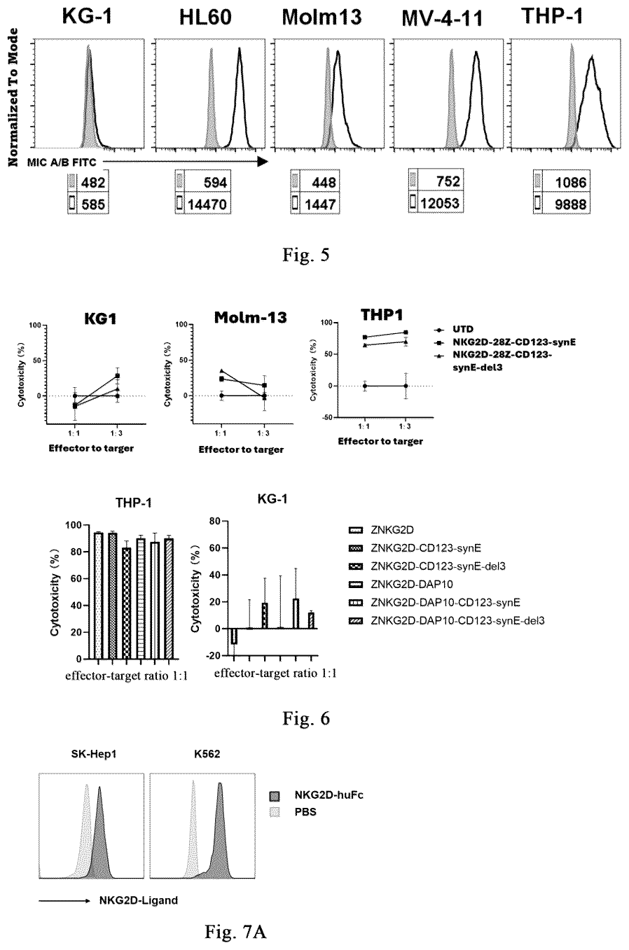

The CAR-T cells constructed above were mixed with 2×10⁴ AML cells at different effector-target ratios and co-cultured at 37° C. for 18 hours. The number of surviving target cells was detected by flow cytometry (7-AAD negative (BD, 559925), CD3 negative (eBioscience, 12-0038-42)). FIG. 6 shows that NKG2D-28Z-CAR-T cells, ZNKG2D-CAR or ZNKG2D-CAR-DAP10-T cells regulated by the chimeric polypeptide significantly killed THP1 cells expressing NKG2D ligands, but had weak killing effects on KG-1 and Molm 13 cells (FIG. 5) with low expression of NKG2D ligands.

This indicates that the killing ability of NKG2D-CAR-T cells regulated by CD123-synE or CD123-synE-del3 is NKG2D ligand-dependent, while high expression of CD123 alone (such as Molm13) will not cause NKG2D ligand-independent killing.

### Example 8. Specificity of Killing by Regulatory NKG2D-CAR-T Cells In Vitro

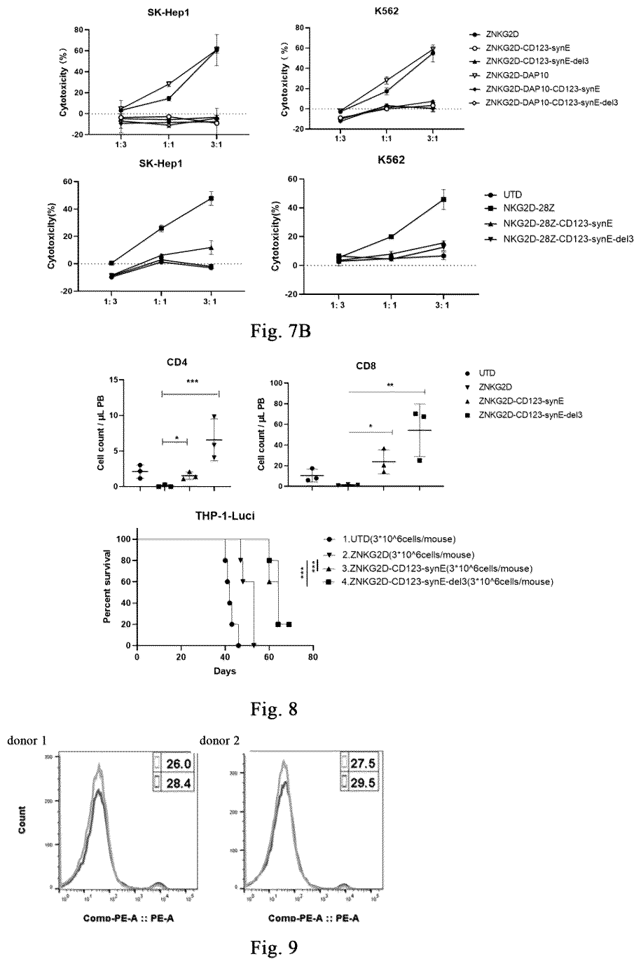

The regulatory NKG2D-CAR-T cells constructed in Example 7 were mixed with 1×10⁴ SK-Hep1 or K562 cells expressing NKG2D ligands but not expressing CD123 at different effector-target ratios, co-cultured at 37° C. for 18 hours, and supernatant LDH (Roche, 11644793001) was detected. FIGS. 7A and 7B show that constitutive NKG2D-CAR-T cells can effectively kill target cells, while regulatory NKG2D-CAR-T cells have almost no killing effect. This result indicates that CD123-synE or CD123-synE-del3 further enhances the specificity of NKG2D-CAR-T cells targeting AML cells.

### Example 9. In Vivo Survival and Therapeutic Effect of NKG2D-CAR-T Cells Expressing CD123-synE Chimeric Polypeptide

THP-1 cellsexpressing exogenous luciferase (THP1-Luci) were constructed using conventional molecular biological techniques.

Immunodeficient (NPG) mice were injected with 1×10⁷ THP-1-Luci cells per mouse through the tail vein. After tumor formation was confirmed by fluorescence imaging on the 15th day, they were divided into groups as shown in the figure, with 5 mice in each group. The corresponding number of NKG2D-CAR-T cells or UTD cells were intravenously infused, and fluorescence imaging of the tumor was performed weekly (IVIS LUMINA III SYSTEM). On the 9th day after the injection of CAR-T cells, peripheral blood of mice was collected and the number of surviving CD4+ and CD8+ T cells therein was detected quantitatively by flow cytometry (CD3 antibody (Thermo, 46-0036-42), CD4 antibody (BD, 562424), CD8 antibody (BD, 555369)). FIG. 8 shows that placing ZNKG2D-CAR under the regulation of CD123-synE or CD123-synE-del3 can improve the in vivo survival ability and anti-tumor effect of CAR-T cells.

### Example 10. Universal CAR (UCAR) T Cell Construction

The sgRNA sequences respectively targeting TRAC, B2M, or FAS (SEQ ID NOs: 142, 143, and 144) were synthesized in vitro (CARsgen Diagnostics), and the endogenous TCR/B2M or TCR/B2M/FAS of the regulatory NKG2D-CAR-T cells constructed in Example 7 were knocked out by CRPSP/Cas9 technology (Cas9 protein, Kactus Biosystems (Shanghai) Co., Ltd., CAS-EE109), and NKG2D-UCAR-T-DKO or NKG2D-UCAR-T-TKO were respectively obtained by magnetic bead sorting (Miltenyi Biotec, 130-048-801). Untransduced U-UTD cells with TCR/B2M knockout were used as controls. Constitutive NKG2D-UCAR-T cells were constructed in the same way.

### Example 11 the Expression of NKG2D-Ligand (NKG2DL) in Resting NK Cells from Different Donors was Detected by Flow Cytometry

PBMCs of the peripheral blood from different donors were collected, and NK cells were screened using CD56 magnetic beads (Miltenyi Biotec, 130-050-401) according to the manufacturer's instructions. 5×10⁵ NK cells were taken, NKG2D-Fc protein (final concentration: 5 μg/mL) (R&D, 1299-NK) was added, and PBS was used as a negative control, and incubated at room temperature for 15 minutes. Anti-human IgG Fc-PE fluorescent antibody (1:200 dilution) (eBioscience, 12-4998-82) was added to label the cells bound to NKG2D-Fc protein, and the expression of NKG2DL was detected by flow cytometry. FIG. 9 shows that NKG2DL is hardly expressed in resting NK cells from different donors.

### Example 12 the Expression of NKG2DLin NK Cells after Co-Culture with Tumor Cells, or UCAR-T Cells for 24 Hours was Detected by Flow Cytometry

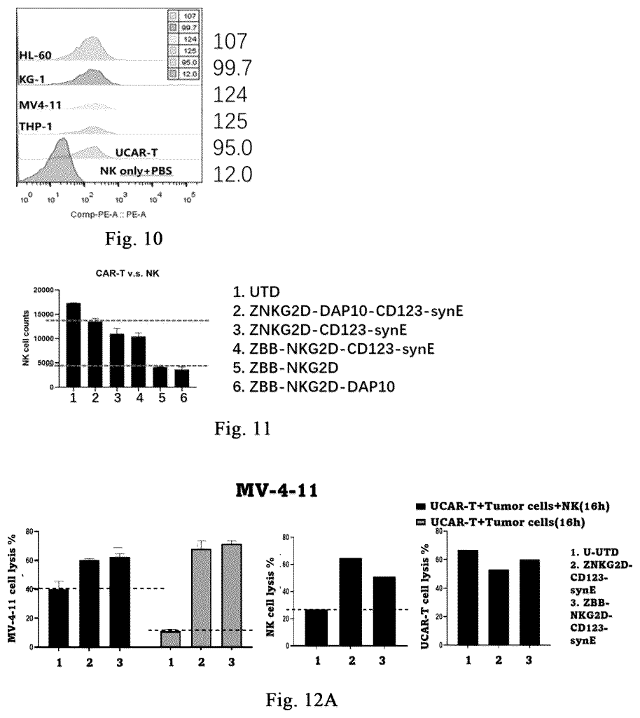

The human primary NK cells (3×10⁴) collected in Example 11 were inoculated with tumor cells (HL60, KG-1, MV4-11, and THP-1) at a ratio of 1:1, or 3×10⁴ human primary NK cells were inoculated with NKG2D-UCAR-T cells at a ratio of 1:1. After incubation at 37° C. for 24 hours, NKG2D-Fc protein (final concentration: 5 μg/mL) (R&D, 1299-NK) was added, and PBS was used as a negative control. The cells were incubated at room temperature for 15 minutes. Anti-human IgG Fc-PE fluorescent antibody (1:200 dilution) (eBioscience, 12-4998-82) was added to label the cells bound to NKG2D-Fc protein, and the expression of NKG2DL was detected by flow cytometry. FIG. 10 shows that the expression of NKG2DL on NK cells was up-regulated after NK cells were co-cultured with different tumor cells, or after NK cells were co-cultured with NKG2D-UCAR-T cells.

### Example 13. The Killing Effect of Regulatory or Constitutive NKG2D-UCAR-T (TRAC/B2M KO) Cells on NK Cells

2×10⁴ human primary NK cells were taken and inoculated with UTD or NKG2D-UCART cells (regulatory or constitutive) at an effector-target ratio of 1:1. After incubation at 37° C. for 16 hours, CD3-PE antibody (eBioscience, 12-0038-42) was used to label CAR-T cells, and CD56-APC (eBioscience, 17-0567-42) was used to label NK cells. 7-AAD fluorescent dye (BD, 559925) was used to distinguish dead cells from live cells, and the number of NK cells was detected by flow cytometry. FIG. 11 shows that compared with the UTD group, both regulatory and constitutive NKG2D-UCAR-T cells are able to kill NK cells.

### Example 14. Regulatory NKG2D-UCAR-T (TRAC/B2M KO) Cells Kill NK Cells

2×10⁴ human primary NK cells, tumor cells (MV-4-11 or MOLM-13 expressing both CD123 and NKG2DL) and UCAR-T cells, were mixed at a ratio of 1:1:1, and incubated at 37° C. for 16 h, 36 h or 72h. Then, HLA-ABC-PE (eBioscience, 12-9983-42) was used to label UCAR-T cells, and CD56-APC (eBioscience, 17-0567-42) was used to label NK cells; 7-AAD fluorescent dye (BD, 559925) was used to distinguish dead cells from live cells. Flow cytometry was used to detect the killing effect of UCAR-T cells on tumor cells, and the killing effect of UCAR-T cells on NK cells and NK cells on UCAR-T cells were also detected.

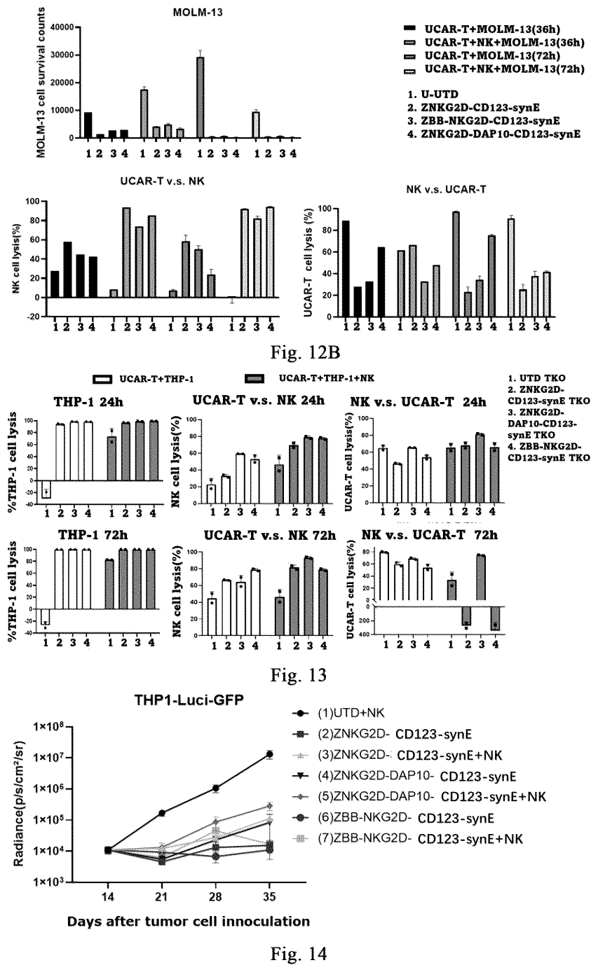

As shown in FIGS. 12A and 12B, in the absence of NK cells, the regulatory NKG2D-UCAR-T can significantly kill NKG2DL-positive tumor cells. When NK cells are present, regulatory NKG2D-UCAR-T cells can also significantly kill tumor cells, and the killing effect becomes stronger as the co-incubation time prolongs. After co-incubation with tumor cells, the regulatory NKG2D-UCAR-T cells have a strong killing effect on NK cells and can partially resist the killing effect of NK cells.

### Example 15. Regulatory NKG2D-UCAR-T (TRAC/B2M/Fas KO) Cells Kill NK Cells

2×10⁴ human primary NK cells, tumor cells (THP-1) and regulatory NKG2D-UCAR-T cells were mixed at a ratio of 1:1:1, and incubated at 37° C. for 24 h or 72h. Then, HLA-ABC-PE (eBioscience, 12-9983-42) was used to label UCAR-T cells, and CD56-APC (eBioscience, 17-0567-42) was used to label NK cells; 7-AAD fluorescent dye (BD, 559925) was used to distinguish dead cells from live cells. Flow cytometry was used to detect the killing effect of UCAR-T cells on tumor cells, and the killing effect of UCAR-T cells on NK cells and NK cells on UCAR-T cells were also detected. As shown in FIG. 13, in the absence of NK cells, regulatory NKG2D-UCAR-T cells can significantly kill NKG2DL-positive tumor cells. When NK cells are present, regulatory NKG2D-UCAR-T cells significantly kill tumor cells. After co-incubation with tumor cells, the regulatory NKG2D-UCAR-T cells have a strong killing effect on NK cells, and the longer the co-incubation time, the stronger the killing effect; and can partially resist the killing effect of NK cells.

### Example 16. In Vivo Antitumor Efficacy of Regulatory NKG2D-UCAR-T (TRAC/B2M/Fas KO) Cells

THP1-luci-GFP cells (8×10⁶/mouse) were inoculated into NOG mice in situ and divided into 7 groups, with 5 mice in each group. Fluorescence imaging (IVIS LUMINA III SYSTEM) was performed 14 days after inoculation. When the photon intensity reached an average of 1.1×10⁴p/s/cm²/sr, NK (2×10⁶/mouse) was injected intravenously for 3 consecutive days. After the first NK injection, the regulatory NKG2D-UCAR-T (5×10⁶/mouse) was injected. As shown in FIG. 14, 27 days after UCAR-T infusion, regardless of the presence or absence of NK cells, UCAR-T cells expressing ZNKG2D-CD123-synE, ZNKG2D-DAP10-CD123-synE, or ZBB-NKG2D-CD123-synE all can effectively inhibit the growth of leukemia xenografts, among which UCAR-T cells expressing ZBB-NKG2D-CD123-synE has the best efficacy.

### Example 17. Rapid Preparation of CAR-T Cells Targeting NKG2DL

After activation of T cells with magnetic beads (Miltenyi Biotec, 170-076-156) for 1, 2, 3, 4, 5, 6, 7, 8, 9, 10, 11, 12, 13, 14, 15, 16, 17, 18, 19, 20, 21, 22, 23 or 24 hours, they were transduced with a virus containing a regulatory or constitutive NKG2D-CAR (prepared by conventional molecular biology techniques). The transduction time was 1, 2, 3, 4, 5, 6, 7, 8, 9, 10, 11, 12, 13, 14, 15, 16, 17, 18, 19, 20, 21, 22, 23 or 24 hours. The transduced T cells were collected and cryopreserved, or the density of T cells were adjusted to about 1×10⁶ cells/ml and inoculated in a 24-well plate, and the culture was continued for more than 24 hours and cryopreserved.

### Example 18. Preparation of MOG Recombinant Protein

The fusion protein MOG_huFc formed by the extracellular segment of human MOG (positions 30-154 of the full length of human MOG represented by SEQ ID NO: 41) and the Fc segment of the human IgG1 heavy chain constant domain was synthesized in vitro. The MOG_huFc protein was obtained by transfecting 293F cells and expressing and purifying them using conventional protein purification technologies.

### Example 19. Screening and Identification of MOG Antibodies

### 1. Screening of MOG-Specific Antibodies Using a Fully Human Phage Display Library

The phage display library used in this application is a fully human natural scFv phage library constructed by our company, with a library capacity of 1E+11. Fab fragments highly specific for MOG were obtained using screening methods known to those skilled in the art.

Briefly, 10 μg/ml antigen MOG_huFc and human Fc fragment were coated on immunotubes respectively. In order to reduce the influence of the Fc fragment, the phage library was added to the immunotube coated with the human Fc fragment and bound for 1 hour. The supernatant was added to the immunotube coated with MOG_huFc and bound for 1.5 hours, and then the non-specific phages were washed away, and the bound phages were eluted and used to infect Escherichia coli TG1 in the logarithmic growth phase. The eluted phages were expanded and purified using PEG/NaCl precipitation for the next round of screening. Screening was performed for 3-4 cycles to enrich for Fab phage clones that specifically bind to MOG_huFc. Positive clones were identified by standard ELISA method against MOG_huFc. The specificity of the antibody was verified by ELISA using the human Fc fragment as an irrelevant antigen. A total of 376 clones were screened, of which 93 clones specifically bound to MOG_huFc but not to the human Fc segment. These 93 clones were retested by ELISA, and the results were highly consistent with the initial test results, of which 90 clones specifically bound to MOG_huFc but not to the human Fc segment. Among them, 47 clones with high signal values were selected for sequencing, and 13 clones were obtained, named A1, A2, A3, A4, A5, A6, A7, A8, A9, A10, A11, A12 and A13.

Among them, the LCDR1 and LCDR2 sequences of 13 clones were the same, represented by SEQ ID NO: 84 and 85, respectively; the HCDR1 and HCDR2 sequences of A1, A2, A3, A4, and A5 were the same, represented by SEQ ID NO: 99 and 101, respectively; the HCDR1 and HCDR2 sequences of A6, A7, A8, A9, A10, A11, A12, and A13 were the same, represented by SEQ ID NO: 100 and 102, respectively. The LCDR3 sequences of A1, A2, A3, A4, A5, A6, A7, A8, A9, A10, A11, A12, and A13 are represented by SEQ ID NOs: 86, 87, 88, 89, 90, 91, 92, 93, 94, 95, 96, 97, and 98, respectively; the HCDR3 sequences are represented by SEQ ID NOs: 103, 104, 105, 106, 107, 108, 109, 110, 111, 112, 113, 114, and 115, respectively; the VL sequences are represented by SEQ ID NOs: 116, 117, 118, 119, 120, 121, 122, 123, 124, 125, 126, 127, and 128, respectively; the VH sequences are represented by SEQ ID NOs: 129, 130, 131, 132, 133, 134, 135, 136, 137, 138, 139, 140, and 141, respectively. The scFv sequences of A2, A6, A8, A9, A10 and A13 are represented by SEQ ID NOs: 77, 81, 68, 82, 83 and 43, respectively.

### 2. The Specificity of Antibody Binding to Target Cells was Determined Using FACs

293T-hMOG cells and 293T-mMOG cells were counted and plated on U-bottom plates, with about 2×10⁵ cells in each well. They were incubated with primary antibodies (Fab forms A1, A2, A3, A4, A5, A6, A7, A8, A9, A10, All, A12 and A13: 5 μg/mL), respectively, and fluorescently labeled with secondary antibodies (Anti-Fc-FITC: 1:200, Jackson ImmunoResearch), and then the fluorescence intensity was detected using a flow cytometer. The experimental data were analyzed using FlowJo software to calculate the mean fluorescence intensity (MFI). The blank group NA was not added any antibody.

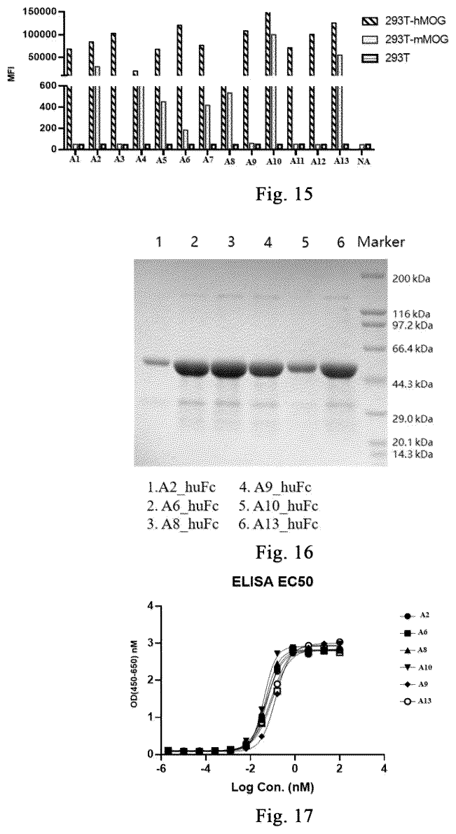

The results are shown in FIG. 15. Antibodies A1, A3, A9, All, and A12 bound to 293T-hMOG cells; antibodies A2, A4, A5, A6, A7, A8, A10, and A13 bound to 293T-hMOG cells and 293T-mMOG cells.

### Example 20. Determination of Antibody (Fab Form) Affinity Using Surface Plasmon Resonance (SPR)

The affinity of different antibodies against MOG was determined using biacoreT200. The specific steps were as follows: MOG_His was coated on the CM5 chip by amino coupling to about 150RU, and gradiently diluted antibodies were used as the mobile phase to pass through the channel of the coated antigen at a flow rate of 30 μl/min. The running buffer was HBS-N and the temperature was 25° C. The experimental data were analyzed using BIAevaluation3.2, and the kinetic curves were fitted using a 1:1 Langmuir model. The results are shown in Table 4.

TABLE 4
Antibody (Fab form) affinity

| Sample | ka (1/Ms) | kd (1/s) | KD (M) |
|---|---|---|---|
| A1 | 2.66E+05 | 1.93E−03 | 7.26E−09 |
| A2 | 1.80E+05 | 2.82E−03 | 1.56E−08 |
| A3 | 2.34E+05 | 1.39E−03 | 5.92E−09 |
| A4 | 2.15E+04 | 5.16E−04 | 2.40E−08 |
| A5 | 1.01E+05 | 2.78E−03 | 2.76E−08 |
| A6 | 8.45E+04 | 6.94E−04 | 8.22E−09 |
| A7 | 6.50E+04 | 1.66E−03 | 2.56E−08 |
| A8 | 1.28E+05 | 6.62E−04 | 5.18E−09 |
| A9 | 1.25E+05 | 2.87E−04 | 2.28E−09 |
| A10 | 7.48E+04 | 3.27E−03 | 4.37E−08 |
| A11 | 2.46E+05 | 4.32E−03 | 1.76E−08 |
| A12 | 1.51E+05 | 1.03E−03 | 6.83E−09 |
| A13 | 4.00E+04 | 1.05E−03 | 2.62E−08 |

### Example 21. Construction of Anti-MOG scFv_hFc Fusion Antibody and its Transient Expression and Purification in Eukaryotic Cells

Primers were designed for the VH and VL fragments of A2, A6, A8, A9, A10, and A13, respectively, and the corresponding scFv sequences were obtained and connected into appropriate eukaryotic expression vectors. 293F cells in the logarithmic growth phase were transiently transfected using 293fectin™ Transfection reagent (Invitrogen, 12347-019) or polyethyleneimine (PEI) (Sigma Aldrich, 408727). The culture supernatant was collected 5-7 days after transfection and subjected to affinity purification using Protein A. The obtained antibodies were quantitatively and qualitatively analyzed by SDS PAGE (FIG. 16).

### Example 22. The EC50 of Antibody (scFv_hFc Form) Binding to MOG was Determined by ELISA

The EC50 values of antibodies A2, A6, A8, A9, A10, and A13 binding to human MOG antigen was determined by standard ELISA. 2 μg/ml recombinant hMOG_his was coated on the immunoplates at 4° C. overnight. 2% MPBS was added for blocking at room temperature for 2 hours, and then washed three times with PBS. The tested antibodies after gradient dilution (A2, A6, A8, A9, A10, A13, a 5-fold gradient dilution was performed starting at 100 nM for 12 gradients) were added and incubated at room temperature for 1 hour. Then, washed three times with PBST (PBS containing 0.05% Tween-20) and three times with PBS; HRP-labeled anti-huFc tag antibody (1:4000, Sigma) was added and incubated at room temperature for 1 hour; washed three times with PBST and three times with PBS. TMBS substrate was added, the reaction was terminated after 10 minutes of color development, and the results were read on an ELISA reader (FIG. 17). The EC50 values are shown in Table 5.

TABLE 5
EC50 values of antibody (scFv_hFc form) binding to MOG

| antibody (scFv_hFc) | A2 | A6 | A8 | A9 | A10 | A13 |
|---|---|---|---|---|---|---|
| EC50(nM) | 0.052 | 0.056 | 0.047 | 0.135 | 0.040 | 0.09 |

### Example 23. The EC50 of Antibody (scFv_hFC Form) Binding to 293T-hMOG Cells and 293T-mMOG Cells was Determined by FACs

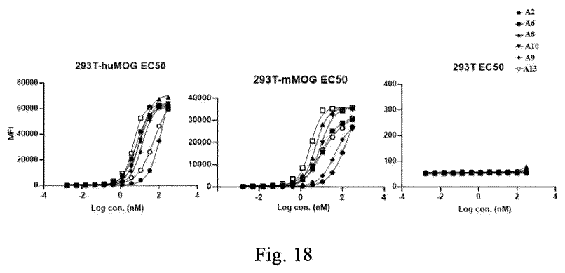

2×10⁵ 293T-hMOG or 293T-mMOG cells were added to 96-well plates, and the antibodies to be tested (A2, A6, A8, A9, A10, A13, 3-fold gradient dilution was performed starting at 600 nM for 11 gradients) were added and incubated at 4° C. for 45 minutes. Then, washed twice with PBS (1% FBS) and incubated with FITC-labeled anti-human IgG secondary antibody. After two washing steps, detection was performed using a BD FACSLyric instrument. The results are shown in FIG. 18, and the specific EC50 is shown in Table 6.

TABLE 6
EC50 values of antibody (scFv form) binding to cells

| | | |
|---|---|---|
| antibody | 293T-hMOG cells | 293T-mMOG cells |

| | | | | |
|---|---|---|---|---|
| A2 | 9.04 | nM | 7.62 | nM |
| A6 | 6.86 | nM | 12.56 | nM |
| A8 | 4.29 | nM | 6.21 | nM |
| A9 | 12.49 | nM | 130.5 | nM |
| A10 | 8.80 | nM | 3.62 | nM |
| A13 | 48.86 | nM | 2.35 | nM |

### Example 24: MOG-synE and MOG-synE-Del3 Regulate Gene Expression

The corresponding Jurkat responding cells of MOG-binding chimeric polypeptide MOG-synE or MOG-synE-del3 were constructed using MOG antibodies A1, A2, A3, A4, A5, A6, A7, A8, A9, A10, A11, A12, or A13 respectively according to the above examples, and mixed with the target cells at a ratio of 1:1. After 24 h of incubation, BFP was detected by flow cytometry. Target cells: 293T-hMOG, K562-hMOG, 293T-mMOG, or K562-mMOG. The results shows that after co-incubation with target cells, Jurkat responding cells expressing chimeric polypeptides targeting MOG all initiated BFP expression in the presence of MOG protein.

The embodiments of this application include the embodiment as any single embodiment or in combination with any other embodiment or part thereof. In addition, it should be understood that, after reading the above teachings of this application, those skilled in the art may make various changes or modifications to this application, and these equivalent forms also fall within the scope defined by the claims attached to this application.

### SEQUENCE INFORMATION

| | |
|---|---|
| 1 | gqdassagstrnkdptrrpeleagtngrssttspfvkpnpgsstdgnsaghsgnnilgsevalfa |
| 2 | ggacaagatgcaagttctgctggatcaaccaggaataaagatccaacaagacgtccagaactagaagctggtacaaatggaagaagttcgacaa |
| | caagtccctttgtaaaaccaaatccaggttctagcacagacggcaacagcgccggacattcggggaacaacatcctcggttccgaagtggcctta |
| | tttgca |
| 3 | gqdatrrpeleagtngrssttspfvkpnpgsstdgnsaghsgnnilgsevalfa |
| 4 | gqdassagstrnkdptrrpspfvkpnpgsstdgnsaghsgnnilgsevalfa |
| 5 | gqdassagstrnkdptrrpeleagtngrssttspsevalfa |
| 6 | gqdassagstrnkdptrrpsevalfa |
| 7 | asspclggnpcynqgtceptsespfyrclcpakfngllchgqdassagstrnkdptrrpeleagtngrssttspfvkpnpgsstdgnsaghsgn |
| | nilgsevalfa |
| 8 | dartcgslrclnggtcisgprsptclclgpftgpecqfpasspclggnpcynqgtceptsespfyrclcpakfngllchgqdassagstrnkdptrr |
| | peleagtngrssttspfvkpnpgsstdgnsaghsgnnilgsevalfa |
| 9 | vingckgkpcknggtcavasntargfickcpagfegatcendartcgslrclnggtcisgprsptclclgpftgpecqfpasspclggnpcynq |
| | gtceptsespfyrclcpakfngllchgqdassagstrnkdptrrpeleagtngrssttspfvkpnpgsstdgnsaghsgnnilgsevalfa |
| 10 | dvneclsnpcdargtqncvqrvndfhcecraghtgrrcesvingckgkpcknggtcavasntargfickcpagfegatcendartcgslrcln |
| | ggtcisgprsptclclgpftgpecqfpasspclggnpcynqgtceptsespfyrclcpakfngllchgqdassagstrnkdptrrpeleagtngrs |
| | sttspfvkpnpgsstdgnsaghsgnnilgsevalfa |
| 11 | lmyvaaaafvllffvgcgvllsrkrrr |
| 12 | llyllavavviilffillgvimakrkrkh |
| 13 | llpllvagavflliifilgvmvarrkreh |
| 14 | pilcspvvgvlllalgallvlqlirrrrreh |
| 15 | fmyvaaaafvllffvgcgvllsrkrrr |
| 16 | llyllavavviilfiillgvimakrkrkh |
| 17 | lpllvagavlllvilvlgvmvarrkreh |
| 18 | pvlcspvagvillalgallvlqlirrrrreh |
| 19 | lmyvaaaafvllffvgcgvllskrkrkh |
| 20 | lmyvaaaafvllffvgcgvllsrrkreh |
| 21 | lmyvaaaafvllffvgcgvllsrrrrreh |
| 22 | saligllviavaiatvivislvmlrkrq |
| 23 | rkrrr |
| 24 | krkrkh |
| 25 | rrkreh |
| 26 | rrrrreh |
| 27 | rkrq |
| 28 | mkllssieqacdicrlkklkcskekpkcakclknnwecryspktkrspltrahltevesrlerleqlfllifpredldmilkmdslqdikalltgl |
| | fvqdnvnkdavtdrlasvetdmpltlrqhrisatssseessnkgqrqltvsaaaggsggsggsdalddfdldmlgsdalddfdldmlgsdalddf |
| | dldmlgsdalddfdldmlgs |
| 29 | aagcttgatatcgaattcggagcactgtcctccgaacgteggagcactgtcctccgaacgteggagcactgtcctccgaacgtcggagcactgtc |
| | ctccgaacggagcatgtcctccgaacgteggagcactgtcctccgaacg |
| 30 | aagcttgatatcgaattcggagcactgtcctccgaacgtcggagcactgtcctccgaacgtcggagcactgtcctccgaacgtcggagcactgtc |
| | ctccgaacggagcatgtcctccgaacgtcggagcactgtcctccgaacgactagttaggcgtgtacggtgggaggcctatataagcagagctcgt |
| | ttagtgaaccgtcagatcgcctggagacgccatccacgctgttttgacctccatagaagacaccgggaccgatccagcctctcgacattcgttgga |
| | tc |
| 31 | accacgacgccagcgccgcgaccaccaacaccggcgcccaccategcgtcgcagcccctgtccctgcgcccagaggcgtgccggccagcg |
| | gcggggggcgcagtgcacacgagggggctggacttcgcctgtgat |
| 32 | atctacatctgggcgcccttggccgggacttgtggggtccttctcctgtcactggttatcaccctttactgc |
| 33 | ttttgggtgctggtggtggttggtggagtcctggcttgctatagcttgctagtaacagtggcctttattattttctgggtg |
| 34 | aggagtaagaggagcaggctcctgcacagtgactacatgaacatgactccccgccgccccgggccaacccgcaagcattaccagccctatgcc |
| | ccaccacgcgacttcgcagcctatcgctcc |
| 35 | aaacggggcagaaagaaactcctgtatatattcaaacaaccatttatgagaccagtacaaactactcaagaggaagatggctgtagctgccgattt |
| | ccagaagaagaagaaggaggatgtgaactg |
| 36 | agagtgaagttcagcaggagcgcagacgcccccgcgtaccagcagggccagaaccagctctataacgagctcaatctaggacgaagagagg |
| | agtacgatgttttggacaagagacgtggccgggaccctgagatggggggaaagccgcagagaaggaagaaccctcaggaaggcctgtacaat |
| | gaactgcagaaagataagatggcggaggcctacagtgagattgggatgaaaggcgagcgccggaggggcaaggggcacgatggcctttacc |
| | agggtctcagtacagccaccaaggacacctacgacgcccttcacatgcaggccctgccccctcgc |
| 37 | atggccttaccagtgaccgccttgctcctgccgctggccttgctgctccacgccgccaggccg |
| 38 | tttpaprpptpaptiasqplslrpeacrpaaggavhtrgldfacdfwvlvvvggvlacysllvtvafiifwvrskrsrllhsdymnmtprrpgptr |
| | khyqpyapprdfaayrsrvkfsrsadapayqqgqnqlynelnlgrreeydvldkrrgrdpemggkpqrrknpqeglynelqkdkmaeay |
| | seigmkgerrrgkghdglyqglstatkdtydalhmqalppr |
| 39 | tttpaprpptpaptiasqplslrpeacrpaaggavhtrgldfacdiyiwaplagtcgvlllslvitlyckrgrkkllyifkqpfmrpvqttqeedg |
| | cscrfpeeeeggcelrvkfsrsadapayqqgqnqlynelnlgrreeydvldkrrgrdpemggkpqrrknpqeglynelqkdkmaeayseigm |
| | kgerrrgkghdglyqglstatkdtydalhmqalppr |
| 40 | tttpaprpptpaptiasqplslrpeacrpaaggavhtrgldfacdfwvlvvvggvlacysllvtvafiifwvrskrsrllhsdymnmtprrpgptr |
| | khyqpyapprdfaayrskrgrkkllyifkqpfmrpvqttqeedgescrfpeeeeggcelrvkfsrsadapayqqgqnqlynelnlgrreeyd |
| | vldkrrgrdpemggkpqrrknpqeglynelqkdkmaeayseigmkgerrrgkghdglyqglstatkdtydalhmqalppr |
| 41 | MASLSRPSLPSCLCSFLLLLLLQVSSSYAGQFRVIGPRHPIRALVGDEVELPCRISPGKNATGM |
| | EVGWYRPPFSRVVHLYRNGKDQDGDQAPEYRGRTELLKDAIGEGKVTLRIRNVRFSDEGGF |
| | TCFFRDHSYQEEAAMELKVEDPFYWVSPGVLVLLAVLPVLLLQITVGLVFLCLQYRLRGKLR |
| | AEIENLHRTFDPHFLRVPCWKITLFVIVPVLGPLVALIICYNWLHRRLAGQFLEELRNPF |
| 42 | Mgwirgrrsrhswemsefhnynldlkksdfstrwqkqrcpvvkskcrenaspfffccfiavamgirfiimvaiwsavflnslfnqevqiplt |
| | esycgpcpknwicyknncyqffdesknwyesqascmsqnasllkvyskedqdllklvksyhwmglvhiptngswqwedgsilspnllti |
| | iemqkgdcalyassfkgyiencstpntyicmqrtv |
| 43 | EVQLLESGGGLVQPGGSLRLSCAASGFTFSSYAMSWVRQAPGKGLEWVSAISGSGGSTYYA |
| | DSVKGRFTISRDNSKNTLYLQMNSLRAEDTAVYYCAKEYFYAMDYWGQGTLVTVSSGGGG |
| | SGGGGSGGGGSDIQMTQSPSTLSASVGDRVTITCRASQSISSWLAWYQQKPGKAPKLLIYDA |
| | SSLESGVPSRFSGSGSGTEFTLTISSLQPDDFATYYCQQYTVYSPVTFGQGTKVEIKR |
| 44 | eqkliseedlevqlvqsgaevkkpgasvkvsckasgytfsdyemhwvrqapgqglewmgaihpgsgdtaynqrfkgrvtitadkststay |
| | melsslrsedtavyycarfysyaywgqgtlvtvsaggggsggggsggggsdivmtqtplslpvtpgepasiscrssqslvhsngntylqwyl |
| | qkpgqspqlliykvsnrfsgvpdrfsgsgsgtdftlkisrveaedvgvyycsqsiyvpytfgqgtkleikrgqdassagstrnkdptrrpeleag |
| | tngrssttspfvkpnpgsstdgnsaghsgnnilgsevalfalmyvaaaafvllffvgcgvllsrkrrrmkllssieqacdicrlkklkcskekpkc |
| | akclknnwecryspktkrspltrahltevesrlerleqlfllifpredldmilkmdslqdikalltglfvqdnvnkdavtdrlasvetdmpltlrqh |
| | risatssseessnkgqrqltvsaaaggsggsggsdalddfdldmlgsdalddfdldmlgsdalddfdldmlgsdalddfdldmlgs |
| 45 | eqkliseedlevqlvqsgaevkkpgasvkvsckasgytfsdyemhwvrqapgqglewmgaihpgsgdtaynqrfkgrvtitadkststay |
| | melsslrsedtavyycarfysyaywgqgtlvtvsaggggsggggsggggsdivmtqtplslpvtpgepasiscrssqslvhsngntylqwyl |
| | qkpgqspqlliykvsnrfsgvpdrfsgsgsgtdftlkisrveaedvgvyycsqsiyvpytfgqgtkleikrgqdassagstrnkdptrrpeleag |
| | tngrssttspsevalfalmyvaaaafvllffvgcgvllsrkrrrmkllssieqacdicrlkklkcskekpkcakclknnwecryspktkrspltrah |
| | ltevesrlerleqlfllifpredldmilkmdslqdikalltglfvqdnvnkdavtdrlasvetdmpltlrqhrisatssseessnkgqrqltvsaaa |
| | ggsggsggsdalddfdldmlgsdalddfdldmlgsdalddfdldmlgsdalddfdldmlgs |
| 46 | evqllesggglvqpggslrlscaasgftfssyamswvrqapgkglewvsaisgsggstyyadsvkgrftisrdnskntlylqmnslraedtav |
| | yycardwggiyyfdywgqgtlvtvssggggsggggsggggsdiqmtqspstlsasvgdrvtitcrasqsisswlawyqqkpgkapklliyd |
| | asslesgvpsrfsgsgsgteftltisslqpddfatyycqqkdtwsytfgqgtkveikrgqdassagstrnkdptrrpeleagtngrssttspfvkpn |
| | pgsstdgnsaghsgnnilgsevalfalmyvaaaafvllffvgcgvllsrkrrrmkllssieqacdicrlkklkcskekpkcakclknnwecrys |
| | pktkrspltrahltevesrlerleqlfllifpredldmilkmdslqdikalltglfvqdnvnkdavtdrlasvetdmpltlrqhrisatssseessn |
| | kgqrqltvsaaaggsggsggsdalddfdldmlgsdalddfdldmlgsdalddfdldmlgsdalddfdldmlgs |
| 47 | evqllesggglvqpggslrlscaasgftfssyamswvrqapgkglewvsaisgsggstyyadsvkgrftisrdnskntlylqmnslraedtav |
| | yycardwggiyyfdywgqgtlvtvssggggsggggsggggsdiqmtqspstlsasvgdrvtitcrasqsisswlawyqqkpgkapklliyd |
| | asslesgvpsrfsgsgsgteftltisslqpddfatyycqqkdtwsytfgqgtkveikrgqdassagstrnkdptrrpeleagtngrssttspseval |
| | falmyvaaaafvllffvgcgvllsrkrrrmkllssieqacdicrlkklkcskekpkcakclknnwecryspktkrspltrahltevesrlerleqlf |
| | llifpredldmilkmdslqdikalltglfvqdnvnkdavtdrlasvetdmpltlrqhrisatssseessnkgqrqltvsaaaggsggsggsdalddf |
| | dldmlgsdalddfdldmlgsdalddfdldmlgsdalddfdldmlgs |
| 48 | evqllesggglvqpggslrlscaasgftfssyamswvrqapgkglewvsaisgsggstyyadsvkgrftisrdnskntlylqmnslraedtav |
| | yycakddsfyggfdywgqgtlvtvssggggsggggsggggsdiqmtqspstlsasvgdrvtitcrasqsisswlawyqqkpgkapklliyd |
| | asslesgvpsrfsgsgsgteftltisslqpddfatyycqqyssykytfgqgtkveikrgqdassagstrnkdptrrpeleagtngrssttspfvkpn |
| | pgsstdgnsaghsgnnilgsevalfalmyvaaaafvllffvgcgvllsrkrrrmkllssieqacdicrlkklkcskekpkcakclknnwecrys |
| | pktkrspltrahltevesrlerleqlfllifpredldmilkmdslqdikalltglfvqdnvnkdavtdrlasvetdmpltlrqhrisatssseessn |
| | kgqrqltvsaaaggsggsggsdalddfdldmlgsdalddfdldmlgsdalddfdldmlgsdalddfdldmlgs |
| 49 | evqllesggglvqpggslrlscaasgftfssyamswvrqapgkglewvsaisgsggstyyadsvkgrftisrdnskntlylqmnslraedtav |
| | yycakddsfyggfdywgqgtlvtvssggggsggggsggggsdiqmtqspstlsasvgdrvtitcrasqsisswlawyqqkpgkapklliyd |
| | asslesgvpsrfsgsgsgteftltisslqpddfatyycqqyssykytfgqgtkveikrgqdassagstrnkdptrrpeleagtngrssttspseval |
| | falmyvaaaafvllffvgcgvllsrkrrrmkllssieqacdicrlkklkcskekpkcakclknnwecryspktkrspltrahltevesrlerleqlf |
| | llifpredldmilkmdslqdikalltglfvqdnvnkdavtdrlasvetdmpltlrqhrisatssseessnkgqrqltvsaaaggsggsggsdalddf |
| | dldmlgsdalddfdldmlgsdalddfdldmlgsdalddfdldmlgs |
| 50 | evqllesggglvqpggslrlscaasgftfssyamswvrqapgkglewvsaisgsggstyyadsvkgrftisrdnskntlylqmnslraedtav |
| | yycakyravtfdywgqgtlvtvssggggggggsggggsdiqmtqspstlsasvgdrvtitcrasqsisswlawyqqkpgkapklliydass |
| | lesgvpsrfsgsgsgteftltisslqpddfatyycqqfetqirwtfgqgtkveikrtttpaprpptpaptiasqplslrpeacrpaaggavhtrgld |
| | facdfwvlvvvggvlacysllvtvafiifwvrskrsrllhsdymnmtprrpgptrkhyqpyapprdfaayrsrvkfsrsadapayqqgqnqlyn |
| | elnlgrreeydvldkrrgrdpemggkpqrrknpqeglynelqkdkmaeayseigmkgerrrgkghdglyqglstatkdtydalhmqalpp |
| | r |
| 51 | mrvkfsrsadapayqqgqnqlynelnlgrreeydvldkrrgrdpemggkpqrrknpqeglynelqkdkmaeayseigmkgerrrgkgh |
| | dglyqglstatkdtydalhmqalpprkrgrkkllyifkqpfmrpvqttqeedgescrfpeeeeggcelgwirgrrsrhswemsefhnynldlk |
| | ksdfstrwqkqrcpvvkskcrenaspfffccfiavamgirfiimvaiwsavflnslfnqevqipltesycgpcpknwicyknncyqffdesk |
| | nwyesqascmsqnasllkvyskedqdllklvksyhwmglvhiptngswqwedgsilspnlltiiemqkgdcalyassfkgyiencstpnt |
| | yicmqrtv |
| 52 | mrvkfsrsadapayqqgqnqlynelnlgrreeydvldkrrgrdpemggkpqrrknpqeglynelqkdkmaeayseigmkgerrrgkgh |
| | dglyqglstatkdtydalhmqalpprgwirgrrsrhswemsefhnynldlkksdfstrwqkqrcpvvkskcrenaspfffccfiavamgirfi |
| | imvaiwsavflnslfnqevqipltesycgpcpknwicyknncyqffdesknwyesqascmsqnasllkvyskedqdllklvksyhwmgl |
| | vhiptngswqwedgsilspnlltiiemqkgdcalyassfkgyiencstpntyicmqrtv |
| 53 | mrvkfsrsadapayqqgqnqlynelnlgrreeydvldkrrgrdpemggkpqrrknpqeglynelqkdkmaeayseigmkgerrrgkgh |
| | dglyqglstatkdtydalhmqalpprgwirgrrsrhswemsefhnynldlkksdfstrwqkqrcpvvkskcrenaspfffccfiavamgirfi |
| | imvaiwsavflnslfnqevqipltesycgpcpknwicyknncyqffdesknwyesqascmsqnasllkvyskedqdllklvksyhwmgl |
| | vhiptngswqwedgsilspnlltiiemqkgdcalyassfkgyiencstpntyicmqrtvgsgatnfsllkqagdveenpgpmihlghilfllll |
| | pvaaaqttpgersslpafypgtsgscsgcgslslpllaglvaadavasllivgavflcarprrspaqedgkvyinmpgrg |
| 54 | iwsavflnslfnqevqipltesycgpcpknwicyknncyqffdesknwyesqascmsqnasllkvyskedqdllklvksyhwmglvhipt |
| | ngswqwedgsilspnlltiiemqkgdcalyassfkgyiencstpntyicmqrtvtttpaprpptpaptiasqplslrpeacrpaaggavhtrgld |
| | facdfwvlvvvggvlacysllvtvafiifwvrskrsrllhsdymnmtprrpgptrkhyqpyapprdfaayrsrvkfsrsadapayqqgqnql |
| | ynelnlgrreeydvldkrrgrdpemggkpqrrknpqeglynelqkdkmaeayseigmkgerrrgkghdglyqglstatkdtydalhmqal |
| | ppr |
| 55 | aagcttgatatcgaattcggagcactgtcctccgaacgtcggagcactgtcctccgaacgtcggagcactgtcctccgaacgtcggagcactgtc |
| | ctccgaacggagcatgtcctccgaacgtcggagcactgtcctccgaacgactagttaggcgtgtacggtgggaggcctatataagcagagctcgt |
| | ttagtgaaccgtcagatcgcctggagacgccatccacgctgttttgacctccatagaagacaccgggaccgatccagcctctcgacattcgttgatc |
| | atatgccaccatgagagtgaagttcagcaggagcgcagacgcccccgcgtaccagcagggccagaaccagctctataacgagctcaatctagg |
| | acgaagagaggagtacgatgttttggacaagagacgtggccgggaccctgagatggggggaaagccgcagagaaggaagaaccctcagga |
| | aggcctgtacaatgaactgcagaaagataagatggcggaggcctacagtgagattgggatgaaaggcgagcgccggaggggcaaggggcac |
| | gatggcctttaccagggtctcagtacagccaccaaggacacctacgacgcccttcacatgcaggccctgccccctcgcgggtggattcgtggtcg |
| | gaggtctcgacacagctgggagatgagtgaatttcataattataacttggatctgaagaagagtgatttttcaacacgatggcaaaagcaaagatgt |
| | ccagtagtcaaaagcaaatgtagagaaaatgcatctccattttttttctgctgcttcatcgctgtagccatgggaatccgtttcattattatggtag |
| | ctatatggagtgctgtattcctaaactcattattcaaccaagaagttcaaattcccttgaccgaaagttactgtggcccatgtcctaaaaactggat |
| | atgttacaaaaataactgctaccaattttttgatgagagtaaaaactggtatgagagccaggcttcttgtatgtctcaaaatgccagccttctgaaa |
| | gtatacagcaaagaggaccaggatttacttaaactggtgaagtcatatcattggatgggactagtacacattccaacaaatggatcttggcagtggg |
| | aagatggctccattctctcacccaacctactaacaataattgaaatgcagaagggagactgtgcactctatgcctcgagctttaaaggctatataga |
| | aaactgttcaactccaaatacatacatctgcatgcaaaggactgtgtaagctagcatcgatggctccggtgcccgtcagtgggcagagcgcacatcg |
| | cccacagtccccgagaagttggggggaggggtcggcaattgaaccggtgcctagagaaggtggcgcggggtaaactgggaaagtgatgtcgtgtact |
| | ggctccgcctttttcccgagggtgggggagaaccgtatataagtgcagtagtcgccgtgaacgttctttttcgcaacgggtttgccgccagaacaca |
| | ggtgtcgtgacgcggatccaggcctaagcttacgcgtcctagcgctaccggtcgccaccatggccttaccagtgaccgccttgctcctgccgctggc |
| | cttgctgctccacgccgccaggccggaggtgcaattgctggagtctgggggaggcttggtacagcctggggggtccctgagactctcctgtgcag |
| | cctccggattcacctttagcagttatgccatgagctgggtccgccaggctccagggaaggggctggagtgggtctcagctattagtggtagtggtg |
| | gtagcacatactacgcagactccgtgaagggccggttcaccatctccagagacaattccaagaacacgctgtatctgcagatgaacagcctgaga |
| | gccgaggacacggccgtatattactgtgcgaaagatgattctttctacggtggtttcgactactggggccaaggaaccctggtcaccgtctcgagt |
| | ggtggaggcggttcaggcggaggtggttctggcggtggcggatcggacatccagatgacccagtctccttccaccctgtctgcatctgtaggaga |
| | ccgtgtcaccatcacttgccgtgccagtcagagtattagtagctggttggcctggtatcagcagaaaccagggaaagcccctaagctcctgatctat |
| | gatgcctccagtttggaaagtggggtcccatcacgtttcagcggcagtggatccgggacagaattcactctcaccatcagcagcttgcagcctgat |
| | gattttgcaacttattactgccaacagtactcttcttacaaatacacgtttggccagggcaccaaagtcgagatcaagcgtggacaagatgcaagtt |
| | ctgctggatcaaccaggaataaagatccaacaagacgtccagaactagaagctggtacaaatggaagaagttcgacaacaagtccctttgtaaaac |
| | caaatccaggttctagcacagacggcaacagcgccggacattcggggaacaacatcctcggttccgaagtggccttatttgcactcatgtacgtg |
| | gcagcggccgccttcgtgctcctgttctttgtgggctgtggggtgctgctgtcccgcaagcgccggcggatgaagctgctgagcagcatcgagca |
| | ggcctgtgacatctgccggctgaagaaactgaagtgcagcaaagaaaagcccaagtgcgccaagtgcctgaagaacaactgggagtgccggt |
| | acagccccaagaccaagagaagccccctgaccagagcccacctgaccgaggtggaaagccggctggaaagactggaacagctgtttctgctg |
| | atcttcccacgcgaggacctggacatgatcctgaagatggacagcctgcaggacatcaaggccctgctgaccggcctgttcgtgcaggacaacg |
| | tgaacaaggacgccgtgaccgacagactggccagcgtggaaaccgacatgcccctgaccctgcggcagcacagaatcagcgccaccagcag |
| | cagcgaggaaagcagcaacaagggccagcggcagctgacagtgtctgctgctgcaggcggaagcggaggctctggcggatctgatgccctg |
| | gacgacttcgacctggatatgctgggcagcgacgccctggatgattttgatctggacatgctgggatctgacgctctggacgatttcgatctcgaca |
| | tgttgggatcagatgcactggatgactttgacctggacatgctcggatca |
| 56 | aagcttgatatcgaattcggagcactgtcctccgaacgtcggagcactgtcctccgaacgtcggagcactgtcctccgaacgtcggagcactgtc |
| | ctccgaacggagcatgtcctccgaacgtcggagcactgtcctccgaacgactagttaggcgtgtacggtgggaggcctatataagcagagctcgt |
| | ttagtgaaccgtcagatcgcctggagacgccatccacgctgttttgacctccatagaagacaccgggaccgatccagcctctcgacattcgttgga |
| | tcatattgccaccatgagagtgaagttcagcaggagcgcagacgcccccgcgtaccagcagggccagaaccagctctataacgagctcaatcta |
| | ggacgaagagaggagtacgatgttttggacaagagacgtggccgggaccctgagatggggggaaagccgcagagaaggaagaaccctcag |
| | gaaggcctgtacaatgaactgcagaaagataagatggcggaggcctacagtgagattgggatgaaaggcgagcgccggaggggcaaggggc |
| | acgatggcctttaccagggtctcagtacagccaccaaggacacctacgacgcccttcacatgcaggccctgccccctcgcaaacggggcagaa |
| | agaaactcctgtatatattcaaacaaccatttatgagaccagtacaaactactcaagaggaagatggctgtagctgccgatttccagaagaagaaga |
| | aggaggatgtgaactggggtggattcgtggtcggaggtctcgacacagctgggagatgagtgaatttcataattataacttggatctgaagaagag |
| | tgatttttcaacacgatggcaaaagcaaagatgtccagtagtcaaaagcaaatgtagagaaaatgcatctccattttttttctgctgcttcatcgct |
| | gtagccatgggaatccgtttcattattatggtagctatatggagtgctgtattcctaaactcattattcaaccaagaagttcaaattcccttgaccg |
| | aaagttactgtggcccatgtcctaaaaactggatatgttacaaaaataactgctaccaattttttgatgagagtaaaaactggtatgagagccaggc |
| | ttcttgtatgtctcaaaatgccagccttctgaaagtatacagcaaagaggaccaggatttacttaaactggtgaagtcatatcattggatgggacta |
| | gtacacattccaacaaatggatcttggcagtgggaagatggctccattctctcacccaacctactaacaataattgaaatgcagaagggagactgtg |
| | cactctatgcctcgagctttaaaggctatatagaaaactgttcaactccaaatacttacatctgcatgcaaaggactgtgtaagctagcatcgatgg |
| | ctccggtgcccgtcagtgggcagagcgcacatcgcccacagtccccgagaagttggggggaggggtcggcaattgaaccggtgcctagagaaggtgg |
| | cgcggggtaaactgggaaagtgatgtcgtgtactggctccgcctttttcccgagggtgggggagaaccgtatataagtgcagtagtcgccgtgaacg |
| | ttctttttcgcaacgggtttgccgccagaacacaggtgtcgtgacgcggatccaggcctaagcttacgcgtgccaccatggccttaccagtgaccgc |
| | cttgctcctgccgctggccttgctgctccacgccgccaggccggaggtgcaattgctggagtctgggggaggcttggtacagcctggggggtccctg |
| | agactctcctgtgcagcctccggattcacctttagcagttatgccatgagctgggtccgccaggctccagggaaggggctggagtgggtctcagct |
| | attagtggtagtggtggtagcacatactacgcagactccgtgaagggccggttcaccatctccagagacaattccaagaacacgctgtatctgcag |
| | atgaacagcctgagagccgaggacacggccgtatattactgtgcgaaagatgattctttctacggtggtttcgactactggggccaaggaaccctg |
| | gtcaccgtctcgagtggtggaggcggttcaggcggaggtggttctggcggtggcggatcggacatccagatgacccagtctccttccaccctgtc |
| | tgcatctgtaggagaccgtgtcaccatcacttgccgtgccagtcagagtattagtagctggttggcctggtatcagcagaaaccagggaaagcccc |
| | taagctcctgatctatgatgcctccagtttggaaagtggggtcccatcacgtttcagcggcagtggatccgggacagaattcactctcaccatcagc |
| | agcttgcagcctgatgattttgcaacttattactgccaacagtactcttcttacaaatacacgtttggccagggcaccaaagtcgagatcaagcgtg |
| | gacaagatgcaagttctgctggatcaaccaggaataaagatccaacaagacgtccagaactagaagctggtacaaatggaagaagttcgacaaca |
| | agtccctttgtaaaaccaaatccaggttctagcacagacggcaacagcgccggacattcggggaacaacatcctcggttccgaagtggccttattt |
| | gcactcatgtacgtggcagcggccgccttcgtgctcctgttctttgtgggctgtggggtgctgctgtcccgcaagcgccggcggatgaagctgctg |
| | agcagcatcgagcaggcctgtgacatctgccggctgaagaaactgaagtgcagcaaagaaaagcccaagtgcgccaagtgcctgaagaacaa |
| | ctgggagtgccggtacagccccaagaccaagagaagccccctgaccagagcccacctgaccgaggtggaaagccggctggaaagactgga |
| | acagctgtttctgctgatcttcccacgcgaggacctggacatgatcctgaagatggacagcctgcaggacatcaaggccctgctgaccggcctgtt |
| | cgtgcaggacaacgtgaacaaggacgccgtgaccgacagactggccagcgtggaaaccgacatgcccctgaccctgcggcagcacagaatc |
| | agcgccaccagcagcagcgaggaaagcagcaacaagggccagcggcagctgacagtgtctgctgctgcaggcggaagcggaggctctggc |
| | ggatctgatgccctggacgacttcgacctggatatgctgggcagcgacgccctggatgattttgatctggacatgctgggatctgacgctctggac |
| | gatttcgatctcgacatgttgggatcagatgcactggatgactttgacctggacatgctcggatca |
| 57 | aagcttgatatcgaattcggagcactgtcctccgaacgtcggagcactgtcctccgaacgtcggagcactgtcctccgaacgtcggagcactgtc |
| | ctccgaacggagcatgtcctccgaacgtcggagcactgtcctccgaacgactagttaggcgtgtacggtgggaggcctatataagcagagctcgt |
| | ttagtgaaccgtcagatcgcctggagacgccatccacgctgttttgacctccatagaagacaccgggaccgatccagcctctcgacattcgttgga |
| | tcatatgccaccatgagagtgaagttcagcaggagcgcagacgcccccgcgtaccagcagggccagaaccagctctataacgagctcaatcta |
| | ggacgaagagaggagtacgatgttttggacaagagacgtggccgggaccctgagatggggggaaagccgcagagaaggaagaaccctcag |
| | gaaggcctgtacaatgaactgcagaaagataagatggcggaggcctacagtgagattgggatgaaaggcgagcgccggaggggcaaggggc |
| | acgatggcctttaccagggtctcagtacagccaccaaggacacctacgacgcccttcacatgcaggccctgccccctcgcgggtggattcgtggt |
| | cggaggtctcgacacagctgggagatgagtgaatttcataattataacttggatctgaagaagagtgatttttcaacacgatggcaaaagcaaagat |
| | gtccagtagtcaaaagcaaatgtagagaaaatgcatctccattttttttctgctgcttcatcgctgtagccatgggaatccgtttcattattatggt |
| | agctatatggagtgctgtattcctaaactcattattcaaccaagaagttcaaattcccttgaccgaaagttactgtggcccatgtcctaaaaactgg |
| | atatgttacaaaaataactgctaccaattttttgatgagagtaaaaactggtatgagagccaggcttcttgtatgtctcaaaatgccagccttctga |
| | aagtatacagcaaagaggaccaggatttacttaaactggtgaagtcatatcattggatgggactagtacacattccaacaaatggatcttggcagtg |
| | ggaagatggctccattctctcacccaacctactaacaataattgaaatgcagaagggagactgtgcactctatgcctcgagctttaaaggctatata |
| | gaaaactgttcaactccaaatacatacatctgcatgcaaaggactgtgggaagcggagctactaacttcagcctgctgaagcaggctggagacgtgg |
| | aggagaaccctggacctatgatccatctgggtcacatcctcttcctgcttttgctcccagtggctgcagctcagacgactccaggagagagatcatc |
| | actccctgccttttaccctggcacttcaggctcttgttccggatgtgggtccctctctctgccgctcctggcaggcctcgtggctgctgatgcggtg |
| | gcatcgctgctcatcgtgggggcggtgttcctgtgcgcacgcccacgccgcagccccgcccaagaagatggcaaagtctacatcaacatgccaggca |
| | ggggctaagctagcatcgatggctccggtgcccgtcagtgggcagagcgcacatcgcccacagtccccgagaagttggggggaggggtcggcaattg |
| | aaccggtgcctagagaaggtggcgcggggtaaactgggaaagtgatgtcgtgtactggctccgcctttttcccgagggtgggggagaaccgtatat |
| | aagtgcagtagtcgccgtgaacgttctttttcgcaacgggtttgccgccagaacacaggtgtcgtgacgcggatccaggcctaagcttacgcgtcc |
| | tagcgctaccggtcgccaccatggccttaccagtgaccgccttgctcctgccgctggccttgctgctccacgccgccaggccggaggtgcaattg |
| | ctggagtctgggggaggcttggtacagcctggggggtccctgagactctcctgtgcagcctccggattcacctttagcagttatgccatgagctgg |
| | gtccgccaggctccagggaaggggctggagtgggtctcagctattagtggtagtggtggtagcacatactacgcagactccgtgaagggccggt |
| | tcaccatctccagagacaattccaagaacacgctgtatctgcagatgaacagcctgagagccgaggacacggccgtatattactgtgcgaaagat |
| | gattctttctacggtggtttcgactactggggccaaggaaccctggtcaccgtctcgagtggtggaggcggttcaggcggaggtggttctggcggt |
| | ggcggatcggacatccagatgacccagtctccttccaccctgtctgcatctgtaggagaccgtgtcaccatcacttgccgtgccagtcagagtatta |
| | gtagctggttggcctggtatcagcagaaaccagggaaagcccctaagctcctgatctatgatgcctccagtttggaaagtggggtcccatcacgttt |
| | cagcggcagtggatccgggacagaattcactctcaccatcagcagcttgcagcctgatgattttgcaacttattactgccaacagtactcttcttac |
| | aaatacacgtttggccagggcaccaaagtcgagatcaagcgtggacaagatgcaagttctgctggatcaaccaggaataaagatccaacaagacg |
| | tccagaactagaagctggtacaaatggaagaagttcgacaacaagtccctttgtaaaaccaaatccaggttctagcacagacggcaacagcgcc |
| | ggacattcggggaacaacatcctcggttccgaagtggccttatttgcactcatgtacgtggcagcggccgccttcgtgctcctgttctttgtgggct |
| | gtggggtgctgctgtcccgcaagcgccggcggatgaagctgctgagcagcatcgagcaggcctgtgacatctgccggctgaagaaactgaagtg |
| | cagcaaagaaaagcccaagtgcgccaagtgcctgaagaacaactgggagtgccggtacagccccaagaccaagagaagccccctgaccaga |
| | gcccacctgaccgaggtggaaagccggctggaaagactggaacagctgtttctgctgatcttcccacgcgaggacctggacatgatcctgaaga |
| | tggacagcctgcaggacatcaaggccctgctgaccggcctgttcgtgcaggacaacgtgaacaaggacgccgtgaccgacagactggccagc |
| | gtggaaaccgacatgcccctgaccctgcggcagcacagaatcagcgccaccagcagcagcgaggaaagcagcaacaagggccagcggca |
| | gctgacagtgtctgctgctgcaggcggaagcggaggctctggcggatctgatgccctggacgacttcgacctggatatgctgggcagcgacgc |
| | cctggatgattttgatctggacatgctgggatctgacgctctggacgatttcgatctcgacatgttgggatcagatgcactggatgactttgacctg |
| | gacatgctcggatca |
| 58 | aagcttgatatcgaattcggagcactgtcctccgaacgtcggagcactgtcctccgaacgtcggagcactgtcctccgaacgtcggagcactgtc |
| | ctccgaacggagcatgtcctccgaacgtcggagcactgtcctccgaacgactagttaggcgtgtacggtgggaggcctatataagcagagctcgt |
| | ttagtgaaccgtcagatcgcctggagacgccatccacgctgttttgacctccatagaagacaccgggaccgatccagcctctcgacattcgttgga |
| | tcatggccttaccagtgaccgccttgctcctgccgctggccttgctgctccacgccgccaggccggaggtgcaattgctggagtctgggggaggc |
| | ttggtacagcctggggggtccctgagactctcctgtgcagcctccggattcacctttagcagttatgccatgagctgggtccgccaggctccaggg |
| | aaggggctggagtgggtctcagctattagtggtagtggtggtagcacatactacgcagactccgtgaagggccggttcaccatctccagagacaa |
| | ttccaagaacacgctgtatctgcagatgaacagcctgagagccgaggacacggccgtatattactgtgcgaaataccgtgcagttactttcgacta |
| | ctggggccaaggaaccctggtcaccgtctcgagtggtggaggcggttcaggcggaggtggttctggcggtggcggatcggacatccagatgac |
| | ccagtctccttccaccctgtctgcatctgtaggagaccgtgtcaccatcacttgccgtgccagtcagagtattagtagctggttggcctggtatcag |
| | cagaaaccagggaaagcccctaagctcctgatctatgatgcctccagtttggaaagtggggtcccatcacgtttcagcggcagtggatccgggaca |
| | gaattcactctcaccatcagcagcttgcagcctgatgattttgcaacttattactgccaacagttcgaaacccagatcagatggacgtttggccagg |
| | gcaccaaagtcgagatcaagcgtaccacgacgccagcgccgcgaccaccaacaccggcgcccaccatcgcgtcgcagcccctgtccctgcgcc |
| | cagaggcgtgccggccagcggcggggggcgcagtgcacacgagggggctggacttcgcctgtgatttttgggtgctggtggtggttggtggag |
| | tcctggcttgctatagcttgctagtaacagtggcctttattattttctgggtgaggagtaagaggagcaggctcctgcacagtgactacatgaacat |
| | gactccccgccgccccgggccaacccgcaagcattaccagccctatgccccaccacgcgacttcgcagcctatcgctccagagtgaagttcagca |
| | ggagcgcagacgcccccgcgtaccagcagggccagaaccagctctataacgagctcaatctaggacgaagagaggagtacgatgttttggac |
| | aagagacgtggccgggaccctgagatggggggaaagccgcagagaaggaagaaccctcaggaaggcctgtacaatgaactgcagaaagat |
| | aagatggcggaggcctacagtgagattgggatgaaaggcgagcgccggaggggcaaggggcacgatggcctttaccagggtctcagtacag |
| | ccaccaaggacacctacgacgcccttcacatgcaggccctgccccctcgctagtaagctagccgatggctccggtgcccgtcagtgggcagag |
| | cgcacatcgcccacagtccccgagaagttggggggaggggtcggcaattgaaccggtgcctagagaaggtggcgcggggtaaactgggaaa |
| | gtgatgtcgtgtactggctccgcctttttcccgaggggggggagaaccgtatataagtgcagtagtcgccgtgaacgttctttttcgcaacgggttt |
| | gccgccagaacacaggtgtcgtgacgcggatccaggcctaagcttacgcgtcctagcgctaccggtcgccaccatggcgctccctgtcaccgc |
| | actgcttcttccgctggcactgctgctgcacgctgcacggcctgaggtgcaattgctggagtctgggggaggcttggtacagcctggggggtccc |
| | tgagactctcctgtgcagcctccggattcacctttagcagttatgccatgagctgggtccgccaggctccagggaaggggctggagtgggtctca |
| | gctattagtggtagtggtggtagcacatactacgcagactccgtgaagggccggttcaccatctccagagacaattccaagaacacgctgtatctg |
| | cagatgaacagcctgagagccgaggacacggccgtatattactgtgcgcgtgattggggtggtatctactacttcgactactggggccaaggaac |
| | cctggtcaccgtctcgagtggtggaggcggttcaggcggaggtggttctggcggtggcggatcggacatccagatgacccagtctccttccacc |
| | ctgtctgcatctgtaggagaccgtgtcaccatcacttgccgtgccagtcagagtattagtagctggttggcctggtatcagcagaaaccagggaaa |
| | gcccctaagctcctgatctatgatgcctccagtttggaaagtggggtcccatcacgtttcagcggcagtggatccgggacagaattcactctcacca |
| | tcagcagcttgcagcctgatgattttgcaacttattactgccaacagaaagatacctggtcttacacgtttggccagggcaccaaagtcgagatcaa |
| | gcgtggacaagatgcaagttctgctggatcaaccaggaataaagatccaacaagacgtccagaactagaagctggtacaaatggaagaagttcg |
| | acaacaagtccctttgtaaaaccaaatccaggttctagcacagacggcaacagcgccggacattcggggaacaacatcctcggttccgaagtgg |
| | ccttatttgcactcatgtacgtggcagcggccgccttcgtgctcctgttctttgtgggctgtggggtgctgctgtcccgcaagcgccggcggatgaa |
| | gctgctgagcagcatcgagcaggcctgtgacatctgccggctgaagaaactgaagtgcagcaaagaaaagcccaagtgcgccaagtgcctga |
| | agaacaactgggagtgccggtacagccccaagaccaagagaagccccctgaccagagcccacctgaccgaggtggaaagccggctggaaa |
| | gactggaacagctgtttctgctgatcttcccacgcgaggacctggacatgatcctgaagatggacagcctgcaggacatcaaggccctgctgacc |
| | ggcctgttcgtgcaggacaacgtgaacaaggacgccgtgaccgacagactggccagcgtggaaaccgacatgcccctgaccctgcggcagca |
| | cagaatcagcgccaccagcagcagcgaggaaagcagcaacaagggccagcggcagctgacagtgtctgctgctgcaggcggaagcggagg |
| | ctctggcggatctgatgccctggacgacttcgacctggatatgctgggcagcgacgccctggatgattttgatctggacatgctgggatctgacgct |
| | ctggacgatttcgatctcgacatgttgggatcagatgcactggatgactttgacctggacatgctcggatca |
| 59 | aagcttgatatcgaattcggagcactgtcctccgaacgtcggagcactgtcctccgaacgtcggagcactgtcctccgaacgtcggagcactgtc |
| | ctccgaacggagcatgtcctccgaacgtcggagcactgtcctccgaacgactagttaggcgtgtacggtgggaggcctatataagcagagctcgt |
| | ttagtgaaccgtcagatcgcctggagacgccatccacgctgttttgacctccatagaagacaccgggaccgatccagcctctcgacattcgttgga |
| | tcatggccttaccagtgaccgccttgctcctgccgctggccttgctgctccacgccgccaggccggaggtgcaattgctggagtctgggggaggc |
| | ttggtacagcctggggggtccctgagactctcctgtgcagcctccggattcacctttagcagttatgccatgagctgggtccgccaggctccaggg |
| | aaggggctggagtgggtctcagctattagtggtagtggtggtagcacatactacgcagactccgtgaagggccggttcaccatctccagagacaa |
| | ttccaagaacacgctgtatctgcagatgaacagcctgagagccgaggacacggccgtatattactgtgcgaaataccgtgcagttactttcgacta |
| | ctggggccaaggaaccctggtcaccgtctcgagtggtggaggcggttcaggcggaggtggttctggcggtggcggatcggacatccagatgac |
| | ccagtctccttccaccctgtctgcatctgtaggagaccgtgtcaccatcacttgccgtgccagtcagagtattagtagctggttggcctggtatcag |
| | cagaaaccagggaaagcccctaagctcctgatctatgatgcctccagtttggaaagtggggtcccatcacgtttcagcggcagtggatccgggaca |
| | gaattcactctcaccatcagcagcttgcagcctgatgattttgcaacttattactgccaacagttcgaaacccagatcagatggacgtttggccagg |
| | gcaccaaagtcgagatcaagcgtaccacgacgccagcgccgcgaccaccaacaccggcgcccaccatcgcgtcgcagcccctgtccctgcgcc |
| | cagaggcgtgccggccagcggcggggggcgcagtgcacacgagggggctggacttcgcctgtgatttttgggtgctggtggtggttggtggag |
| | tcctggcttgctatagcttgctagtaacagtggcctttattattttctgggtgaggagtaagaggagcaggctcctgcacagtgactacatgaacat |
| | gactccccgccgccccgggccaacccgcaagcattaccagccctatgccccaccacgcgacttcgcagcctatcgctccagagtgaagttcagca |
| | ggagcgcagacgcccccgcgtaccagcagggccagaaccagctctataacgagctcaatctaggacgaagagaggagtacgatgttttggac |
| | aagagacgtggccgggaccctgagatggggggaaagccgcagagaaggaagaaccctcaggaaggcctgtacaatgaactgcagaaagat |
| | aagatggcggaggcctacagtgagattgggatgaaaggcgagcgccggaggggcaaggggcacgatggcctttaccagggtctcagtacag |
| | ccaccaaggacacctacgacgcccttcacatgcaggccctgccccctcgctagtaagctagccgatggctccggtgcccgtcagtgggcagag |
| | cgcacatcgcccacagtccccgagaagttggggggaggggtcggcaattgaaccggtgcctagagaaggtggcgcggggtaaactgggaaa |
| | gtgatgtcgtgtactggctccgcctttttcccgaggggggggagaaccgtatataagtgcagtagtcgccgtgaacgttctttttcgcaacgggttt |
| | gccgccagaacacaggtgtcgtgacgcggatccaggcctaagcttacgcgtcctagcgctaccggtcgccaccatggcgctccctgtcaccgc |
| | actgcttcttccgctggcactgctgctgcacgctgcacggcctgaggtgcaattgctggagtctgggggaggcttggtacagcctggggggtccc |
| | tgagactctcctgtgcagcctccggattcacctttagcagttatgccatgagctgggtccgccaggctccagggaaggggctggagtgggtctca |
| | gctattagtggtagtggtggtagcacatactacgcagactccgtgaagggccggttcaccatctccagagacaattccaagaacacgctgtatctg |
| | cagatgaacagcctgagagccgaggacacggccgtatattactgtgcgcgtgattggggtggtatctactacttcgactactggggccaaggaac |
| | cctggtcaccgtctcgagtggtggaggcggttcaggcggaggtggttctggcggtggcggatcggacatccagatgacccagtctccttccacc |
| | ctgtctgcatctgtaggagaccgtgtcaccatcacttgccgtgccagtcagagtattagtagctggttggcctggtatcagcagaaaccagggaaa |
| | gcccctaagctcctgatctatgatgcctccagtttggaaagtggggtcccatcacgtttcagcggcagtggatccgggacagaattcactctcacca |
| | tcagcagcttgcagcctgatgattttgcaacttattactgccaacagaaagatacctggtcttacacgtttggccagggcaccaaagtcgagatcaa |
| | gcgtggacaagatgcaagttctgctggatcaaccaggaataaagatccaacaagacgtccagaactagaagctggtacaaatggaagaagttcg |
| | acaacaagtccctccgaagtggccttatttgcactcatgtacgtggcagcggccgccttcgtgctcctgttctttgtgggctgtggggtgctgctgt |
| | cccgcaagcgccggcggatgaagctgctgagcagcatcgagcaggcctgtgacatctgccggctgaagaaactgaagtgcagcaaagaaaag |
| | cccaagtgcgccaagtgcctgaagaacaactgggagtgccggtacagccccaagaccaagagaagccccctgaccagagcccacctgaccg |
| | aggtggaaagccggctggaaagactggaacagctgtttctgctgatcttcccacgcgaggacctggacatgatcctgaagatggacagcctgca |
| | ggacatcaaggccctgctgaccggcctgttcgtgcaggacaacgtgaacaaggacgccgtgaccgacagactggccagcgtggaaaccgaca |
| | tgcccctgaccctgcggcagcacagaatcagcgccaccagcagcagcgaggaaagcagcaacaagggccagcggcagctgacagtgtctgc |
| | tgctgcaggcggaagcggaggctctggcggatctgatgccctggacgacttcgacctggatatgctgggcagcgacgccctggatgattttgatc |
| | tggacatgctgggatctgacgctctggacgatttcgatctcgacatgttgggatcagatgcactggatgactttgacctggacatgctcggatca |
| 60 | gqdassagstrnkdptrrpeleagtngrssttspfvkpnpgsstdgnsaghsgnnilgsevalfalmyvaaaafvllffvgcgvllsrkrrr |
| 61 | gqdatrrpeleagtngrssttspfvkpnpgsstdgnsaghsgnnilgsevalfalmyvaaaafvllffvgcgvllsrkrrr |
| 62 | gqdassagstrnkdptrrpspfvkpnpgsstdgnsaghsgnnilgsevalfalmyvaaaafvllffvgcgvllsrkrrr |
| 63 | gqdassagstrnkdptrrpeleagtngrssttspsevalfalmyvaaaafvllffvgcgvllsrkrrr |
| 64 | gqdassagstrnkdptrrpsevalfalmyvaaaafvllffvgcgvllsrkrrr |
| 65 | evigfgrscwsrgtpfphgsswvedcnscrcldgrrdcskvwcgwkpcllagqpealsaqcplgqrclekapgqclrppceawgecgaee |
| | ppstpclprsghldnncarltlhfnrdhvpqgttvgaicsgirslpatravardrllvllcdrassgasavevavsfspardlpdssliqgaahai |
| | vaaitqrgnsslllavtevkvetvvtggsstg |
| 66 | evigfgrscwsrgtpfphgsswvedcnscrcldgrrdcskvwcgwkpcllagqpealsaqcplgqrclekapgqclrppceawgecgaee |
| | ppstpclprsghldnncarltlhfnrdhvpqgttvgaicsgirslpatravardrllvllcdrassgasavevavsfspardlpdssliqgaahai |
| | vaaitqrgnsslllavtevkvetvvtggsstglmyvaaaafvllffvgcgvllsrkrrr |
| 67 | gqdassagstrnkdptrrpeleagtngrssttspfvkpnpgsstdgnsaghsgnnilgsevalfasaligllviavaiatvivislvmlrkrq |
| 68 | evqllesggglvqpggslrlscaasgftfssyamswvrqapgkglewvsaisgsggstyyadsvkgrftisrdnskntlylqmnslraedtav |
| | yycardwggiyyfdywgqgtlvtvssggggsggggsggggsdiqmtqspstlsasvgdrvtitcrasqsisswlawyqqkpgkapklliyd |
| | asslesgvpsrfsgsgsgteftltisslqpddfatyycqqkdtwsytfgqgtkveikr |
| 69 | evqlvqsgaevkkpgasvkvsckasgytfsdyemhwvrqapgqglewmgaihpgsgdtaynqrfkgrvtitadkststaymelsslrsed |
| | tavyycarfysyaywgqgtlvtvsaggggsggggsggggsdivmtqtplslpvtpgepasiscrssqslvhsngntylqwylqkpgqspqll |
| | iykvsnrfsgvpdrfsgsgsgtdftlkisrveaedvgvyycsqsiyvpytfgqgtkleikr |
| 70 | evqllesggglvqpggslrlscaasgftfssyamswvrqapgkglewvsaisgsggstyyadsvkgrftisrdnskntlylqmnslraedtav |
| | yycakyravtfdywgqgtlvtvssggggsggggsggggsdiqmtqspstlsasvgdrvtitcrasqsisswlawyqqkpgkapklliydass |
| | lesgvpsrfsgsgsgteftltisslqpddfatyycqqfetqirwtfgqgtkveikr |
| 71 | evqllesggglvqpggslrlscaasgftfssyamswvrqapgkglewvsaisgsggstyyadsvkgrftisrdnskntlylqmnslraedtav |
| | yycakddsfyggfdywgqgtlvtvssggggsggggsggggsdiqmtqspstlsasvgdrvtitcrasqsisswlawyqqkpgkapklliyd |
| | asslesgvpsrfsgsgsgteftltisslqpddfatyycqqyssykytfgqgtkveikr |
| 72 | diqmtqspsslsasvgdrvtitchasqdinvnigwlqqkpgkafkgliyhgknledgvpsrfsgsgsgtdftltisslqpedfatyycnqyeni |
| | pltfgqgtkveikrggggsggggsggggsdvqlvesggglvqpggslrlscavsgysitsdyawnwirqapgkglewlgyisyrgrtqynps |
| | lksrisitrdnskntfflqlnslraedtavyycarmgknwdywgqgtlvtvss |
| 73 | qmqlvqsgaevkkpgasvkvsckasgytftsyymhwvrqapgqglewmgiinpsggstsyaqkfqgrvtmtrdtststvymelsslrse |
| | dtavyycassrsgttvvnhdafdiwgkgttvtvssggggsggggsggggsdiqltqspsslsasvgdrvtitcrasqvisralawyqqtpgkpp |
| | klliydasnlqsgvpsrfsgsgsgtdftltisrlqpedfatyycqqfnsypltfgggtkleikr |
| 74 | qvqlvqsgaevkkpgssvkvsckasggtfssyaiswvrqapgqglewmggiipifgtanyaqkfqgrvtitadkststaymelsslrsedta |
| | vyycardaadrdywgqgttvtvssggggsggggsggggsdivmtqsplslpvtpgepasiscrssqsllhpngfnhlywylqkpgqspqlli |
| | yvggnrasgvpdrfsgsgsgtdftlkisrveaedvgvyycqqrnnknrtfgqgtkveikr |
| 75 | qvqlqesgpglikpsqtlsltctvsggsissgynwhwirqppgkglewigyihytgstnynpalrsrvtisvdtsknqfslklssvtaadtaiyyc |
| | ariyngnsfpywgqgttvtvssggggsggggsggggsdivmtqspdslavslgeratinckssqslfnsgnqknyltwyqqkpgqppklli |
| | ywastresgvpdrfsgsgsgtdftltisslqaedvavyycqnaysfpytfgggtkleikr |
| 76 | evqllesggglvqpggslrlscaasgftfssyamswvrqapgkglewvsaisgsggstyyadsvkgrftisrdnskntlylqmnslraedtav |
| | yycargwpspvtfdywgqgtlvtvssggggsggggsggggseivltqspgtlslspgeratlscrasqsvsssylawyqqkpgqaprlliyga |
| | ssratgipdrfsgsgsgtdftltisrlepedfavyycqqykshpitfgqgtkveikr |
| 77 | QVQLVQSGAEVKKPGSSVKVSCKASGGTFSSYAISWVRQAPGQGLEWMGGIIPIFGTANYAQ |
| | KFQGRVTITADKSTSTAYMELSSLRSEDTAVYYCARAWYFGYDFDYWGQGTTVTVSSGGGG |
| | SGGGGSGGGGSDIQMTQSPSTLSASVGDRVTITCRASQSISSWLAWYQQKPGKAPKLLIYDA |
| | SSLESGVPSRFSGSGSGTEFTLTISSLQPDDFATYYCQQTAIYSTFGQGTKVEIKR |
| 78 | evqllesggglvqpggslrlscaasgftfggnamswvrqapgkglewvsaisgnggstfyadsvkgrftisrdnskntlylqmnslraedtav |
| | yycakvrpfwgtfdywgqgtlvtvssggggsggggsggggseivltqspgtlslspgeratlscrasqsvsssylawyqqkpgqaprlliyga |
| | ssratgipdrfsgsgsgtdftltisrlepedfavyycqqyfnppeytfgqgtkveikr |
| 79 | evqlvqsgaevkkpgasvkvsckasgytfnsyymhwvrqapgqglewmgifnpgggstsyaqkfqgrvtmtrdtststvymelsslrse |
| | dtavyycargttsdafdiwgqgtmvtvssggggsggggsggggsdiqmtqspsslsasvgdrvtitcrasqsissylnwyqqkpgkapklli |
| | dvasslqsgvpsrfsgsgsgtdftltisslqpedfatyycqqsystpltfgggtkveikr |
| 80 | qvqlvqsgaevkkpgasvkvsckasgytftsywmnwvrqapgqglewmgridpydsethyaqklqgrvtmttdtststaymelrslrsd |
| | dtavyycarggydfdvgtlywffdvwgqgttvtvssggggsggggsggggsdiqmtqspsslsasvgdrvtitcraseniysylawyqqkp |
| | gkapklliynaktlaegvpsrfsgsgsgtdftltisslqpedfatyycqhhygtprtfgggtkveik |
| 81 | EVQLLESGGGLVQPGGSLRLSCAASGFTFSSYAMSWVRQAPGKGLEWVSAISGSGGSTYYA |
| | DSVKGRFTISRDNSKNTLYLQMNSLRAEDTAVYYCARSYNWYEWYWFDYWGQGTLVTVSS |
| | GGGGGGGGSGGGGSDIQMTQSPSTLSASVGDRVTITCRASQSISSWLAWYQQKPGKAPKLL |
| | IYDASSLESGVPSRFSGSGSGTEFTLTISSLQPDDFATYYCQQYNSFSITFGQGTKVEIKR |
| 82 | EVQLLESGGGLVQPGGSLRLSCAASGFTFSSYAMSWVRQAPGKGLEWVSAISGSGGSTYYA |
| | DSVKGRFTISRDNSKNTLYLQMNSLRAEDTAVYYCARPSLADWGDMDYWGQGTLVTVSSG |
| | GGGSGGGGSGGGGSDIQMTQSPSTLSASVGDRVTITCRASQSISSWLAWYQQKPGKAPKLLI |
| | YDASSLESGVPSRFSGSGSGTEFTLTISSLQPDDFATYYCQQYDQYWTFGQGTKVEIKR |
| 83 | EVQLLESGGGLVQPGGSLRLSCAASGFTFSSYAMSWVRQAPGKGLEWVSAISGSGGSTYYA |
| | DSVKGRFTISRDNSKNTLYLQMNSLRAEDTAVYYCAKDWVYFGVGSFDYWGQGTLVTVSS |
| | GGGGSGGGGSGGGGSDIQMTQSPSTLSASVGDRVTITCRASQSISSWLAWYQQKPGKAPKLL |
| | IYDASSLESGVPSRFSGSGSGTEFTLTISSLQPDDFATYYCQQTHSYSSTTFGQGTKVEIKR |
| 84 | RASQSISSWLA |
| 85 | DASSLES |
| 86 | QQYDSYYT |
| 87 | QQTAIYST |
| 88 | QQYDSPLLT |
| 89 | QQYHEYSPT |
| 90 | QQYSMDHLT |
| 91 | QQYNSFSIT |
| 92 | QQSDTYPRT |
| 93 | QQKDTWSYT |
| 94 | QQYDQYWT |
| 95 | QQTHSYSSTT |
| 96 | QQVYIYST |
| 97 | QQYGIPWT |
| 98 | QQYTVYSPVT |
| 99 | SYAIS |
| 100 | SYAMS |
| 101 | GIIPIFGTANYAQKFQG |
| 102 | AISGSGGSTYYADSVKG |
| 103 | GYFSEEAMDY |
| 104 | AWYFGYDFDY |
| 105 | GWYFFDAGFDY |
| 106 | EWYWPDNPFDY |
| 107 | APYSPTYYPFDY |
| 108 | SYNWYEWYWFDY |
| 109 | DWAGIYYLDY |
| 110 | DWGGIYYFDY |
| 111 | PSLADWGDMDY |
| 112 | DWVYFGVGSFDY |
| 113 | DYWLDSDYFDY |
| 114 | DYGTISAFDY |
| 115 | EYFYAMDY |
| 116 | DIQMTQSPSTLSASVGDRVTITCRASQSISSWLAWYQQKPGKAPKLLIYDASSLESGVPSRFS |
| | GSGSGTEFTLTISSLQPDDFATYYCQQYDSYYTFGQGTKVEIKR |
| 117 | DIQMTQSPSTLSASVGDRVTITCRASQSISSWLAWYQQKPGKAPKLLIYDASSLESGVPSRFS |
| | GSGSGTEFTLTISSLQPDDFATYYCQQTAIYSTFGQGTKVEIKR |
| 118 | DIQMTQSPSTLSASVGDRVTITCRASQSISSWLAWYQQKPGKAPKLLIYDASSLESGVPSRFS |
| | GSGSGTEFTLTISSLQPDDFATYYCQQYDSPLLTFGQGTKVEIKR |
| 119 | DIQMTQSPSTLSASVGDRVTITCRASQSISSWLAWYQQKPGKAPKLLIYDASSLESGVPSRFS |
| | GSGSGTEFTLTISSLQPDDFATYYCQQYHEYSPTFGQGTKVEIKR |
| 120 | DIQMTQSPSTLSASVGDRVTITCRASQSISSWLAWYQQKPGKAPKLLIYDASSLESGVPSRFS |
| | GSGSGTEFTLTISSLQPDDFATYYCQQYSMDHLTFGQGTKVEIKR |
| 121 | DIQMTQSPSTLSASVGDRVTITCRASQSISSWLAWYQQKPGKAPKLLIYDASSLESGVPSRFS |
| | GSGSGTEFTLTISSLQPDDFATYYCQQYNSFSITFGQGTKVEIKR |
| 122 | DIQMTQSPSTLSASVGDRVTITCRASQSISSWLAWYQQKPGKAPKLLIYDASSLESGVPSRFS |
| | GSGSGTEFTLTISSLQPDDFATYYCQQSDTYPRTFGQGTKVEIKR |
| 123 | DIQMTQSPSTLSASVGDRVTITCRASQSISSWLAWYQQKPGKAPKLLIYDASSLESGVPSRFS |
| | GSGSGTEFTLTISSLQPDDFATYYCQQKDTWSYTFGQGTKVEIKR |
| 124 | DIQMTQSPSTLSASVGDRVTITCRASQSISSWLAWYQQKPGKAPKLLIYDASSLESGVPSRFS |
| | GSGSGTEFTLTISSLQPDDFATYYCQQYDQYWTFGQGTKVEIKR |
| 125 | DIQMTQSPSTLSASVGDRVTITCRASQSISSWLAWYQQKPGKAPKLLIYDASSLESGVPSRFS |
| | GSGSGTEFTLTISSLQPDDFATYYCQQTHSYSSTTFGQGTKVEIKR |
| 126 | DIQMTQSPSTLSASVGDRVTITCRASQSISSWLAWYQQKPGKAPKLLIYDASSLESGVPSRFS |
| | GSGSGTEFTLTISSLQPDDFATYYCQQVYIYSTFGQGTKVEIKR |
| 127 | DIQMTQSPSTLSASVGDRVTITCRASQSISSWLAWYQQKPGKAPKLLIYDASSLESGVPSRFS |
| | GSGSGTEFTLTISSLQPDDFATYYCQQYGIPWTFGQGTKVEIKR |
| 128 | DIQMTQSPSTLSASVGDRVTITCRASQSISSWLAWYQQKPGKAPKLLIYDASSLESGVPSRFS |
| | GSGSGTEFTLTISSLQPDDFATYYCQQYTVYSPVTFGQGTKVEIKR |
| 129 | QVQLVQSGAEVKKPGSSVKVSCKASGGTFSSYAISWVRQAPGQGLEWMGGIIPIFGTANYAQ |
| | KFQGRVTITADKSTSTAYMELSSLRSEDTAVYYCARGYFSEEAMDYWGQGTTVTVSS |
| 130 | QVQLVQSGAEVKKPGSSVKVSCKASGGTFSSYAISWVRQAPGQGLEWMGGIIPIFGTANYAQ |
| | KFQGRVTITADKSTSTAYMELSSLRSEDTAVYYCARAWYFGYDFDYWGQGTTVTVSS |
| 131 | QVQLVQSGAEVKKPGSSVKVSCKASGGTFSSYAISWVRQAPGQGLEWMGGIIPIFGTANYAQ |
| | KFQGRVTITADKSTSTAYMELSSLRSEDTAVYYCARGWYFFDAGFDYWGQGTTVTVSS |
| 132 | QVQLVQSGAEVKKPGSSVKVSCKASGGTFSSYAISWVRQAPGQGLEWMGGIIPIFGTANYAQ |
| | KFQGRVTITADKSTSTAYMELSSLRSEDTAVYYCAREWYWPDNPFDYWGQGTTVTVSS |
| 133 | QVQLVQSGAEVKKPGSSVKVSCKASGGTFSSYAISWVRQAPGQGLEWMGGIIPIFGTANYAQ |
| | KFQGRVTITADKSTSTAYMELSSLRSEDTAVYYCARAPYSPTYYPFDYWGQGTTVTVSS |
| 134 | EVQLLESGGGLVQPGGSLRLSCAASGFTFSSYAMSWVRQAPGKGLEWVSAISGSGGSTYYA |
| | DSVKGRFTISRDNSKNTLYLQMNSLRAEDTAVYYCARSYNWYEWYWFDYWGQGTLVTVSS |
| 135 | EVQLLESGGGLVQPGGSLRLSCAASGFTFSSYAMSWVRQAPGKGLEWVSAISGSGGSTYYA |
| | DSVKGRFTISRDNSKNTLYLQMNSLRAEDTAVYYCARDWAGIYYLDYWGQGTLVTVSS |
| 136 | EVQLLESGGGLVQPGGSLRLSCAASGFTFSSYAMSWVRQAPGKGLEWVSAISGSGGSTYYA |
| | DSVKGRFTISRDNSKNTLYLQMNSLRAEDTAVYYCARDWGGIYYFDYWGQGTLVTVSS |
| 137 | EVQLLESGGGLVQPGGSLRLSCAASGFTFSSYAMSWVRQAPGKGLEWVSAISGSGGSTYYA |
| | DSVKGRFTISRDNSKNTLYLQMNSLRAEDTAVYYCARPSLADWGDMDYWGQGTLVTVSS |
| 138 | EVQLLESGGGLVQPGGSLRLSCAASGFTFSSYAMSWVRQAPGKGLEWVSAISGSGGSTYYA |
| | DSVKGRFTISRDNSKNTLYLQMNSLRAEDTAVYYCAKDWVYFGVGSFDYWGQGTLVTVSS |
| 139 | EVQLLESGGGLVQPGGSLRLSCAASGFTFSSYAMSWVRQAPGKGLEWVSAISGSGGSTYYA |
| | DSVKGRFTISRDNSKNTLYLQMNSLRAEDTAVYYCARDYWLDSDYFDYWGQGTLVTVSS |
| 140 | EVQLLESGGGLVQPGGSLRLSCAASGFTFSSYAMSWVRQAPGKGLEWVSAISGSGGSTYYA |
| | DSVKGRFTISRDNSKNTLYLQMNSLRAEDTAVYYCARDYGTISAFDYWGQGTLVTVSS |
| 141 | EVQLLESGGGLVQPGGSLRLSCAASGFTFSSYAMSWVRQAPGKGLEWVSAISGSGGSTYYA |
| | DSVKGRFTISRDNSKNTLYLQMNSLRAEDTAVYYCAKEYFYAMDYWGQGTLVTVSS |
| 142 | agagtctctcagctggtaca |
| 143 | gagtagcgcgagcacagcta |
| 144 | gcacttggtattctgggtcc |

1-87. (canceled)

88. An engineered cell targeting allogeneic immune cells, wherein the engineered cell expresses a molecule that recognizes a NKG2D ligand, and the expression of the molecule that recognizes the NKG2D ligand is regulatable.

89. The engineered cell of claim 88, wherein the molecule that recognizes the NKG2D ligand comprises: a bispecific molecule, a peptide-drug conjugates, and a first chimeric receptor;
optionally, the molecule that recognizes the NKG2D ligand comprises a full-length NKG2D polypeptide or a fragment thereof, or an antibody that recognizes a NKG2D ligand or a fragment thereof; preferably, the NKG2D polypeptide comprises the sequence represented by SEQ ID NO: 42;
optionally, the first chimeric receptor comprises an extracellular domain, a transmembrane domain and an intracellular domain; the extracellular domain comprises an extracellular domain of a NKG2D polypeptide, or an antibody that recognizes a NKG2D ligand or a fragment thereof; preferably, the extracellular domain is connected to the transmembrane domain via a hinge;
optionally, the intracellular domain of the first chimeric receptor comprises an immunoreceptor tyrosine-based activation motif or an ITAM signaling transduction motif;
optionally, the intracellular domain of the first chimeric receptor is selected from: the intracellular signaling domain of TCRε, FcRγ, FcRβ, CD3γ, CD3δ, CD3ε, CD3ζ, CD5, CD22, CD79a, CD79b, CD278, CD66d, CD28, CD137, or any combination thereof;
optionally, the transmembrane domain of the first chimeric receptor is selected from: the transmembrane domain of TCRα chain, TCRβ chain, TCRγ chain, TCRδ chain, CD3ζ subunit, CD3ε subunit, CD3γ subunit, CD3ζ subunit, BAFFR, CEACAM1, CD100 (SEMA4D), CD103, CD11a, CD11b, CD11c, CD11d, CD137 (4-1BB), CD16, CD160, CD18 (CD11a, LFA-1), CD160(BY55), CD162(SELPLG), CD19, CD2, CD22, CD226(DNAM1), CD229(Ly9), CD244(SLAMF4, 2B4), CD27, CD278(ICOS), CD28, CD29, CD33, CD37, CD4, CD40, CD45, CD49a, CD49D, CD49f, CD5, CD64, CD8, CD80, CD84, CD86, CD9, CD96 (Tactile), CD134, CD154, CRTAM, GITR, HLA-E, HLA-F, HLA-G, HVEM (LIGHTR), IA4, IL2Rβ, IL2Rγ, IL7Rα, ITGA1, ITGA4, ITGA6, ITGAD, ITGAE, ITGAL, ITGAM, ITGAX, ITGB1, ITGB2, ITGB7, KIRDS2, LTBR, NKp80(KLRF1), NKp44, NKp30, NKp46, NKG2D, NKG2C, OCIL, OX40, PAG/Cbp, PSGL1, SLAM (SLAMF1, CD150, IPO-3), SLAMF6 (NTB-A, Ly108), SLAMF7, BLAME(SLAMF8), TNFR2, VLA1, VLA-6, cadherin and/or collagen;
optionally, the extracellular domain of the first chimeric receptor comprises a extracellular domain of a NKG2D polypeptide, or an antibody that recognizes a NKG2D ligand or a fragment thereof, the transmembrane domain comprises a NKG2D transmembrane domain, a CD8 transmembrane domain or a CD28 transmembrane domain, and the intracellular domain comprises a NKG2D polypeptide intracellular domain, a CD28 intracellular domain, a CD137 intracellular domain, a CD3ζ intracellular domain or any combination thereof; optionally, the engineered cell further expresses a DAP10 polypeptide;
optionally, the first chimeric receptor comprises a full-length NKG2D polypeptide; preferably, the intracellular domain of the first chimeric receptor further comprises a CD28 intracellular domain, a CD137 intracellular domain, a CD32 intracellular domain, or any combination thereof; more preferably, the first chimeric receptor comprises the sequence represented by SEQ ID NO: 51, 52, 53 and/or 54.

90. The engineered cell of claim 88, wherein the engineered cell is an immune cell, a neuron, an epithelial cell, an endothelial cell or a stem cell;
optionally, the engineered cell is an autologous or allogeneic T cell, NK cell, NKT cell, macrophage, CIK cell, or stem cell-derived immune cell; preferably, the engineered cell is an autologous or allogeneic T cell.

91. The engineered cell of claim 88, wherein the engineered cell further expresses a second chimeric polypeptide that does not recognizes the NKG2D ligand, and the second chimeric polypeptide induces the expression of the molecule that recognizes the NKG2D ligand after recognizing a first target molecule;
preferably, after the binding domain of the second chimeric polypeptide binds to the first target molecule, the second chimeric polypeptide is induced to cleave, releasing a transcription factor, and the transcription factor regulates the expression of the molecule that recognizes the NKG2D ligand;
optionally, the second chimeric polypeptide comprises:
a) a binding domain capable of specifically binding to a first target molecule;
b) a receptor regulatory domain comprising a cleavage site,
the receptor regulatory domain comprises an extracellular domain and a transmembrane domain, wherein the extracellular domain comprises an EphrinB2 extracellular domain or a fragment thereof, and the transmembrane domain comprises one or more target molecule binding-induced cleavage sites; and
c) an intracellular domain,
wherein, the binding of the binding domain to the first target molecule can induce the cleavage of the receptor regulatory domain, thereby releasing the intracellular domain;
optionally, the second chimeric polypeptide comprises a synNotch polypeptide; or the second chimeric polypeptide comprises an amino acid sequence of any one of SEQ ID NO: 44, 45, 46, 47, 48, 49, 60, 61, 62, 63, 64, 65, 66 or 67;
optionally, the first target molecule recognized by the second chimeric polypeptide comprises a tumor antigen, a tissue-specific marker, and/or a pathogen antigen;
optionally, the engineered cell further expresses a third chimeric receptor that recognizes a tumor antigen and/or a pathogen antigen;
optionally, the third chimeric receptor comprises a chimeric antigen receptor (CAR) and/or a recombinant T cell receptor (a recombinant TCR);
optionally, the tissue-specific antigen comprises: a brain tissue-specific marker MOG, a liver tissue-specific marker ASGR1, a prostate tissue marker PSA, or any combination thereof;
optionally, the tumor antigen is selected from the group consisting of: ALPPL2, ALPI, Axl, B7H3, BCMA, CD117, CD123, CD171, CD179a, CD19, CD213A2, CD20, CD22, CD24, CD246, CD272, CD30, CD33, CD38, CD44v6, CD46, CD7, CD71, CD94, CD97, CEA, Claudin18.2, CLDN6, CLECL1, CLL1, cMet, CS-1, EGFR, EGFRVIII, ELF2M, EpCAM, EphA2, FAP, FCRH5, FLT3, GD2, GD3, GM3, GPC3, GPRC5D, HER2(ERBB2), IGLL1, IL 11Ra, IL13Ra2, Mesothelin, MUC1, NCAM, NKG2D-Ligand, PAP, PDGFR-b, PRSS21, PSCA, PSMA, ROR1, SIRPa, SSEA-4, TAG72, TEM1/CD248, TEM7R, TSHR, VEGFR2 and WT1;
optionally, the engineered cells target an allogeneic NK, NKT and/or T cell, or the engineered cells kill an allogeneic NK, NKT and/or T cell or resist immune rejection of an allogeneic NK, NKT and/or T cell;
optionally, the first chimeric receptor and the second chimeric polypeptide are located in a same expression vector; preferably, the vector comprises the nucleotide sequence represented by SEQ ID NO: 55, 56, or 57;
optionally, the endogenous B2M, TCR, MHC-II, FAS, NKG2A and/or NKG2D ligand molecules of the engineered cell are in a state of low expression or no expression, or a combination thereof;
preferably, the endogenous B2M/TCR/FAS of the engineered cell is in a state of low expression or no expression;
preferably, the sgRNA sequences targeting TRAC, B2M, and FAS are represented by SEQ ID NOs: 142, 143, and 144, respectively;
optionally, the engineered cell has improved survival and proliferation capabilities during in vivo and in vitro culture compare to a reference cell.

92. The engineered cell of claim 88, wherein the engineered cell is administered in combination with a second immune cell expressing a fourth chimeric receptor that recognizes a tumor antigen and/or a pathogen antigen;
optionally, the engineered cell enhances the survival or proliferation of the second immune cell administered previously, simultaneously, or subsequently; or enhances the killing of a tumor cell by the second immune cell.

93. A cell composition, comprising the engineered cell of claim 88, and a second immune cell expressing a fourth chimeric receptor that recognizes a tumor antigen and/or a pathogen antigen, preferably, the antigen recognized by the fourth chimeric receptor is different from the antigen recognized by the second chimeric polypeptide;
optionally, the tumor antigen recognized by the fourth chimeric receptor is selected from the group consisting of: ALPPL2, ALPI, Axl, B7H3, BCMA, CD 117, CD123, CD171, CD179a, CD19, CD213A2, CD20, CD22, CD24, CD246, CD272, CD30, CD33, CD38, CD44v6, CD46, CD7, CD71, CD94, CD97, CEA, Claudin18.2, CLDN6, CLECL1, CLL1, cMet, CS-1, EGFR, EGFRVIII, ELF2M, EpCAM, EphA2, FAP, FCRH5, FLT3, GD2, GD3, GM3, GPC3, GPRC5D, HER2 (ERBB2), IGLL1, IL11Ra, IL13Ra2, Mesothelin, MUC1, NCAM, NKG2D-Ligand, PAP, PDGFR-b, PRSS21, PSCA, PSMA, ROR1, SIRPa, SSEA-4, TAG72, TEM1/CD248, TEM7R, TSHR, VEGFR2 and WT1;
optionally, the fourth chimeric receptor comprises a chimeric antigen receptor (CAR) and/or a recombinant T cell receptor (a recombinant TCR);
optionally, the endogenous B2M, TCR, MHC-II, FAS, NKG2A and/or NKG2D ligand molecules of the engineered cell and the second immune cell are in a state of low expression or no expression, or a combination thereof;
preferably, the endogenous B2M/TCR/FAS of the engineered cell is in a state of low expression or no expression;
preferably, the sgRNA sequences targeting TRAC, B2M, and FAS are represented by SEQ ID NOs: 142, 143, and 144, respectively;
optionally, the engineered cell and/or the second immune cell are an autologous or allogeneic T cell, NK cell, and/or NKT cell.

94. A polynucleotide, wherein the polynucleotide encodes the bispecific molecule, the first chimeric receptor, the second chimeric polypeptide, the third chimeric receptor, the fourth chimeric receptor, or any combination thereof comprised by the engineered cell of claim 88.

95. A vector, wherein the vector comprises the polynucleotide of claim 94.

96. Use of the engineered cell of claim 88 for eliminating an autologous or allogeneic NK cell, NKT cell and/or T cell.

97. Use of the engineered cell of claim 88 for preparing an anti-tumor medicament;
optionally, the tumor comprises: a hematologic tumor and/or a solid tumor;
preferably, the hematologic tumor is selected from: leukemia, lymphoma, myeloma, or any combination thereof; the solid tumor is selected from: liver cancer, gastric cancer, esophageal cancer, gastroesophageal junction tumor, pancreatic cancer, bile duct cancer, gallbladder cancer, small intestine cancer, colon cancer, rectal cancer, lung cancer, breast cancer, prostate cancer, ovarian cancer, glioma, melanoma, or any combination thereof.

98. A method for preventing or regulating transplant immune rejection, wherein the engineered cell of claim 88 is administered;
preferably, the method is used to kill an autologous or allogeneic NK cell, NKT cell and/or T cell.

99. A method for increasing the survival time and/or expansion capacity of an engineered cell targeting a tumor and/or pathogen in a subject in the presence of a host immune cell, wherein the engineered cell of claim 88 is administered to the subject; preferably, the subject is a human; preferably, wherein the engineered cell is an autologous or allogeneic T cell, NK cell, and/or NKT cell; preferably, the host immune cell is a NK cell, NKT cell and/or T cell.

100. A pharmaceutical composition, comprising the engineered cell of claim 88 and a pharmaceutically acceptable excipient.

101. An antibody recognizing myelin oligodendrocyte glycoprotein (MOG), wherein the antibody is selected from the group consisting of:
(1) an antibody, comprising a light chain variable domain, wherein the light chain variable domain comprises LCDR1 represented by SEQ ID NO: 84, and/or LCDR2 represented by SEQ ID NO: 85, and/or LCDR3 represented by any one of SEQ ID NO: 86, 87, 88, 89, 90, 91, 92, 93, 94, 95, 96, 97 or 98;
(2) an antibody, comprising a heavy chain variable domain, wherein the heavy chain variable domain comprises HCDR1 represented by SEQ ID NO: 99 or 100, and/or HCDR2 represented by SEQ ID NO: 101 or 102, and/or HCDR3 represented by any one of SEQ ID NO: 103, 104, 105, 106, 107, 108, 109, 110, 111, 112, 113, 114 or 115;
(3) an antibody, comprising the light chain variable domain of the antibody described in (1) and the heavy chain variable domain of the antibody described in (2); and
(4) an antibody, which is a variant of the antibody of any one of (1)-(3), and has the same or similar activity as the antibody of any one of (1)-(3);
optionally, the LCDR1, LCDR2, LCDR3, HCDR1, HCDR2, and HCDR3 comprised by the antibody are respectively selected from:
(1) the amino acid sequences represented by SEQ ID NO: 84, 85, 86, 99, 101, and 103; or
(2) the amino acid sequences represented by SEQ ID NO: 84, 85, 87, 99, 101, and 104; or
(3) the amino acid sequences represented by SEQ ID NO: 84, 85, 88, 99, 101, and 105; or
(4) the amino acid sequences represented by SEQ ID NO: 84, 85, 89, 99, 101, and 106; or
(5) the amino acid sequences represented by SEQ ID NO: 84, 85, 90, 99, 101, and 107; or
(6) the amino acid sequences represented by SEQ ID NO: 84, 85, 91, 100, 102, and 108; or
(7) the amino acid sequences represented by SEQ ID NO: 84, 85, 92, 100, 102, and 109; or
(8) the amino acid sequences represented by SEQ ID NO: 84, 85, 93, 100, 102, and 110; or
(9) the amino acid sequences represented by SEQ ID NO: 84, 85, 94, 100, 102, and 111; or
(10) the amino acid sequences represented by SEQ ID NO: 84, 85, 95, 100, 102, and 112; or
(11) the amino acid sequences represented by SEQ ID NO: 84, 85, 96, 100, 102, and 113; or
(12) the amino acid sequences represented by SEQ ID NO: 84, 85, 97, 100, 102, and 114; or
(13) the amino acid sequences represented by SEQ ID NOs: 84, 85, 98, 100, 102, and 115.

102. The antibody of claim 101, wherein the antibody is selected from the group consisting of:
(1) an antibody, comprising a light chain variable domain, wherein the light chain variable domain comprises an amino acid sequence represented by SEQ ID NO: 116, 117, 118, 119, 120, 121, 122, 123, 124, 125, 126, 127 or 128, or a nucleic acid sequence encoding the amino acid sequence, or a variant of any one of the above sequences, or an amino acid sequence having at least 80% identity with any one of the above sequences, or a nucleic acid sequence encoding the amino acid sequence;
(2) an antibody, comprising a heavy chain variable domain, wherein the heavy chain variable domain comprises an amino acid sequence represented by SEQ ID NO: 129, 130, 131, 132, 133, 134, 135, 136, 137, 138, 139, 140 or 141, or a nucleic acid sequence encoding the amino acid sequence, or a variant of any one of the above sequences, or an amino acid sequence having at least 80% identity with any one of the above sequences, or a nucleic acid sequence encoding the amino acid sequence; and
(3) an antibody comprising the light chain variable domain of the antibody described in (1) and the heavy chain variable domain of the antibody described in (2);
optionally, the light chain variable domain and the heavy chain variable domain of the antibody are respectively selected from: amino acid sequences of SEQ ID NOs: 116 and 129; SEQ ID NOs: 117 and 130; SEQ ID NOs: 118 and 131; SEQ ID NOs: 119 and 132; SEQ ID NOs: 120 and 133; SEQ ID NOs: 121 and 134; SEQ ID NOs: 122 and 135; SEQ ID NOs: 123 and 136; SEQ ID NOs: 124 and 137; SEQ ID NOs: 125 and 138; SEQ ID NOs: 126 and 139; SEQ ID NOs: 127 and 140; or SEQ ID NOs: 128 and 141; or amino acid sequences having 90%, 91%, 92%, 93%, 94%, 95%, 96%, 97%, 98%, or 99% identity with the above amino acid sequences, or nucleic acid sequences encoding the amino acid sequences;
optionally, the scFv of the antibody has an amino acid sequence represented by SEQ ID NO: 43, 68, 77, 81, 82 or 83, or an amino acid sequence having 90%, 91%, 92%, 93%, 94%, 95%, 96%, 97%, 98%, or 99% identity with the above sequence, or a nucleic acid sequence encoding the amino acid sequence;
optionally, the antibody is a full-length antibody, a scFv, a single domain antibody, a Fab fragment, a Fab′ fragment, a Fv fragment, a F(ab′)2 fragment, a Fd fragment, a dAb fragment, a multifunctional antibody, a scFv-Fc antibody or an IgG4 antibody;
optionally, the antibody binds to human or mouse MOG; and/or the antibody binds to a cell expressing human or mouse MOG;
optionally, the antibody is a fully human antibody.

103. An immunoconjugate, wherein the immunoconjugate comprises: the antibody of claim 101, and a functional molecule linked thereto.

104. A chimeric polypeptide, wherein
a) the binding domain of the chimeric polypeptide comprises the antibody of claim 101;
b) a receptor regulatory domain comprising one or more cleavage sites, the receptor regulatory domain comprises an extracellular domain and a transmembrane domain; and
c) an intracellular domain,
wherein, the binding of the binding domain to MOG can induce the cleavage of the receptor regulatory domain, thereby releasing the intracellular domain.

105. A biological material, which is any one of the following:
1) a nucleic acid, encoding the antibody of claim 101, or a chimeric polypeptide, wherein
a) the binding domain of the chimeric polypeptide comprises the antibody of claim 101;
b) a receptor regulatory domain comprising one or more cleavage sites, the receptor regulatory domain comprises an extracellular domain and a transmembrane domain; and
c) an intracellular domain,
wherein, the binding of the binding domain to MOG can induce the cleavage of the receptor regulatory domain, thereby releasing the intracellular domain;
2) an expression vector comprising the nucleic acid of 1); or
3) a virus comprising the nucleic acid of 1) or the expression vector of 2).

106. An engineered cell comprising the chimeric polypeptide of claim 104.

107. A pharmaceutical composition, comprising:
the antibody of claim 101.


SEQUENCE LISTING (144 sequences)

SEQ ID NO: 1 - protein, 65 aa, synthetic construct
GQDASSAGSTRNKDPTRRPELEAGTNGRSSTTSPFVKPNPGSSTDGNSAGHSGNNILGSEVALFA

SEQ ID NO: 2 - DNA, 195 bp, synthetic construct
  source 1..195: mol_type = other DNA
ggacaagatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgca

SEQ ID NO: 3 - protein, 54 aa, synthetic construct
GQDATRRPELEAGTNGRSSTTSPFVKPNPGSSTDGNSAGHSGNNILGSEVALFA

SEQ ID NO: 4 - protein, 52 aa, synthetic construct
GQDASSAGSTRNKDPTRRPSPFVKPNPGSSTDGNSAGHSGNNILGSEVALFA

SEQ ID NO: 5 - protein, 41 aa, synthetic construct
GQDASSAGSTRNKDPTRRPELEAGTNGRSSTTSPSEVALFA

SEQ ID NO: 6 - protein, 26 aa, synthetic construct
GQDASSAGSTRNKDPTRRPSEVALFA

SEQ ID NO: 7 - protein, 105 aa, synthetic construct
ASSPCLGGNPCYNQGTCEPTSESPFYRCLCPAKFNGLLCHGQDASSAGSTRNKDPTRRPELEAGTNGRSSTTSPFVKPNPGSSTDGNSAGHSGNNILGSEVALFA

SEQ ID NO: 8 - protein, 144 aa, synthetic construct
DARTCGSLRCLNGGTCISGPRSPTCLCLGPFTGPECQFPASSPCLGGNPCYNQGTCEPTSESPFYRCLCPAKFNGLLCHGQDASSAGSTRNKDPTRRPELEAGTNGRSSTTSPFVKPNPGSSTDGNSAGHSGNNILGSEVALFA

SEQ ID NO: 9 - protein, 185 aa, synthetic construct
VINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHGQDASSAGSTRNKDPTRRPELEAGTNGRSSTTSPFVKPNPGSSTDGNSAGHSGNNILGSEVALFA

SEQ ID NO: 10 - protein, 225 aa, synthetic construct
DVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHGQDASSAGSTRNKDPTRRPELEAGTNGRSSTTSPFVKPNPGSSTDGNSAGHSGNNILGSEVALFA

SEQ ID NO: 11 - protein, 27 aa, synthetic construct
LMYVAAAAFVLLFFVGCGVLLSRKRRR

SEQ ID NO: 12 - protein, 29 aa, synthetic construct
LLYLLAVAVVIILFFILLGVIMAKRKRKH

SEQ ID NO: 13 - protein, 29 aa, synthetic construct
LLPLLVAGAVFLLIIFILGVMVARRKREH

SEQ ID NO: 14 - protein, 31 aa, synthetic construct
PILCSPVVGVLLLALGALLVLQLIRRRRREH

SEQ ID NO: 15 - protein, 27 aa, synthetic construct
FMYVAAAAFVLLFFVGCGVLLSRKRRR

SEQ ID NO: 16 - protein, 29 aa, synthetic construct
LLYLLAVAVVIILFIILLGVIMAKRKRKH

SEQ ID NO: 17 - protein, 28 aa, synthetic construct
LPLLVAGAVLLLVILVLGVMVARRKREH

SEQ ID NO: 18 - protein, 31 aa, synthetic construct
PVLCSPVAGVILLALGALLVLQLIRRRRREH

SEQ ID NO: 19 - protein, 28 aa, synthetic construct
LMYVAAAAFVLLFFVGCGVLLSKRKRKH

SEQ ID NO: 20 - protein, 28 aa, synthetic construct
LMYVAAAAFVLLFFVGCGVLLSRRKREH

SEQ ID NO: 21 - protein, 29 aa, synthetic construct
LMYVAAAAFVLLFFVGCGVLLSRRRRREH

SEQ ID NO: 22 - protein, 28 aa, synthetic construct
SALIGLLVIAVAIATVIVISLVMLRKRQ

SEQ ID NO: 23 - protein, 5 aa, synthetic construct
RKRRR

SEQ ID NO: 24 - protein, 6 aa, synthetic construct
KRKRKH

SEQ ID NO: 25 - protein, 6 aa, synthetic construct
RRKREH

SEQ ID NO: 26 - protein, 7 aa, synthetic construct
RRRRREH

SEQ ID NO: 27 - protein, 4 aa, synthetic construct
RKRQ

SEQ ID NO: 28 - protein, 211 aa, synthetic construct
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 29 - DNA, 144 bp, synthetic construct
  source 1..144: mol_type = other DNA
aagcttgatatcgaattcggagcactgtcctccgaacgtcggagcactgtcctccgaacgtcggagcactgtcctccgaacgtcggagcactgtcctccgaacggagcatgtcctccgaacgtcggagcactgtcctccgaacg

SEQ ID NO: 30 - DNA, 288 bp, synthetic construct
  source 1..288: mol_type = other DNA
aagcttgatatcgaattcggagcactgtcctccgaacgtcggagcactgtcctccgaacgtcggagcactgtcctccgaacgtcggagcactgtcctccgaacggagcatgtcctccgaacgtcggagcactgtcctccgaacgactagttaggcgtgtacggtgggaggcctatataagcagagctcgtttagtgaaccgtcagatcgcctggagacgccatccacgctgttttgacctccatagaagacaccgggaccgatccagcctctcgacattcgttggatc

SEQ ID NO: 31 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 32 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 33 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 34 - DNA, 123 bp, synthetic construct
  source 1..123: mol_type = other DNA
aggagtaagaggagcaggctcctgcacagtgactacatgaacatgactccccgccgccccgggccaacccgcaagcattaccagccctatgccccaccacgcgacttcgcagcctatcgctcc

SEQ ID NO: 35 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 36 - DNA, 339 bp, synthetic construct
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 37 - DNA, 63 bp, synthetic construct
  source 1..63: mol_type = other DNA
atggccttaccagtgaccgccttgctcctgccgctggccttgctgctccacgccgccaggccg

SEQ ID NO: 38 - protein, 226 aa, synthetic construct
TTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 39 - protein, 224 aa, synthetic construct
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - protein, 268 aa, synthetic construct
TTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 41 - protein, 247 aa, synthetic construct
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 42 - protein, 216 aa, synthetic construct
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 43 - protein, 241 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEYFYAMDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYTVYSPVTFGQGTKVEIKR

SEQ ID NO: 44 - protein, 556 aa, synthetic construct
EQKLISEEDLEVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRGQDASSAGSTRNKDPTRRPELEAGTNGRSSTTSPFVKPNPGSSTDGNSAGHSGNNILGSEVALFALMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 45 - protein, 532 aa, synthetic construct
EQKLISEEDLEVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRGQDASSAGSTRNKDPTRRPELEAGTNGRSSTTSPSEVALFALMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 46 - protein, 545 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWGGIYYFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQKDTWSYTFGQGTKVEIKRGQDASSAGSTRNKDPTRRPELEAGTNGRSSTTSPFVKPNPGSSTDGNSAGHSGNNILGSEVALFALMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 47 - protein, 521 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWGGIYYFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQKDTWSYTFGQGTKVEIKRGQDASSAGSTRNKDPTRRPELEAGTNGRSSTTSPSEVALFALMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 48 - protein, 545 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDDSFYGGFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYSSYKYTFGQGTKVEIKRGQDASSAGSTRNKDPTRRPELEAGTNGRSSTTSPFVKPNPGSSTDGNSAGHSGNNILGSEVALFALMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 49 - protein, 521 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDDSFYGGFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYSSYKYTFGQGTKVEIKRGQDASSAGSTRNKDPTRRPELEAGTNGRSSTTSPSEVALFALMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 50 - protein, 467 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYRAVTFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQFETQIRWTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 51 - protein, 371 aa, synthetic construct
MRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRKRGRKKLLYIFKQPFMRPVQTTQEEDGCSCRFPEEEEGGCELGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 52 - protein, 329 aa, synthetic construct
MRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 53 - protein, 444 aa, synthetic construct
MRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGSGATNFSLLKQAGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 54 - protein, 370 aa, synthetic construct
IWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 55 - DNA, 3286 bp, synthetic construct
  source 1..3286: mol_type = other DNA
aagcttgatatcgaattcggagcactgtcctccgaacgtcggagcactgtcctccgaacgtcggagcactgtcctccgaacgtcggagcactgtcctccgaacggagcatgtcctccgaacgtcggagcactgtcctccgaacgactagttaggcgtgtacggtgggaggcctatataagcagagctcgtttagtgaaccgtcagatcgcctggagacgccatccacgctgttttgacctccatagaagacaccgggaccgatccagcctctcgacattcgttgatcatatgccaccatgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtagctatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtgtaagctagcatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccaggcctaagcttacgcgtcctagcgctaccggtcgccaccatggccttaccagtgaccgccttgctcctgccgctggccttgctgctccacgccgccaggccggaggtgcaattgctggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaagatgattctttctacggtggtttcgactactggggccaaggaaccctggtcaccgtctcgagtggtggaggcggttcaggcggaggtggttctggcggtggcggatcggacatccagatgacccagtctccttccaccctgtctgcatctgtaggagaccgtgtcaccatcacttgccgtgccagtcagagtattagtagctggttggcctggtatcagcagaaaccagggaaagcccctaagctcctgatctatgatgcctccagtttggaaagtggggtcccatcacgtttcagcggcagtggatccgggacagaattcactctcaccatcagcagcttgcagcctgatgattttgcaacttattactgccaacagtactcttcttacaaatacacgtttggccagggcaccaaagtcgagatcaagcgtggacaagatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgcactcatgtacgtggcagcggccgccttcgtgctcctgttctttgtgggctgtggggtgctgctgtcccgcaagcgccggcggatgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatca

SEQ ID NO: 56 - DNA, 3398 bp, synthetic construct
  source 1..3398: mol_type = other DNA
aagcttgatatcgaattcggagcactgtcctccgaacgtcggagcactgtcctccgaacgtcggagcactgtcctccgaacgtcggagcactgtcctccgaacggagcatgtcctccgaacgtcggagcactgtcctccgaacgactagttaggcgtgtacggtgggaggcctatataagcagagctcgtttagtgaaccgtcagatcgcctggagacgccatccacgctgttttgacctccatagaagacaccgggaccgatccagcctctcgacattcgttggatcatattgccaccatgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcaaacggggcagaaagaaactcctgtatatattcaaacaaccatttatgagaccagtacaaactactcaagaggaagatggctgtagctgccgatttccagaagaagaagaaggaggatgtgaactggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtagctatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacttacatctgcatgcaaaggactgtgtaagctagcatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccaggcctaagcttacgcgtgccaccatggccttaccagtgaccgccttgctcctgccgctggccttgctgctccacgccgccaggccggaggtgcaattgctggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaagatgattctttctacggtggtttcgactactggggccaaggaaccctggtcaccgtctcgagtggtggaggcggttcaggcggaggtggttctggcggtggcggatcggacatccagatgacccagtctccttccaccctgtctgcatctgtaggagaccgtgtcaccatcacttgccgtgccagtcagagtattagtagctggttggcctggtatcagcagaaaccagggaaagcccctaagctcctgatctatgatgcctccagtttggaaagtggggtcccatcacgtttcagcggcagtggatccgggacagaattcactctcaccatcagcagcttgcagcctgatgattttgcaacttattactgccaacagtactcttcttacaaatacacgtttggccagggcaccaaagtcgagatcaagcgtggacaagatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgcactcatgtacgtggcagcggccgccttcgtgctcctgttctttgtgggctgtggggtgctgctgtcccgcaagcgccggcggatgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatca

SEQ ID NO: 57 - DNA, 3632 bp, synthetic construct
  source 1..3632: mol_type = other DNA
aagcttgatatcgaattcggagcactgtcctccgaacgtcggagcactgtcctccgaacgtcggagcactgtcctccgaacgtcggagcactgtcctccgaacggagcatgtcctccgaacgtcggagcactgtcctccgaacgactagttaggcgtgtacggtgggaggcctatataagcagagctcgtttagtgaaccgtcagatcgcctggagacgccatccacgctgttttgacctccatagaagacaccgggaccgatccagcctctcgacattcgttggatcatatgccaccatgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtagctatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtgggaagcggagctactaacttcagcctgctgaagcaggctggagacgtggaggagaaccctggacctatgatccatctgggtcacatcctcttcctgcttttgctcccagtggctgcagctcagacgactccaggagagagatcatcactccctgccttttaccctggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggctaagctagcatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccaggcctaagcttacgcgtcctagcgctaccggtcgccaccatggccttaccagtgaccgccttgctcctgccgctggccttgctgctccacgccgccaggccggaggtgcaattgctggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaagatgattctttctacggtggtttcgactactggggccaaggaaccctggtcaccgtctcgagtggtggaggcggttcaggcggaggtggttctggcggtggcggatcggacatccagatgacccagtctccttccaccctgtctgcatctgtaggagaccgtgtcaccatcacttgccgtgccagtcagagtattagtagctggttggcctggtatcagcagaaaccagggaaagcccctaagctcctgatctatgatgcctccagtttggaaagtggggtcccatcacgtttcagcggcagtggatccgggacagaattcactctcaccatcagcagcttgcagcctgatgattttgcaacttattactgccaacagtactcttcttacaaatacacgtttggccagggcaccaaagtcgagatcaagcgtggacaagatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgcactcatgtacgtggcagcggccgccttcgtgctcctgttctttgtgggctgtggggtgctgctgtcccgcaagcgccggcggatgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatca

SEQ ID NO: 58 - DNA, 3755 bp, synthetic construct
  source 1..3755: mol_type = other DNA
aagcttgatatcgaattcggagcactgtcctccgaacgtcggagcactgtcctccgaacgtcggagcactgtcctccgaacgtcggagcactgtcctccgaacggagcatgtcctccgaacgtcggagcactgtcctccgaacgactagttaggcgtgtacggtgggaggcctatataagcagagctcgtttagtgaaccgtcagatcgcctggagacgccatccacgctgttttgacctccatagaagacaccgggaccgatccagcctctcgacattcgttggatcatggccttaccagtgaccgccttgctcctgccgctggccttgctgctccacgccgccaggccggaggtgcaattgctggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaataccgtgcagttactttcgactactggggccaaggaaccctggtcaccgtctcgagtggtggaggcggttcaggcggaggtggttctggcggtggcggatcggacatccagatgacccagtctccttccaccctgtctgcatctgtaggagaccgtgtcaccatcacttgccgtgccagtcagagtattagtagctggttggcctggtatcagcagaaaccagggaaagcccctaagctcctgatctatgatgcctccagtttggaaagtggggtcccatcacgtttcagcggcagtggatccgggacagaattcactctcaccatcagcagcttgcagcctgatgattttgcaacttattactgccaacagttcgaaacccagatcagatggacgtttggccagggcaccaaagtcgagatcaagcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggccaacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagtaagctagccgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccaggcctaagcttacgcgtcctagcgctaccggtcgccaccatggcgctccctgtcaccgcactgcttcttccgctggcactgctgctgcacgctgcacggcctgaggtgcaattgctggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgcgtgattggggtggtatctactacttcgactactggggccaaggaaccctggtcaccgtctcgagtggtggaggcggttcaggcggaggtggttctggcggtggcggatcggacatccagatgacccagtctccttccaccctgtctgcatctgtaggagaccgtgtcaccatcacttgccgtgccagtcagagtattagtagctggttggcctggtatcagcagaaaccagggaaagcccctaagctcctgatctatgatgcctccagtttggaaagtggggtcccatcacgtttcagcggcagtggatccgggacagaattcactctcaccatcagcagcttgcagcctgatgattttgcaacttattactgccaacagaaagatacctggtcttacacgtttggccagggcaccaaagtcgagatcaagcgtggacaagatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgcactcatgtacgtggcagcggccgccttcgtgctcctgttctttgtgggctgtggggtgctgctgtcccgcaagcgccggcggatgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatca

SEQ ID NO: 59 - DNA, 3683 bp, synthetic construct
  source 1..3683: mol_type = other DNA
aagcttgatatcgaattcggagcactgtcctccgaacgtcggagcactgtcctccgaacgtcggagcactgtcctccgaacgtcggagcactgtcctccgaacggagcatgtcctccgaacgtcggagcactgtcctccgaacgactagttaggcgtgtacggtgggaggcctatataagcagagctcgtttagtgaaccgtcagatcgcctggagacgccatccacgctgttttgacctccatagaagacaccgggaccgatccagcctctcgacattcgttggatcatggccttaccagtgaccgccttgctcctgccgctggccttgctgctccacgccgccaggccggaggtgcaattgctggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaataccgtgcagttactttcgactactggggccaaggaaccctggtcaccgtctcgagtggtggaggcggttcaggcggaggtggttctggcggtggcggatcggacatccagatgacccagtctccttccaccctgtctgcatctgtaggagaccgtgtcaccatcacttgccgtgccagtcagagtattagtagctggttggcctggtatcagcagaaaccagggaaagcccctaagctcctgatctatgatgcctccagtttggaaagtggggtcccatcacgtttcagcggcagtggatccgggacagaattcactctcaccatcagcagcttgcagcctgatgattttgcaacttattactgccaacagttcgaaacccagatcagatggacgtttggccagggcaccaaagtcgagatcaagcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggccaacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagtaagctagccgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccaggcctaagcttacgcgtcctagcgctaccggtcgccaccatggcgctccctgtcaccgcactgcttcttccgctggcactgctgctgcacgctgcacggcctgaggtgcaattgctggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgcgtgattggggtggtatctactacttcgactactggggccaaggaaccctggtcaccgtctcgagtggtggaggcggttcaggcggaggtggttctggcggtggcggatcggacatccagatgacccagtctccttccaccctgtctgcatctgtaggagaccgtgtcaccatcacttgccgtgccagtcagagtattagtagctggttggcctggtatcagcagaaaccagggaaagcccctaagctcctgatctatgatgcctccagtttggaaagtggggtcccatcacgtttcagcggcagtggatccgggacagaattcactctcaccatcagcagcttgcagcctgatgattttgcaacttattactgccaacagaaagatacctggtcttacacgtttggccagggcaccaaagtcgagatcaagcgtggacaagatgcaagttctgctggatcaaccaggaataaagatccaacaagacgtccagaactagaagctggtacaaatggaagaagttcgacaacaagtccctccgaagtggccttatttgcactcatgtacgtggcagcggccgccttcgtgctcctgttctttgtgggctgtggggtgctgctgtcccgcaagcgccggcggatgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatca

SEQ ID NO: 60 - protein, 92 aa, synthetic construct
GQDASSAGSTRNKDPTRRPELEAGTNGRSSTTSPFVKPNPGSSTDGNSAGHSGNNILGSEVALFALMYVAAAAFVLLFFVGCGVLLSRKRRR

SEQ ID NO: 61 - protein, 81 aa, synthetic construct
GQDATRRPELEAGTNGRSSTTSPFVKPNPGSSTDGNSAGHSGNNILGSEVALFALMYVAAAAFVLLFFVGCGVLLSRKRRR

SEQ ID NO: 62 - protein, 79 aa, synthetic construct
GQDASSAGSTRNKDPTRRPSPFVKPNPGSSTDGNSAGHSGNNILGSEVALFALMYVAAAAFVLLFFVGCGVLLSRKRRR

SEQ ID NO: 63 - protein, 68 aa, synthetic construct
GQDASSAGSTRNKDPTRRPELEAGTNGRSSTTSPSEVALFALMYVAAAAFVLLFFVGCGVLLSRKRRR

SEQ ID NO: 64 - protein, 53 aa, synthetic construct
GQDASSAGSTRNKDPTRRPSEVALFALMYVAAAAFVLLFFVGCGVLLSRKRRR

SEQ ID NO: 65 - protein, 218 aa, synthetic construct
EVIGFGRSCWSRGTPFPHGSSWVEDCNSCRCLDGRRDCSKVWCGWKPCLLAGQPEALSAQCPLGQRCLEKAPGQCLRPPCEAWGECGAEEPPSTPCLPRSGHLDNNCARLTLHFNRDHVPQGTTVGAICSGIRSLPATRAVARDRLLVLLCDRASSGASAVEVAVSFSPARDLPDSSLIQGAAHAIVAAITQRGNSSLLLAVTEVKVETVVTGGSSTG

SEQ ID NO: 66 - protein, 245 aa, synthetic construct
EVIGFGRSCWSRGTPFPHGSSWVEDCNSCRCLDGRRDCSKVWCGWKPCLLAGQPEALSAQCPLGQRCLEKAPGQCLRPPCEAWGECGAEEPPSTPCLPRSGHLDNNCARLTLHFNRDHVPQGTTVGAICSGIRSLPATRAVARDRLLVLLCDRASSGASAVEVAVSFSPARDLPDSSLIQGAAHAIVAAITQRGNSSLLLAVTEVKVETVVTGGSSTGLMYVAAAAFVLLFFVGCGVLLSRKRRR

SEQ ID NO: 67 - protein, 93 aa, synthetic construct
GQDASSAGSTRNKDPTRRPELEAGTNGRSSTTSPFVKPNPGSSTDGNSAGHSGNNILGSEVALFASALIGLLVIAVAIATVIVISLVMLRKRQ

SEQ ID NO: 68 - protein, 242 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWGGIYYFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQKDTWSYTFGQGTKVEIKR

SEQ ID NO: 69 - protein, 243 aa, synthetic construct
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKR

SEQ ID NO: 70 - protein, 241 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYRAVTFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQFETQIRWTFGQGTKVEIKR

SEQ ID NO: 71 - protein, 242 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDDSFYGGFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYSSYKYTFGQGTKVEIKR

SEQ ID NO: 72 - protein, 239 aa, synthetic construct
DIQMTQSPSSLSASVGDRVTITCHASQDINVNIGWLQQKPGKAFKGLIYHGKNLEDGVPSRFSGSGSGTDFTLTISSLQPEDFATYYCNQYENIPLTFGQGTKVEIKRGGGGSGGGGSGGGGSDVQLVESGGGLVQPGGSLRLSCAVSGYSITSDYAWNWIRQAPGKGLEWLGYISYRGRTQYNPSLKSRISITRDNSKNTFFLQLNSLRAEDTAVYYCARMGKNWDYWGQGTLVTVSS

SEQ ID NO: 73 - protein, 247 aa, synthetic construct
QMQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCASSRSGTTVVNHDAFDIWGKGTTVTVSSGGGGSGGGGSGGGGSDIQLTQSPSSLSASVGDRVTITCRASQVISRALAWYQQTPGKPPKLLIYDASNLQSGVPSRFSGSGSGTDFTLTISRLQPEDFATYYCQQFNSYPLTFGGGTKLEIKR

SEQ ID NO: 74 - protein, 244 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDAADRDYWGQGTTVTVSSGGGGSGGGGSGGGGSDIVMTQSPLSLPVTPGEPASISCRSSQSLLHPNGFNHLYWYLQKPGQSPQLLIYVGGNRASGVPDRFSGSGSGTDFTLKISRVEAEDVGVYYCQQRNNKNRTFGQGTKVEIKR

SEQ ID NO: 75 - protein, 247 aa, synthetic construct
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 76 - protein, 243 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGWPSPVTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYKSHPITFGQGTKVEIKR

SEQ ID NO: 77 - protein, 241 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAWYFGYDFDYWGQGTTVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQTAIYSTFGQGTKVEIKR

SEQ ID NO: 78 - protein, 244 aa, synthetic construct
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKR

SEQ ID NO: 79 - protein, 241 aa, synthetic construct
EVQLVQSGAEVKKPGASVKVSCKASGYTFNSYYMHWVRQAPGQGLEWMGIFNPGGGSTSYAQKFQGRVTMTRDTSTSTVYMELSSLRSEDTAVYYCARGTTSDAFDIWGQGTMVTVSSGGGGSGGGGSGGGGSDIQMTQSPSSLSASVGDRVTITCRASQSISSYLNWYQQKPGKAPKLLIDVASSLQSGVPSRFSGSGSGTDFTLTISSLQPEDFATYYCQQSYSTPLTFGGGTKVEIKR

SEQ ID NO: 80 - protein, 247 aa, synthetic construct
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSGGGGSGGGGSGGGGSDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 81 - protein, 244 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARSYNWYEWYWFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYNSFSITFGQGTKVEIKR

SEQ ID NO: 82 - protein, 242 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARPSLADWGDMDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYDQYWTFGQGTKVEIKR

SEQ ID NO: 83 - protein, 245 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDWVYFGVGSFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQTHSYSSTTFGQGTKVEIKR

SEQ ID NO: 84 - protein, 11 aa, synthetic construct
RASQSISSWLA

SEQ ID NO: 85 - protein, 7 aa, synthetic construct
DASSLES

SEQ ID NO: 86 - protein, 8 aa, synthetic construct
QQYDSYYT

SEQ ID NO: 87 - protein, 8 aa, synthetic construct
QQTAIYST

SEQ ID NO: 88 - protein, 9 aa, synthetic construct
QQYDSPLLT

SEQ ID NO: 89 - protein, 9 aa, synthetic construct
QQYHEYSPT

SEQ ID NO: 90 - protein, 9 aa, synthetic construct
QQYSMDHLT

SEQ ID NO: 91 - protein, 9 aa, synthetic construct
QQYNSFSIT

SEQ ID NO: 92 - protein, 9 aa, synthetic construct
QQSDTYPRT

SEQ ID NO: 93 - protein, 9 aa, synthetic construct
QQKDTWSYT

SEQ ID NO: 94 - protein, 8 aa, synthetic construct
QQYDQYWT

SEQ ID NO: 95 - protein, 10 aa, synthetic construct
QQTHSYSSTT

SEQ ID NO: 96 - protein, 8 aa, synthetic construct
QQVYIYST

SEQ ID NO: 97 - protein, 8 aa, synthetic construct
QQYGIPWT

SEQ ID NO: 98 - protein, 10 aa, synthetic construct
QQYTVYSPVT

SEQ ID NO: 99 - protein, 5 aa, synthetic construct
SYAIS

SEQ ID NO: 100 - protein, 5 aa, synthetic construct
SYAMS

SEQ ID NO: 101 - protein, 17 aa, synthetic construct
GIIPIFGTANYAQKFQG

SEQ ID NO: 102 - protein, 17 aa, synthetic construct
AISGSGGSTYYADSVKG

SEQ ID NO: 103 - protein, 10 aa, synthetic construct
GYFSEEAMDY

SEQ ID NO: 104 - protein, 10 aa, synthetic construct
AWYFGYDFDY

SEQ ID NO: 105 - protein, 11 aa, synthetic construct
GWYFFDAGFDY

SEQ ID NO: 106 - protein, 11 aa, synthetic construct
EWYWPDNPFDY

SEQ ID NO: 107 - protein, 12 aa, synthetic construct
APYSPTYYPFDY

SEQ ID NO: 108 - protein, 12 aa, synthetic construct
SYNWYEWYWFDY

SEQ ID NO: 109 - protein, 10 aa, synthetic construct
DWAGIYYLDY

SEQ ID NO: 110 - protein, 10 aa, synthetic construct
DWGGIYYFDY

SEQ ID NO: 111 - protein, 11 aa, synthetic construct
PSLADWGDMDY

SEQ ID NO: 112 - protein, 12 aa, synthetic construct
DWVYFGVGSFDY

SEQ ID NO: 113 - protein, 11 aa, synthetic construct
DYWLDSDYFDY

SEQ ID NO: 114 - protein, 10 aa, synthetic construct
DYGTISAFDY

SEQ ID NO: 115 - protein, 8 aa, synthetic construct
EYFYAMDY

SEQ ID NO: 116 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYDSYYTFGQGTKVEIKR

SEQ ID NO: 117 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQTAIYSTFGQGTKVEIKR

SEQ ID NO: 118 - protein, 108 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYDSPLLTFGQGTKVEIKR

SEQ ID NO: 119 - protein, 108 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYHEYSPTFGQGTKVEIKR

SEQ ID NO: 120 - protein, 108 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYSMDHLTFGQGTKVEIKR

SEQ ID NO: 121 - protein, 108 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYNSFSITFGQGTKVEIKR

SEQ ID NO: 122 - protein, 108 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQSDTYPRTFGQGTKVEIKR

SEQ ID NO: 123 - protein, 108 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQKDTWSYTFGQGTKVEIKR

SEQ ID NO: 124 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYDQYWTFGQGTKVEIKR

SEQ ID NO: 125 - protein, 109 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQTHSYSSTTFGQGTKVEIKR

SEQ ID NO: 126 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQVYIYSTFGQGTKVEIKR

SEQ ID NO: 127 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYGIPWTFGQGTKVEIKR

SEQ ID NO: 128 - protein, 109 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYTVYSPVTFGQGTKVEIKR

SEQ ID NO: 129 - protein, 119 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGYFSEEAMDYWGQGTTVTVSS

SEQ ID NO: 130 - protein, 119 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAWYFGYDFDYWGQGTTVTVSS

SEQ ID NO: 131 - protein, 120 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGWYFFDAGFDYWGQGTTVTVSS

SEQ ID NO: 132 - protein, 120 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREWYWPDNPFDYWGQGTTVTVSS

SEQ ID NO: 133 - protein, 121 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPYSPTYYPFDYWGQGTTVTVSS

SEQ ID NO: 134 - protein, 121 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARSYNWYEWYWFDYWGQGTLVTVSS

SEQ ID NO: 135 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWAGIYYLDYWGQGTLVTVSS

SEQ ID NO: 136 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWGGIYYFDYWGQGTLVTVSS

SEQ ID NO: 137 - protein, 120 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARPSLADWGDMDYWGQGTLVTVSS

SEQ ID NO: 138 - protein, 121 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDWVYFGVGSFDYWGQGTLVTVSS

SEQ ID NO: 139 - protein, 120 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDYWLDSDYFDYWGQGTLVTVSS

SEQ ID NO: 140 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDYGTISAFDYWGQGTLVTVSS

SEQ ID NO: 141 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEYFYAMDYWGQGTLVTVSS

SEQ ID NO: 142 - DNA, 20 bp, synthetic construct
  source 1..20: mol_type = other DNA
agagtctctcagctggtaca

SEQ ID NO: 143 - DNA, 20 bp, synthetic construct
  source 1..20: mol_type = other DNA
gagtagcgcgagcacagcta

SEQ ID NO: 144 - DNA, 20 bp, synthetic construct
  source 1..20: mol_type = other DNA
gcacttggtattctgggtcc